(19) United States
(12) Patent Application Publication
(10) Pub. No.: US 2021/0191198 A1
(43) Pub. Date: Jun. 24, 2021

(54) ASSISTANT DYES FOR COLOR CONVERSION IN LCD DISPLAYS

(71) Applicant: MolecuLED Ltd., Herzeliya (IL)

(72) Inventors: Sanaa MUSA, Deir EI Assad (IL); Rony SCHWARZ, Kibbutz Ma'anit (IL); Eran SELLA, Tel-Aviv (IL); Daniel SZWARCMAN, Pardes-Hanna Karkur (IL); Maxim LIBERMAN, Haifa (IL); Evgenia Liel (Jeny) KUKS, Ramat Gan (IL); Ziv SOBOL, Ra'anana (IL); Daniel ARONOV, Netanya (IL); Mor Shmuel ARMON, Ramat-Gan (IL); Elad Cohen, Tel Aviv (IL)

(73) Assignee: MolecuLED Ltd., Herzeliya (IL)

(21) Appl. No.: 16/718,245

(22) Filed: Dec. 18, 2019

(51) Int. Cl. G02F 1/1335

(52) U.S. Cl. CPC G02F 1/133609; G02F 1/133514; G02F 1/133606; G02F 1/133603

(57) ABSTRACT

LCDs (liquid crystal displays) with improved efficiency and performance, as well as corresponding methods are disclosed. Color conversion films and elements with rhodamine-based fluorescent compounds and/or assistant dyes are used to modify the spectrum of the illumination provided by the backlight unit in either or both the backlight unit itself and the LCD panel, in various configurations. Color conversion may be performed above the LC module, possibly by a patterned layer incorporating the color filters, and/or within the backlight unit within a fluorescence-intensifying section in which radiation is recycled to enhance color conversion. Film configuration, positions and optionally supportive structures are provided, to extend the lifetime of the fluorescent compounds. Collimation of backlight illumination may further enhance the optical performance of disclosed LCDs.

### BACKGROUND OF THE INVENTION

### 1. Technical Field

The present invention relates to the field of color conversion films in displays, and more particularly, to color conversion films with fluorescent compounds.

### 2. Discussion of Related Art

Improving displays with respect to their energy efficiency and color gamut performance is an ongoing challenge in the industry. While color conversion films are available which use quantum dots to enhance display performance, it is particularly challenging to achieve comparable goals in ways that do not involve heavy metals such as toxic cadmium used in quantum dots.

Constant developments in the field of liquid crystal displays concern improvements of optical and visual performance, increasing the displayed intensity while complying to white point requirements.

LCDs are continuously being developed, with performance improvements taking place in different components of the displays, such as the backlight units, liquid crystal module, layering of the LCD panel and optimization of optical performance of the displays.

### SUMMARY OF THE INVENTION

The following is a simplified summary providing an initial understanding of the invention. The summary does not necessarily identify key elements nor limit the scope of the invention, but merely serves as an introduction to the following description.

One aspect of the present invention provides A LCD (liquid crystal display) comprising: a backlight unit, and a LCD panel receiving illumination radiation from the backlight unit, the LCD panel comprising: a liquid crystal (LC) module, RGB (red, green, blue) color filters (common ranges are Red: 635-700 nm, Green: 520-560 nm and Blue: 450-490 nm, the exact ranges can be tuned according to desired specifications), and at least one color conversion film comprising: at least one red-fluorescent rhodamine-based fluorescent (RBF) compound selected to have at least one R (red) emission peak, at least one green RBF compound selected to have at least one G (green) emission peak, and at least one assistant dye configured to modify a spectrum of received radiation.

These, additional, and/or other aspects and/or advantages of the present invention are set forth in the detailed description which follows; possibly inferable from the detailed description; and/or learnable by practice of the present invention.

### BRIEF DESCRIPTION OF THE DRAWINGS

For a better understanding of embodiments of the invention and to show how the same may be carried into effect, reference will now be made, purely by way of example, to the accompanying drawings in which like numerals designate corresponding elements or sections throughout.

In the accompanying drawings:

FIG. 1A is a high level schematic overview illustration of disclosed configurations of displays, according to some embodiments of the invention.

FIG. 1B is a high level schematic overview illustration of disclosed film production processes, film configurations and display configurations, according to some embodiments of the invention.

FIG. 1C is a high-level schematic exploded view of a LCD having a collimated backlight unit, according to some embodiments of the invention.

FIG. 1D is a high level schematic block diagram illustrating various configurations of LCD panels and displays, according to some embodiments of the invention.

FIGS. 2A-2L are high level schematic illustrations of configurations of digital displays with color conversion film(s), according to some embodiments of the invention.

FIG. 2M is a high-level schematic illustration of patterned color conversion film(s) with matrix-like crosstalk-reducing layer(s) in an above-LC configuration, according to some embodiments of the invention.

FIGS. 2N and 2S are high-level schematic illustrations of the LCD panel comprising the color conversion and filtering layer above the LC module, with a top optical-elements array, according to some embodiments of the invention.

FIGS. 2O-2R are high-level schematic illustrations of a part of the LCD panel, according to some embodiments of the invention.

FIG. 3A is a high-level schematic layered view of a LCD having a collimated backlight unit, according to some embodiments of the invention.

FIGS. 3B and 3C are high-level schematic illustrations of illumination units with designed serrated lens and an additional lens providing collimated illumination, according to some embodiments of the invention.

FIG. 4 is a high-level schematic illustration of a light source layer and illumination units with array of optical elements, configured to provide collimated backlight illumination, according to some embodiments of the invention.

FIGS. 5A-5J are high-level schematic illustrations of illumination units and light source layers, according to some embodiments of the invention.

FIG. 6A is a high-level schematic illustration of a light source layer with lateral illumination source(s) and common internally reflective cavity(ies) having multiple pinpoint openings, configured to provide collimated backlight illumination, according to some embodiments of the invention.

FIG. 6B is a high-level schematic illustration of a light source layer with multiple pinpoint openings and associated optical elements for each illumination source and internally reflective cavity, configured to provide collimated backlight illumination, according to some embodiments of the invention.

FIGS. 7A-7F are high-level schematic illustrations of illumination units, according to some embodiments of the invention.

FIGS. 8A-8E are high level schematic illustrations of configurations of digital displays with color conversion film(s), according to some embodiments of the invention.

FIG. 9 is an illustration example of polarization anisotropy of film(s) with RBF (rhodamine-based fluorescent) compound(s), according to some embodiments of the invention.

FIGS. 10A and 10B are high level schematic illustration of spectral enhancements in devices with white illumination, according to some embodiments of the invention.

FIGS. 10C-10E are high level schematic illustrations of spectrum shaping using assistant dyes, according to some embodiments of the invention.

FIG. 11 is a high level schematic illustration of multiple film preparation steps and processes, according to some embodiments of the invention.

FIG. 12 is a high-level schematic illustration of a backlight unit (BLU) for a liquid crystal display (LCD), according to some embodiments of the invention.

FIG. 13 is a high-level schematic illustration of white point adjustment for LCD, according to some embodiments of the invention.

FIGS. 14A-F are high-level schematic illustrations of BLUs with color conversion unit having partly reflective structure(s), according to some embodiments of the invention.

FIGS. 15A-15E and 16A-16D are high-level schematic illustrations of BLUs having color conversion unit in which the color conversion elements receive only part of the overall radiation, according to some embodiments of the invention.

FIG. 17 is a high-level flowchart illustrating methods, according to some embodiments of the invention.

### DETAILED DESCRIPTION OF THE INVENTION

In the following description, various aspects of the present invention are described. For purposes of explanation, specific configurations and details are set forth in order to provide a thorough understanding of the present invention. However, it will also be apparent to one skilled in the art that the present invention may be practiced without the specific details presented herein. Furthermore, well known features may have been omitted or simplified in order not to obscure the present invention. With specific reference to the drawings, it is stressed that the particulars shown are by way of example and for purposes of illustrative discussion of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments that may be practiced or carried out in various ways as well as to combinations of the disclosed embodiments. Also, it is to be understood that the phraseology and terminology employed herein is for the purpose of description and should not be regarded as limiting.

LCDs (liquid crystal displays) with improved efficiency and performance, as well as corresponding methods are disclosed. Color conversion films and elements with rhodamine-based fluorescent compounds and/or assistant dyes are used to modify the spectrum of the illumination provided by the backlight unit in either or both the backlight unit itself and the LCD panel, in various configurations. Color conversion may be performed above the LC (liquid crystal) module, possibly by a patterned layer incorporating the color filters, and/or within the backlight unit within a fluorescence-intensifying section in which radiation is recycled to enhance color conversion. Film configuration, positions and optionally supportive structures are provided, to extend the lifetime of the fluorescent compounds. Collimation of backlight illumination may further enhance the optical performance of disclosed LCDs.

Facing the challenge of improving the efficiency and color performance of displays without having to rely on compounds involved in displays containing quantum-dot-based technologies (e.g., in color filters, color conversion materials etc.), the inventors have discovered ways of using organic molecules to significantly improve display properties. In the following, display configurations are presented with respect to the use of color conversion films and then sol-gel and UV (ultraviolet) technologies are disclosed for preparing color conversion films as well as for preparing associated protective films or coatings for the color conversion films.

Embodiments of the present invention provide efficient and economical methods and mechanisms for constructing and operating LCDs. Backlight units, LCDs and methods are provided, which utilize collimated illumination which improves the performance of the LCDs. Backlight units comprise illumination source(s) configured to provide illumination, reflective cavity(ies) configured to receive the provided illumination, pinpoint openings in the internally reflective cavity(ies), which are arranged in a plane and have opening areas of below a specified size, and an array of optical elements configured to collimate illumination exiting from the pinpoint openings. The collimated illumination improves the spatial accuracy of the LCD and enables efficiency improvements using color conversion films, e.g., in above-panel configurations, with separate or integrated color conversion film and color filters layer consecutive to the LC module, without scattering losses and parallax inaccuracies. Certain embodiments may reduce parallax issues in multilayered LC panels, and enable implementation of local dimming for supporting high dynamic range (HDR) displays.

Embodiments of the present invention provide efficient and economical methods and mechanisms for improving LCD backlight units and LCD white points using color conversion elements. Backlight units (BLUs) for liquid crystal displays (LCDs) are provided, as well as methods of enhancing color conversion in BLUs. BLUs comprise color conversion unit(s) having partly reflective structure(s) and color conversion element(s), and illumination source(s) configured to deliver radiation through the color conversion unit(s) to the LCD panel of the LCD. Color conversion element(s) may comprise fluorescent dye(s) configured to convert blue radiation to green and/or red radiation. The partly reflective structure(s) are configured to redirect at least a part of the delivered radiation to pass multiple times through the color conversion element(s) to thereby (and by color conversion elements configuration) set a white point of the delivered radiation according to specified requirements. Some of the blue radiation may be delivered directly to the LCD panel, to enhance the lifetime of the LCD and/or of the fluorescent dyes.

Color conversion films for a LCD (liquid crystal display) having RGB (red, green, blue) color filters (having red, green and blue filtering ranges, configured to comply with any of various standards), as well as such displays, formulations, precursors and methods are provided, which improve display performances with respect to color gamut, energy efficiency, materials and costs. The color conversion films absorb backlight illumination and convert the energy to green and/or red emission at high efficiency, specified wavelength ranges and narrow emission peaks. For example, rhodamine-based fluorescent compounds are used in matrices produced by sol gel processes and/or UV (ultraviolet) curing processes which are configured to stabilize the compounds and extend their lifetime—to provide the required emission specifications of the color conversion films. Film integration and display configurations further enhance the display performance with color conversion films utilizing various color conversion elements and possibly patterned and/or integrated with a crosstalk blocking matrix. For example, the color conversion film(s) may be integrated in the LCD panel below the color filters, either before or after the analyzer associated with the liquid crystal film.

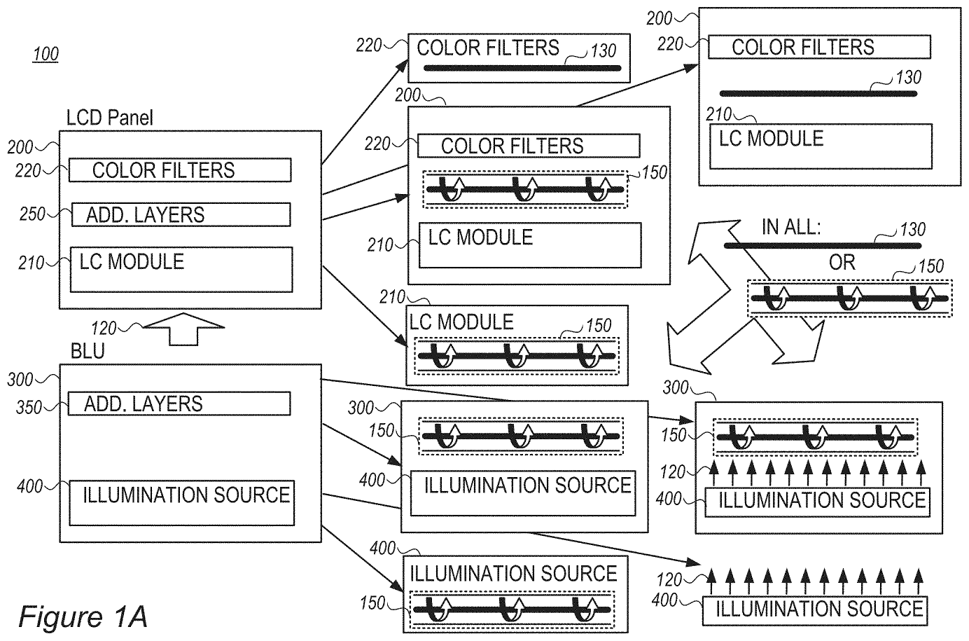

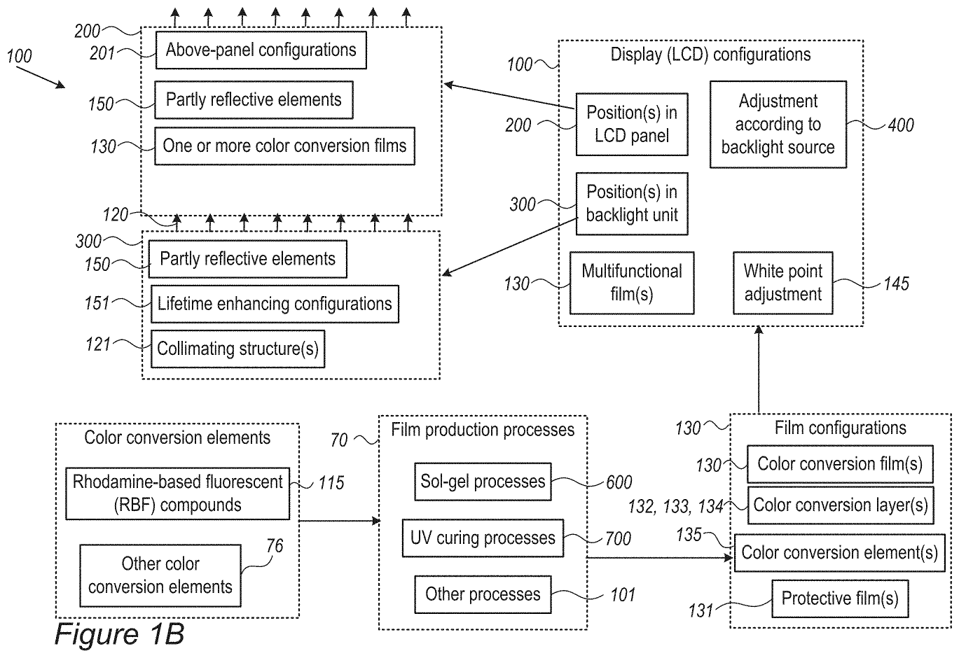

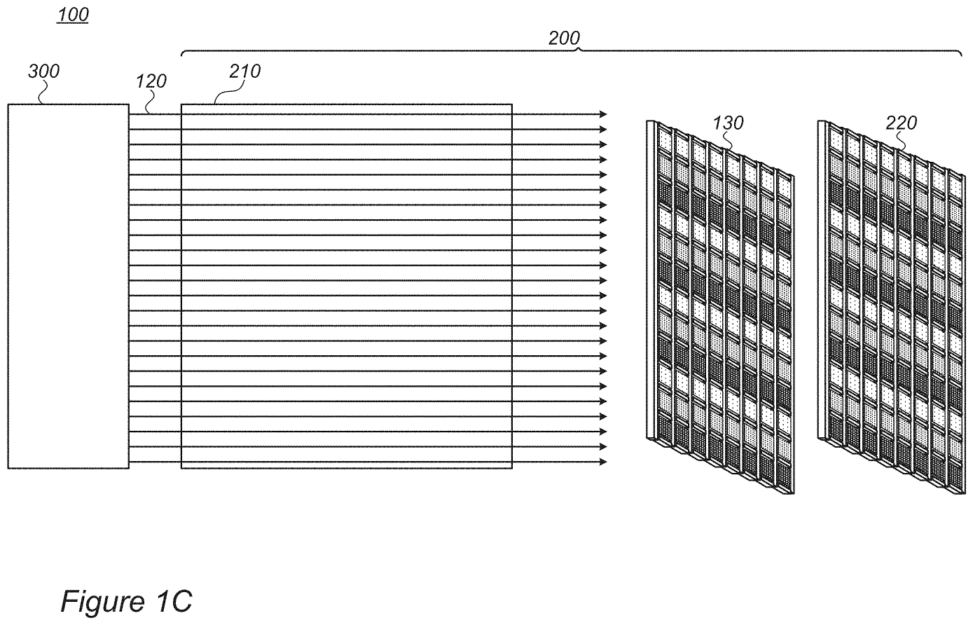

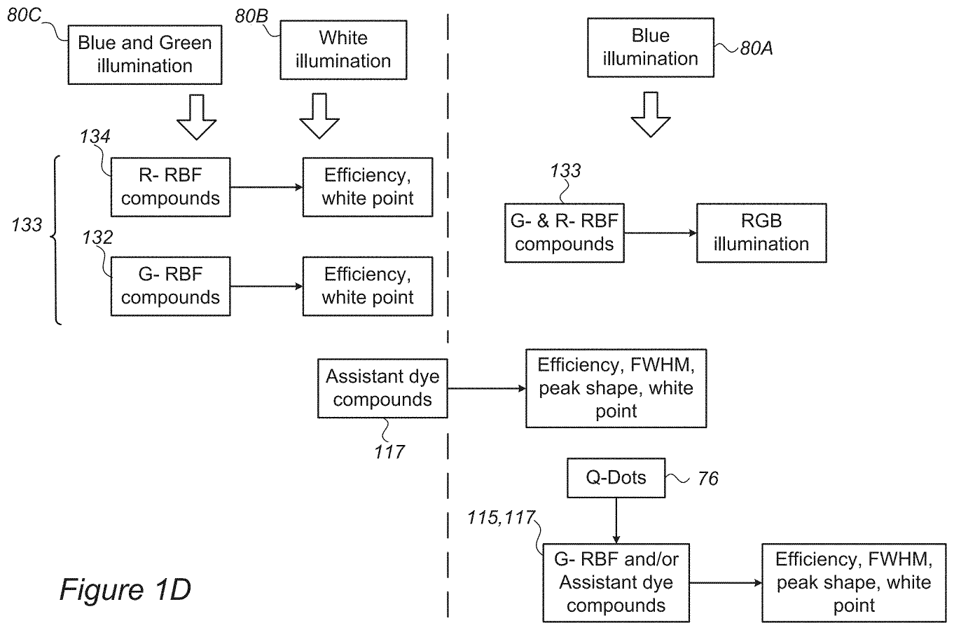

FIG. 1A is a high level schematic overview illustration of disclosed configurations of display 100, according to some embodiments of the invention. Schematically, display 100 comprises a backlight unit 300 (BLU) comprising an illumination source 400 and additional layers 350 (e.g., diffuser(s), prism layer(s) etc., see below), and a LCD panel 200 comprising a LC (liquid crystal) module 210 modifying illumination 120 received from BLU 300 and color filters 220 (e.g., RGB—red, green and blue filtering elements), both LC module 210 and color filters 220 are typically patterned into pixels and subpixels, and additional layers 250 (see examples below).

It is noted that while the numerals 300 and 400 are used herein to denote the BLU and the modified illumination sources, respectively, certain embodiments comprise extended illumination sources 400 which may be used as BLUs 300. In such cases, either numeral, 300 or 400, is applicable. In other cases, with BLU 300 comprising additional layers 350, the numerals are clearly distinct. It is further noted that various embodiments may comprise combinations of illumination source 400 and layers 350, which are disclosed in different embodiments.

One or more color conversion layers 130 are introduced herein, which are configured to improve any of a number of display performance parameters such as display energy use efficiency, display lifetime, display color performance (e.g., color gamut) etc. Color conversion layer(s) 130 may be implemented in LCD panel 200 and/or in BLU 300, possibly in combination with supportive structures, e.g., as a fluorescence-intensifying section 150 which enhances fluorescence output and possibly extends the lifetime of fluorescent molecules in cases color conversion layers 130 are based on fluorescent molecules. In any of the disclosed display configuration, either or both color conversion layer 130 and fluorescence-intensifying section 150 may be used interchangeably, or in combination, according to specified performance requirements. Various embodiments disclosed below may be combined into specified display configurations upon requirement.

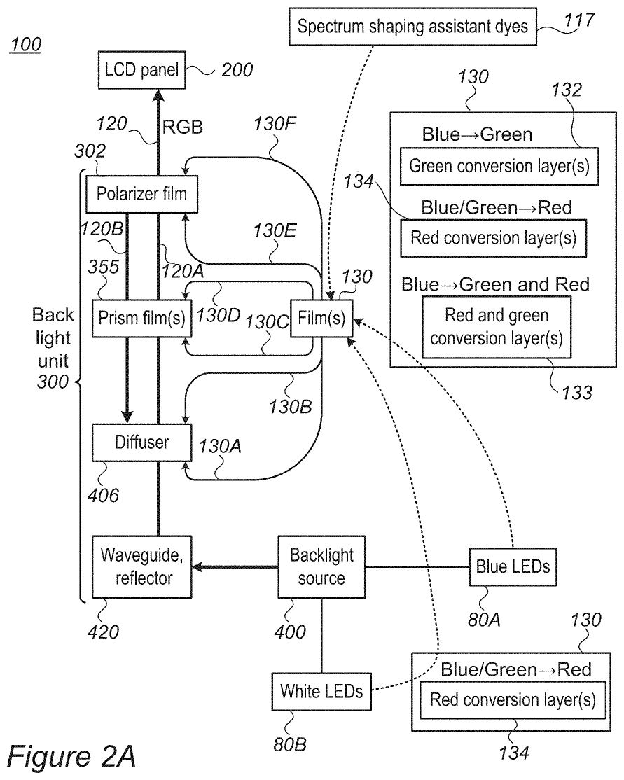

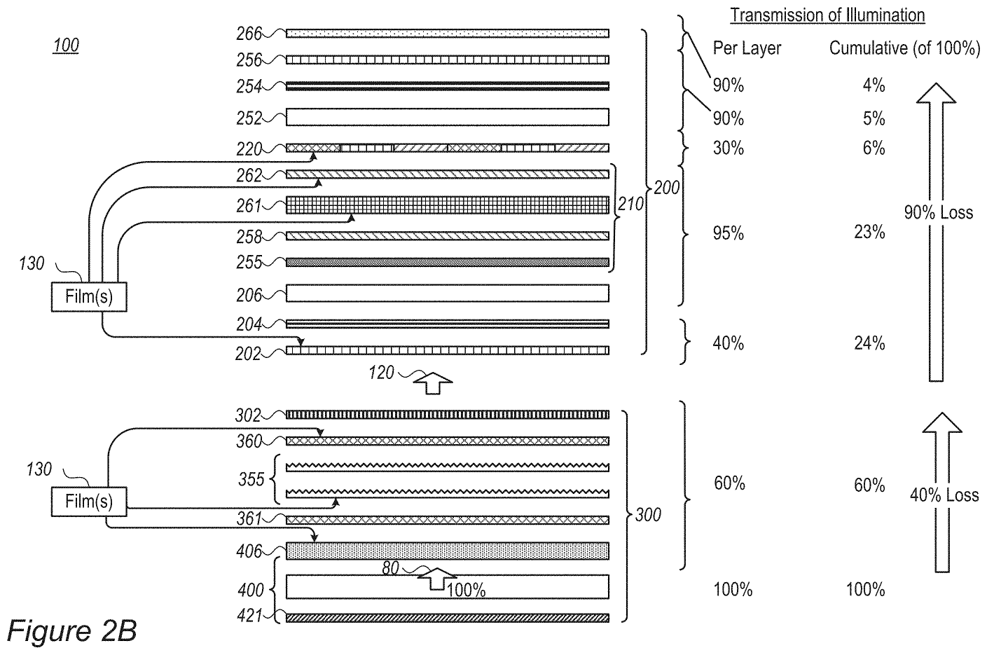

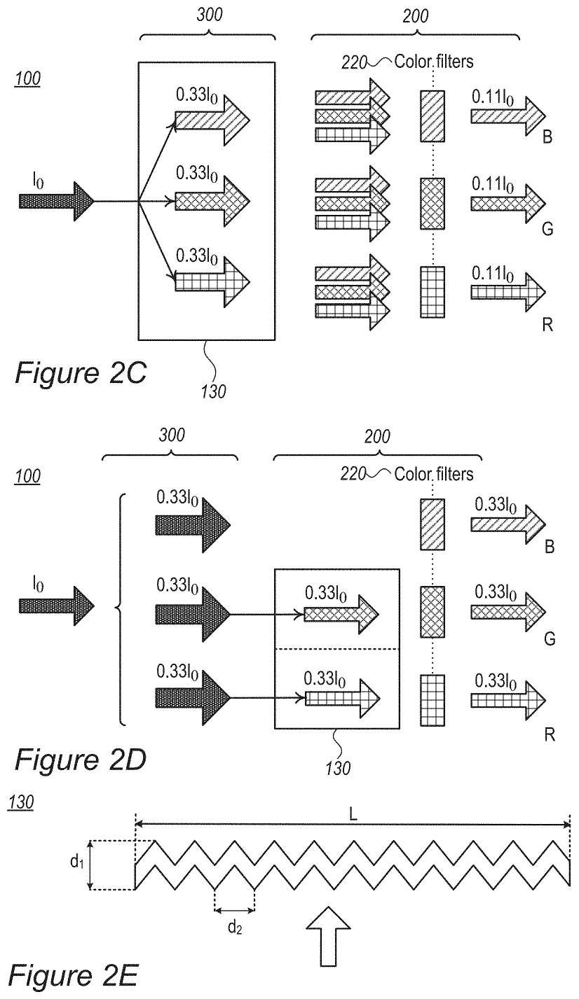

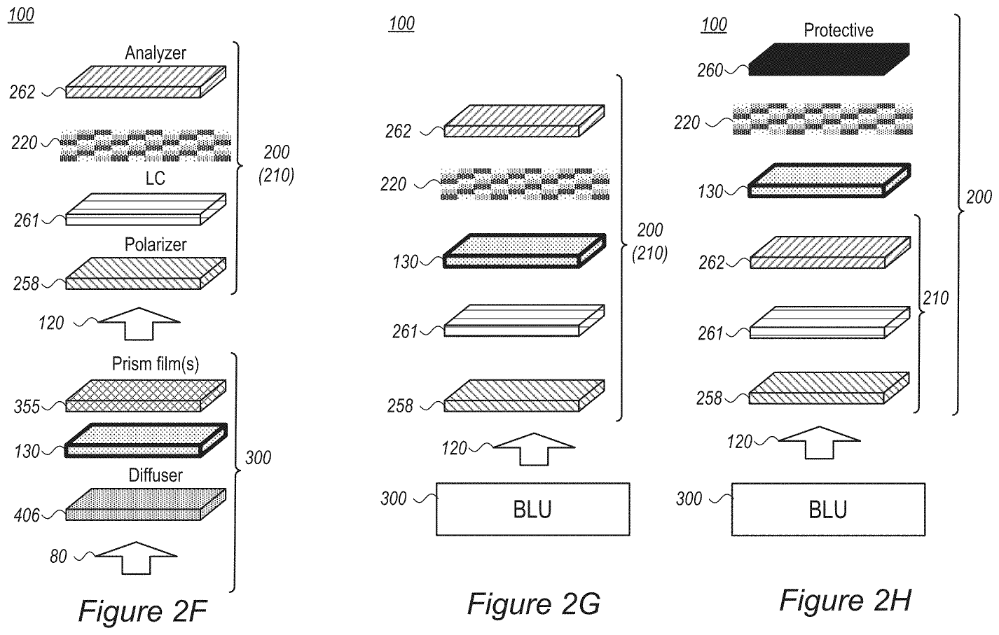

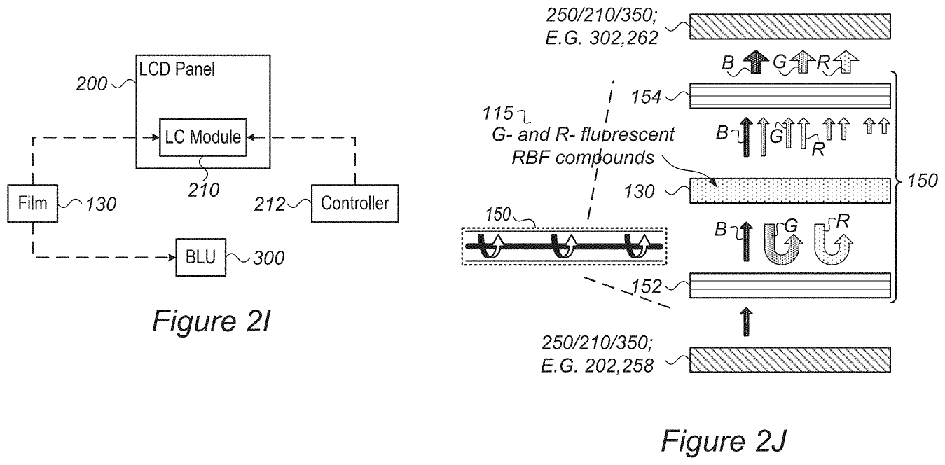

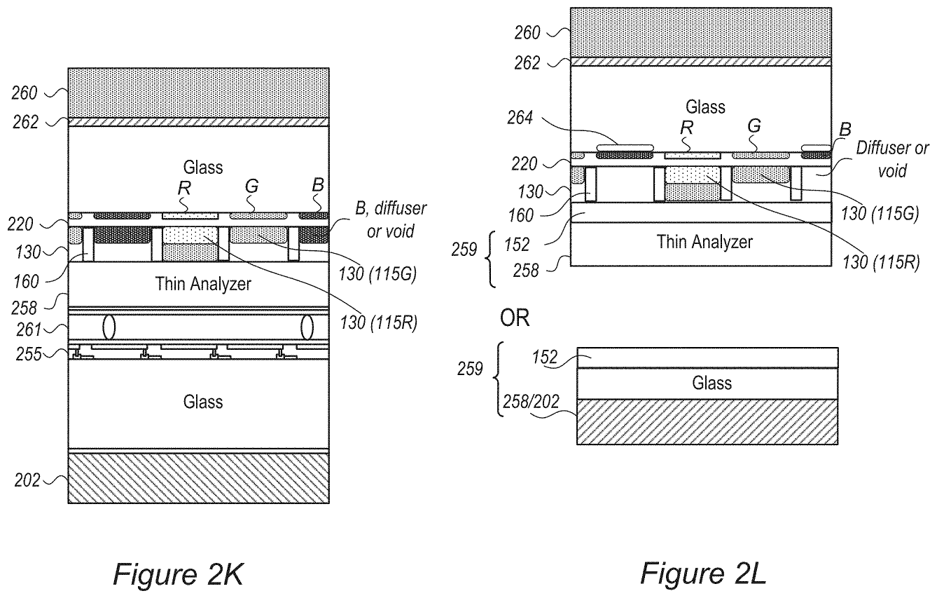

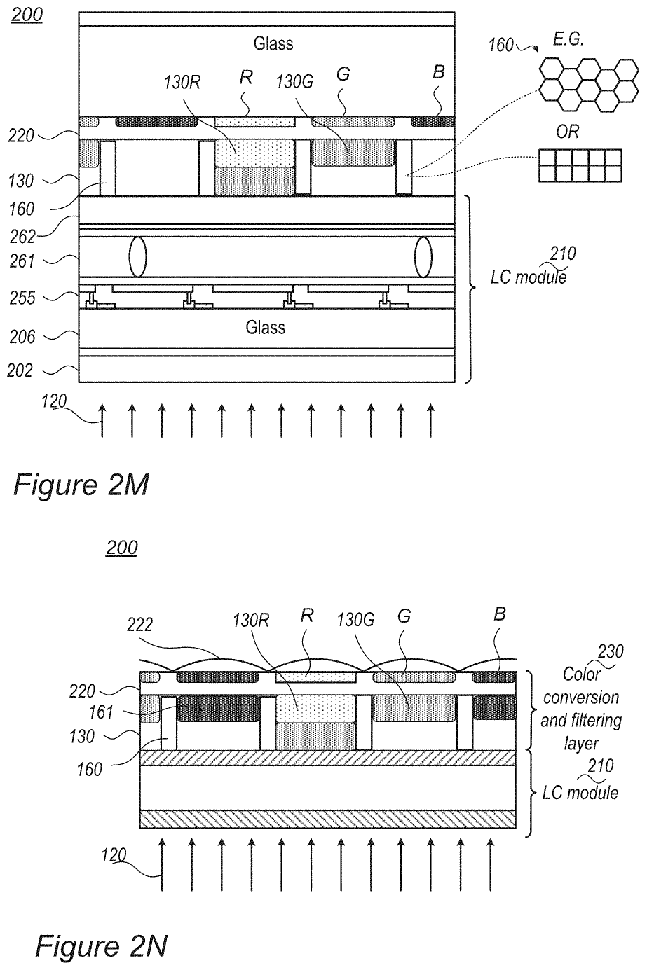

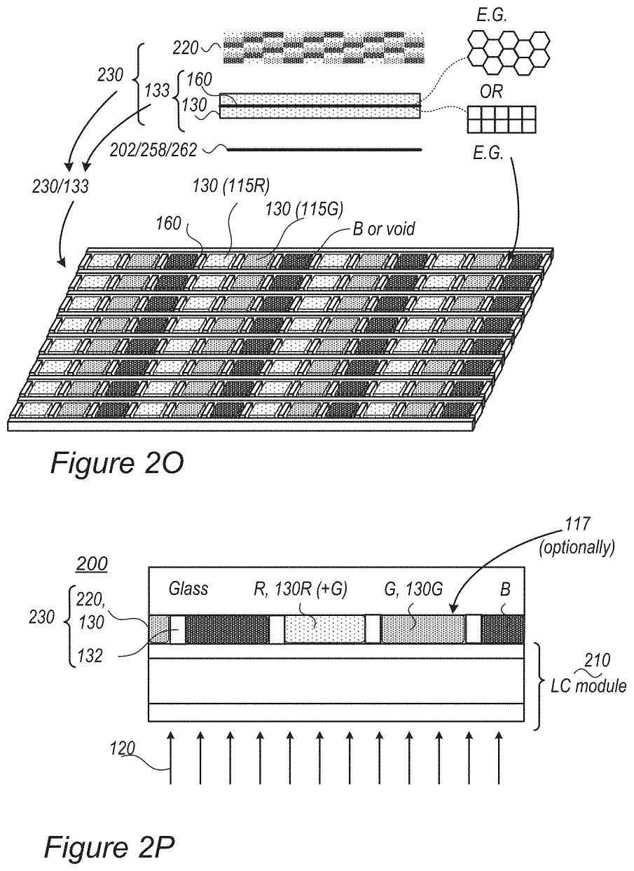

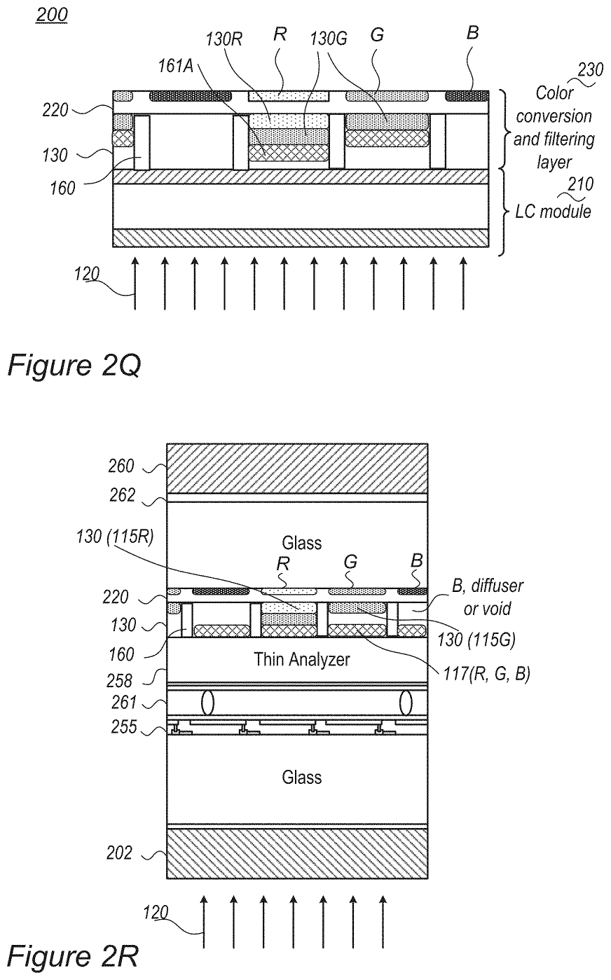

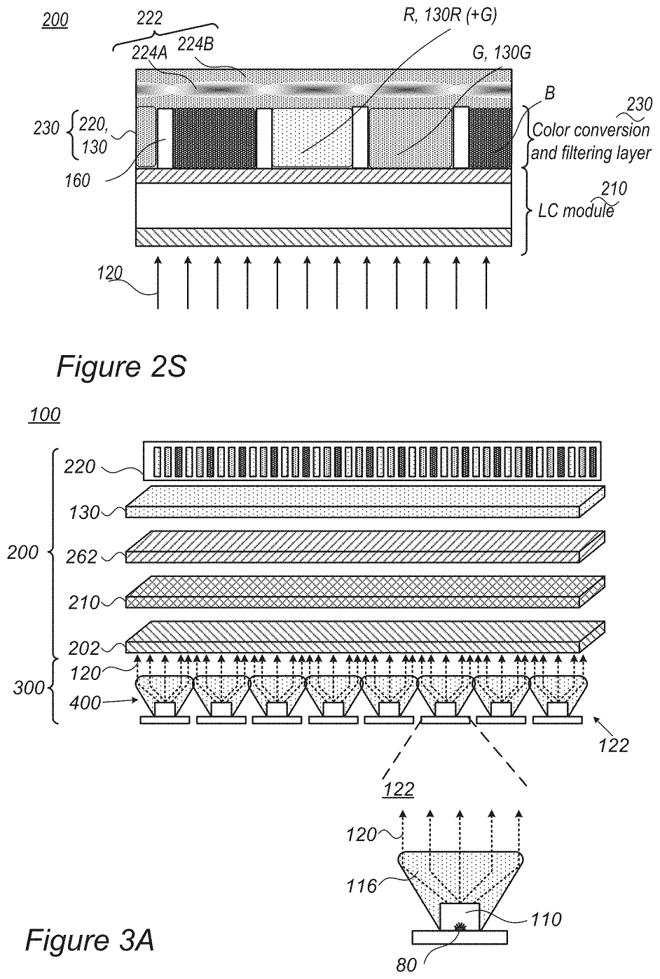

In certain embodiments, color conversion layer(s) 130 may be used in adjacency or in combination with color filters 220, either as adjacent layers or an integrated layer, possibly further comprising patterning elements (see, e.g., FIGS. 2O, 2P, 2R, 2S). Color filters 220 with color conversion layer 130 may be positioned above LC module 210 (see, e.g., FIGS. 2H and 2K-2N) or within LC module 210 (see, e.g., FIG. 2G).

In certain embodiments, fluorescence-intensifying section 150 (and/or color conversion layer(s) 130) may be positioned between LC module 210 and color filters 220, possibly with additional patterning elements (see, e.g., FIGS. 2K-2N) and possibly upon enhanced collimation of illumination 120 from BLU 300.

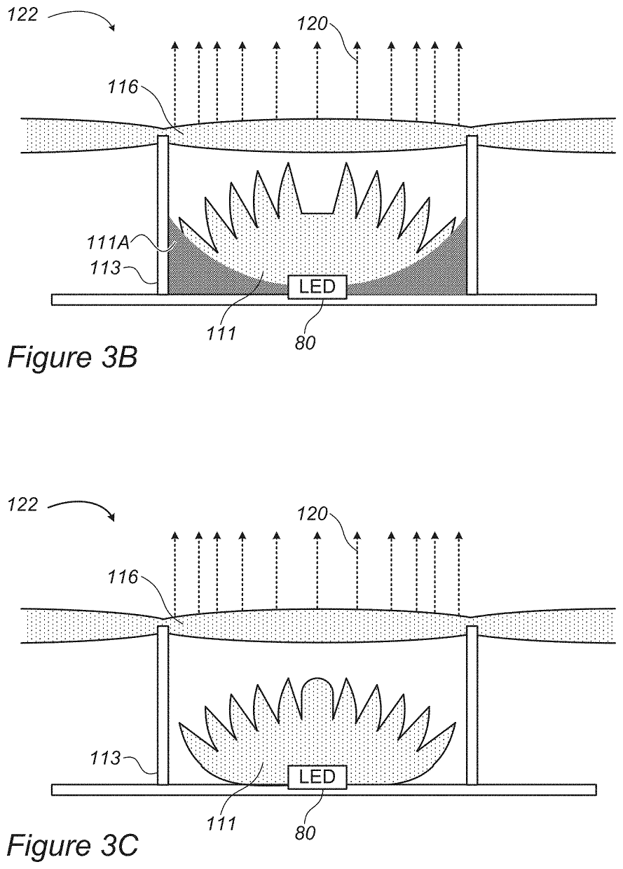

In certain embodiments, fluorescence-intensifying section 150 (and/or color conversion layer(s) 130) may be positioned within LC module 210, and configured to maintain the operability of LC module 210, possibly using additional patterning elements and/or by modifying BLU 300 to enhance the degree of collimation of illumination 120 provided thereby (see, e.g., FIGS. 3A-3C).

In certain embodiments, fluorescence-intensifying section 150 (and/or color conversion layer(s) 130) may be positioned within BLU 300, either or both above illumination source 400 or within illumination source 400, as illustrated e.g., in FIGS. 2F and 14A-16B.

In certain embodiments, illumination source 400 may be modified to deliver collimated illumination 120 (see e.g. FIGS. 3A-7F), to improve the performance of LCD panel 200, possibly compensating for some straying of light which may be caused by the addition of fluorescence-intensifying section 150 and/or color conversion layer(s) 130 in BLU 300 and/or in LCD panel 200.

FIG. 1B is a high level schematic overview illustration of disclosed film production processes 70, film configurations 130 and display configurations 100, according to some embodiments of the invention. Embodiments combine color conversion elements (such as rhodamine-based fluorescent (RBF) compounds 115 and/or other color conversion elements 76 such as fluorescent organic and/or inorganic compounds, quantum dots etc.) into films 130 by various film production processes 70 (such as sol gel processes 600, UV curing processes 700 and/or other processes 101) to yield a variety of film configurations 130 such as color conversion films 130 and/or protective films 131 (which may be also color conversion films 130), which are then used in a variety of display configurations 100. Any of films 130 and 131 and layers 132, 133, 134 and possibly color conversion element(s) 135 discussed below may be prepared by sol gel processes 600 and/or UV curing processes 700. Film(s) 130 and 131 and layers 132, 133, 134 and possibly color conversion element(s) 135 may be used in display(s) 100 in one or more ways, such as any of: positioned in one or more locations in a backlight unit 300 and/or in LCD panel 200 and used as multifunctional films 130 (e.g., configured to function as any of: color conversions films, protective films, diffusers, polarizers etc.). Further display configurations 100 may comprise adjusting film(s) 130 according to the backlight source 400 (see e.g., red enhancement below, possibly also green enhancement) and/or adjusting the display white point 145, adjustment which may be carried out by modifying any of the color conversion elements, film production processes 70 and/or film configurations 130. Some embodiments provide integrative approaches to display configuration, which take into account multiple factors at all illustrated levels, as exemplified below.

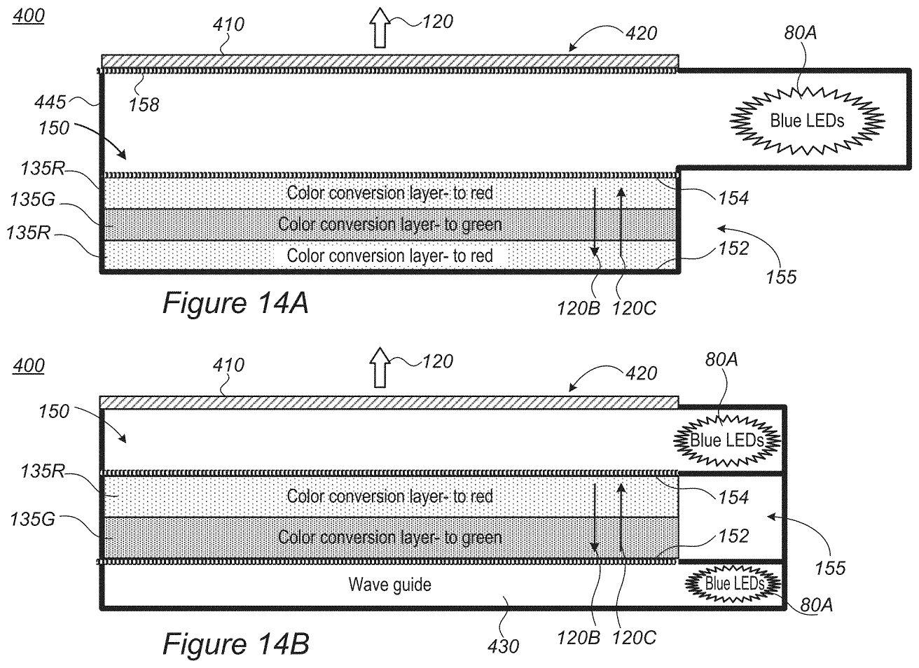

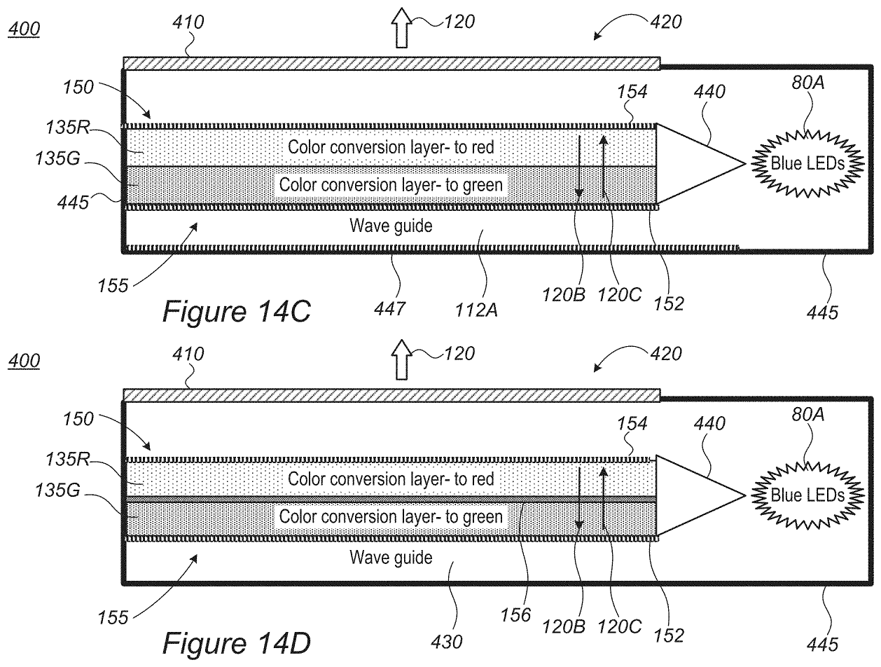

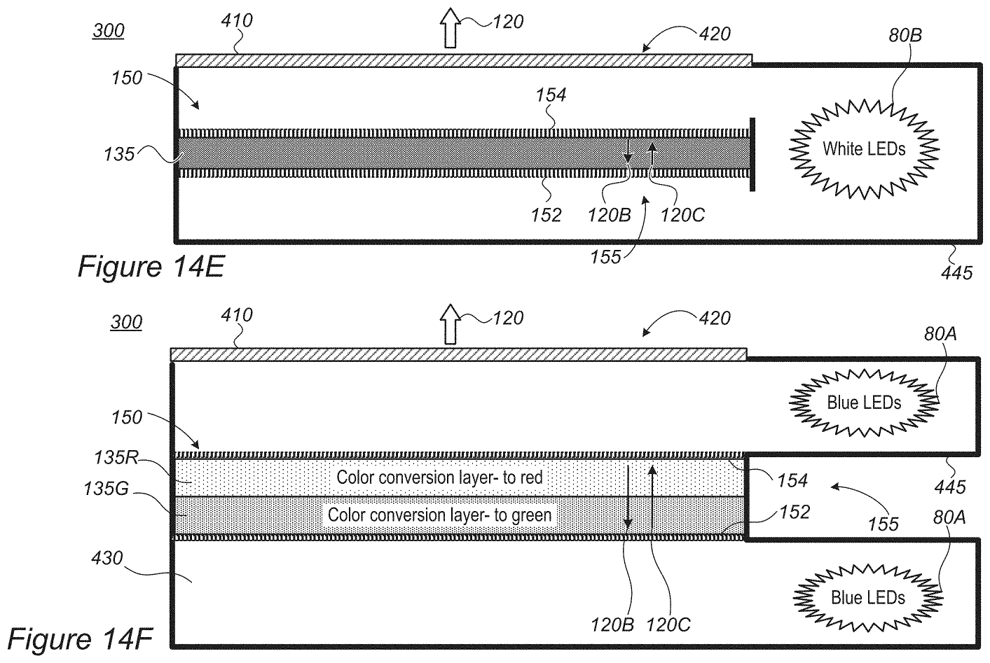

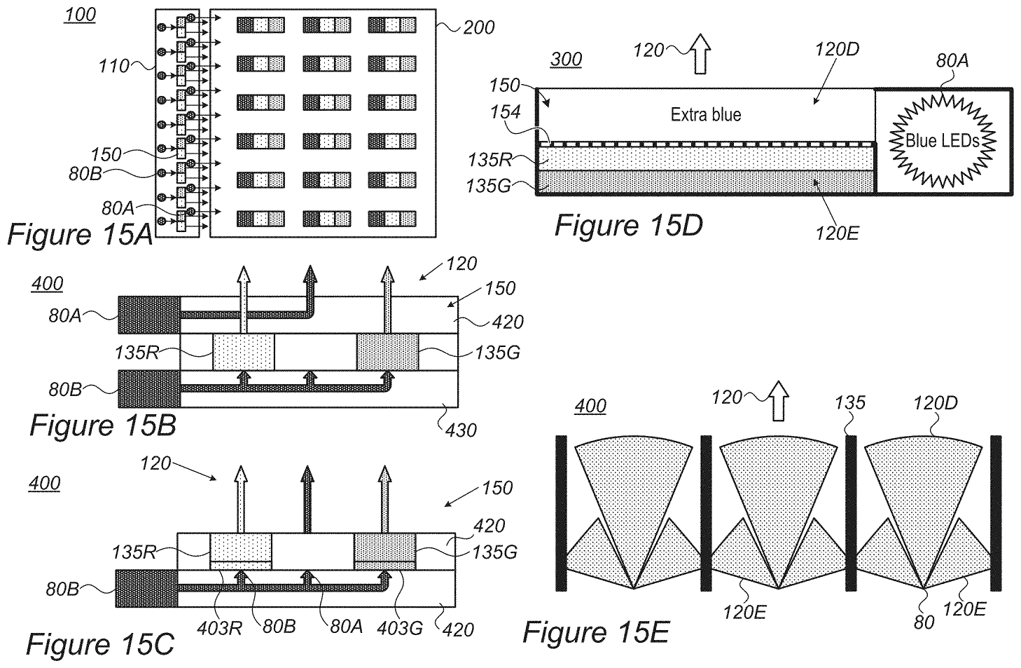

Embodiments of display configurations 100 comprise various combinations of elements of the present disclosure, such as above-panel configurations 201 comprising positions of color conversion layer 130 above LC module 210 (see, e.g., FIGS. 2H and 2K-2N), partly reflective elements implementing fluorescence-intensifying section 150, and one or more embodiments of color conversion films 130; as well as various BLU modifications such as partly reflective elements implementing fluorescence-intensifying section 150 in BLU 300 (see, e.g., FIGS. 14A-F), dye-lifetime enhancing configurations 151 (see, e.g., FIGS. 15A-E) and/or collimating structures 121 (see e.g. FIGS. 3A-7F), as illustrated below.

FIG. 1C is a high-level schematic exploded view of a LCD 100 having a collimated backlight unit 300, according to some embodiments of the invention. FIG. 1C provides a schematic example for above-panel configurations 201, possibly implementing collimating structures 121 in BLU 300. In certain embodiments, light source 300 may provide blue illumination 120 (from blue illumination source 80A) which is collimated, composed of parallel beams. LCD panel 200 may comprise LC module 210 having liquid crystal (LC) layer with associated polarizers and control circuitry (not shown), which is configured to control the images of LCD 100, with a color conversion film 130 and color filter layer 220 (which may be separate or integrated) following, to provide the displayed image. The above-display configuration of color conversion film 130 and color filter layer 220 is enabled by the fact that illumination 120 is collimated, preventing spatial discrepancies (such as scattering and cross talk) between positions of LC elements and positions of color filter elements.

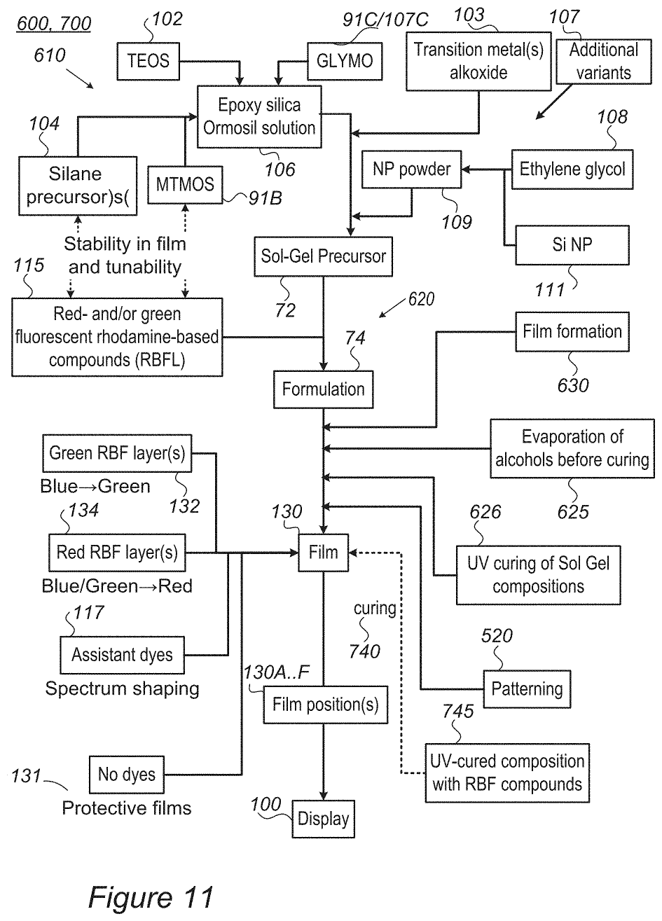

It is noted that any of the disclosed embodiments may be implemented in various pixel arrangements (e.g., stripe, mosaic, delta and boomerang arrangements, as non-limiting examples) and with respect to any number of subpixel per pixel (e.g., 1, 2, 3 or more subpixels per pixel, possibly with various color allocations per subpixel), possibly with corresponding spatial adjustments and configurations, and possibly only to some of the sub-pixels in the array. Clearly, the patterning of color conversion film 130 (see e.g., schematic illustration of patterning 520 in FIG. 11) may be configured to follow the patterning of color filter layer 220 and/or be integrated therewith. Elements of color conversion film 130 may be configured to be produced together with color filter layer 220 with minimal or possibly no additional complexity, using same or possibly modified production processes.

FIG. 1D is a high level schematic block diagram illustrating various configurations of LCD panel 200 and display 100, according to some embodiments of the invention. Various configurations and combinations illustrated in FIG. 1D are explained in more detail and demonstrated below. Disclosed configurations may be implemented for backlight units 300 configured to provide white illumination 80B (e.g., using white LEDs) and/or blue illumination 80A (e.g., using blue LEDs), as discussed below.

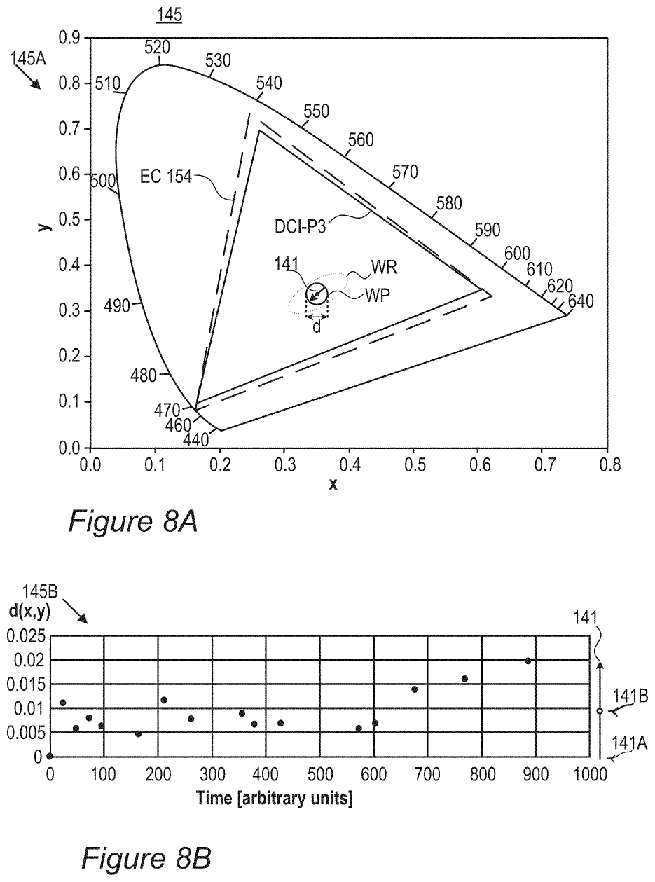

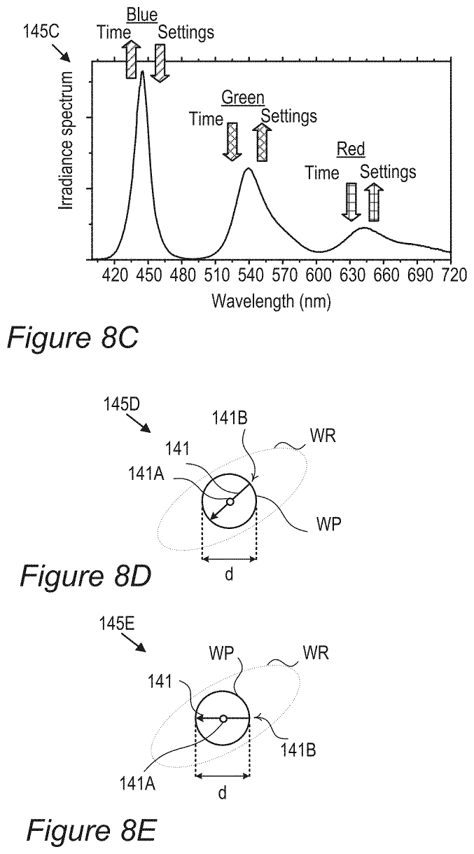

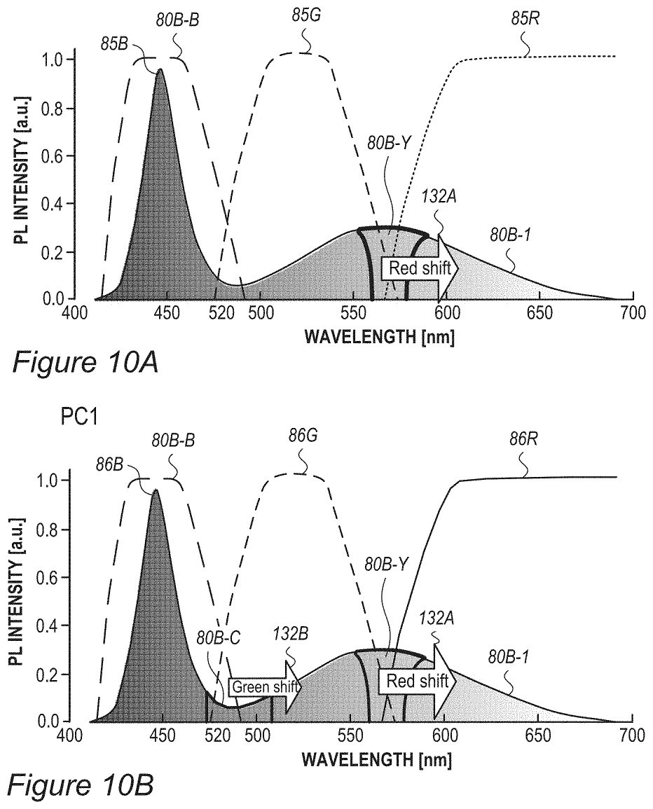

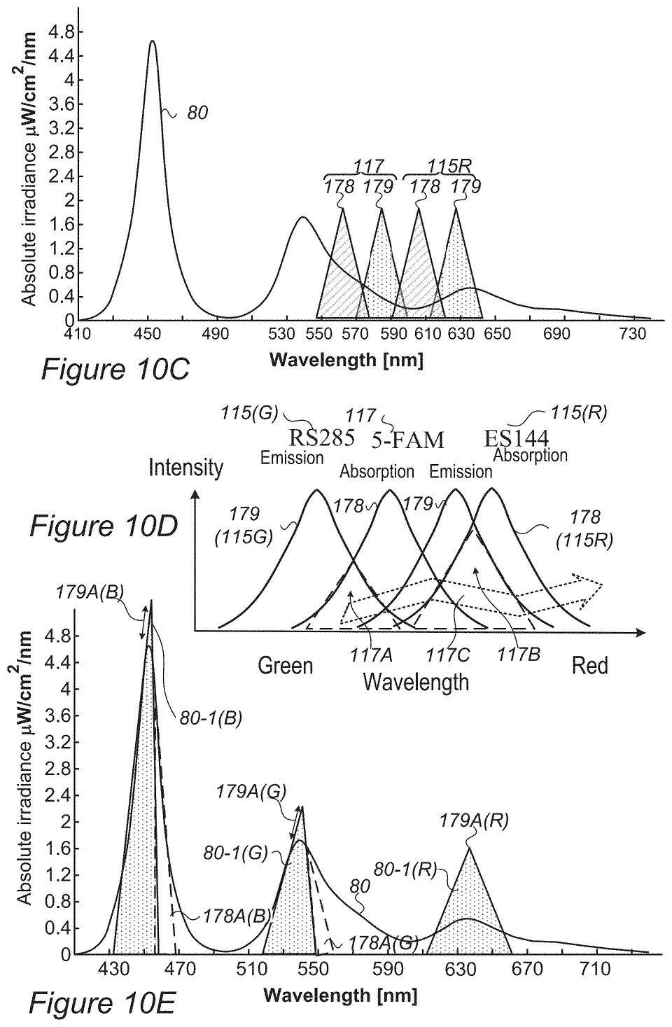

For white illumination 80B, red-fluorescent and green-fluorescent RBF compounds 115 in respective layers 134, 132 (or possibly in mixed layer 133) may be used to enhance efficiency (illumination intensity of LCD display 100) and/or adjust its white point. Efficiency enhancement may be achieved by changing the white illumination spectrum to bring a larger part of the spectrum into the transmission ranges of RGB filters 220, as illustrated e.g., in FIGS. 10A-10E and the respective disclosure sections. White point adjustment may be achieved by changing the ratios between the illumination components in the transmission ranges of RGB filters 220 within the illumination spectrum, as illustrated e.g., in FIGS. 8A-8E and the respective disclosure sections.

For blue illumination 80A, red-fluorescent and green-fluorescent RBF compounds 115 in one or more layers 133 may be used to adapt the illumination spectrum to the transmission ranges of RGB filters 220, as disclosed herein (see also FIG. 2D).

It is noted that the configuration of red-fluorescent and green-fluorescent RBF compounds 115 in color conversion films 130 or color conversion elements may be applied when using blue illumination 80A for providing green and red illumination; when using white illumination 80B for enhancing green and red illumination and adjusting the illumination spectrum; and possibly when using blue and green illumination 80C (e.g., with blue and green LEDs in backlight units 300) for providing red illumination and enhancing red illumination and adjusting the illumination spectrum.

In any of the above-disclosed cases, assistant dye compounds 117 may be used as disclosed below (e.g., FIGS. 10A-E, 13A-B) to enhance any of the efficiency, FWHM, peak shape and/or white point of the illumination reaching RGB filters 220 and the illumination provided by LCD display 100. Assistant dye compounds 117 may be selected to have specified absorption and emission peaks and/or to have absorption curves and fluorescence curves which change the shape of illumination spectrum 80A and/or 80B and/or change the shape and intensity of illumination components in the transmission ranges of RGB filters 220. Two non-limiting examples for assistant dyes 117 are 5-FAM and 5-Carboxyfluorescein. Another non-limiting example of assistant dye 117 is HPTS; pyranine (8-Hydroxypyrene-1,3,6-Trisulfonic Acid, Trisodium Salt), having an absorption peak at shorter wavelengths than 5-FAM (e.g., at ca. 450 nm vs. 490 nm), with a similar emission peak at 520-530 nm (depending on embedding conditions). Other non-limiting examples of assistant dye 117 are rhodamine 12, rhodamine 101 from Atto-Tec® and perylene dye F300 from Lumogen®. Assistant dye compounds 117 may be integrated in any of disclosed films 130, 132, 134, 133, and/or in separate film(s).

### Examples for Assistant Dyes

Disclosed assistant dyes 117 are provided, which may be used to modify a received spectrum by absorption of radiation at some spectral regions to reduce their intensity and emission of radiation at other spectral regions to increase their intensity. Disclosed assistant dyes 117 may be used to convert radiation in the LCD display—possibly in combination with other assistant dyes disclosed herein.

In some embodiments, assistant dye compounds 117 may be represented by the following structure of Formula (A1):

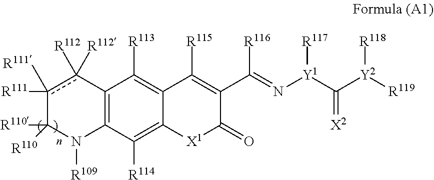

wherein
R¹⁰⁹ is H, alkyl, haloalkyl, heterocycloalkyl, cycloalkyl, aryl or benzyl;
R¹¹⁰ and R^{110′} are each independently H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹¹, R^{111′}, R¹¹² and R^{112′} are each independently absent, H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹³ is H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹⁴ is H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹⁵ is H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹⁶ is H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹⁷ is H, alkyl, haloalkyl, heterocycloalkyl, cycloalkyl, aryl or benzyl;
R¹¹⁸ and R¹¹⁹ are each independently H, alkyl, haloalkyl, heterocycloalkyl, cycloalkyl, aryl or benzyl;
X¹ is NR^{a}, S or O;
X² is NR^{a}, S or O
Y¹ is N or CR^{a};
Y² is N or CR^{a};
R^{a} is H, alkyl, haloalkyl, cycloalkyl, heterocycloalkyl, aryl or benzyl;
n is an integer between 0 and 3; and
wherein if there is a double bond between the carbons which are substituted by R¹¹¹, R^{111′}, R¹¹² and R^{112′}—then one of R¹¹¹ and R^{111′} is absent and one of R¹¹² and R^{112′} is absent.

In another embodiment, assistant dye compounds 117 may be represented by the following structure of Formula (A1a):

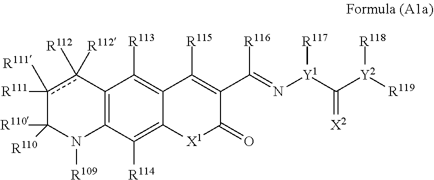

wherein
R¹⁰⁹ is H, alkyl, haloalkyl, heterocycloalkyl, cycloalkyl, aryl or benzyl;
R¹¹⁰ and R^{110′} are each independently H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹¹, R^{111′}, R¹¹² and R^{112′} are each independently absent, H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹³ is H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹⁴ is H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹⁵ is H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹⁶ is H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹⁷ is H, alkyl, haloalkyl, heterocycloalkyl, cycloalkyl, aryl or benzyl;
R¹¹⁸ and R¹¹⁹ are each independently H, alkyl, haloalkyl, heterocycloalkyl, cycloalkyl, aryl or benzyl;
X¹ is NR^{a}, S or O;
X² is NR^{a}, S or O
Y¹ is N or CR^{a};
Y² is N or CR^{a};
R^{a} is H, alkyl, haloalkyl, cycloalkyl, heterocycloalkyl, aryl or benzyl; and
wherein if there is a double bond between the carbons which are substituted by R¹¹¹, R^{111′}, R¹¹² and R^{112′}—then one of R¹¹¹ and R^{111′} is absent and one of R¹¹² and R^{112′} is absent.

In some embodiments, assistant dye compounds 117 may be represented by the following structure of Formula (A2):

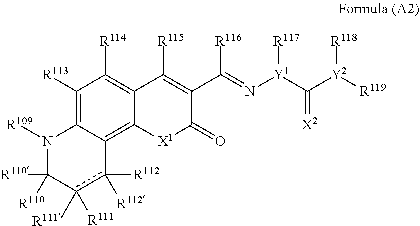

wherein
R¹⁰⁹ is H, alkyl, haloalkyl, heterocycloalkyl, cycloalkyl, aryl or benzyl;
R¹¹⁰ and R^{110′} are each independently H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹¹, R^{111′}, R¹¹² and R^{112′} are each independently absent, H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹³ is H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹⁴ is H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹⁵ is H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹⁶ is H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹⁷ is H, alkyl, haloalkyl, heterocycloalkyl, cycloalkyl, aryl or benzyl;
R¹¹⁸ and R¹¹⁹ are each independently H, alkyl, haloalkyl, heterocycloalkyl, cycloalkyl, aryl or benzyl;
X¹ is NR^{a}, S or O;
X² is NR^{a}, S or O
Y¹ is N or CR^{a};
Y² is N or CR^{a};
R^{a} is H, alkyl, haloalkyl, cycloalkyl, heterocycloalkyl, aryl or benzyl; and
wherein if there is a double bond between the carbons which are substituted by R¹¹¹, R^{111′}, R¹¹² and R^{112′}—then one of R¹¹¹ and R^{111′} is absent and one of R¹¹² and R^{112′} is absent.

In some embodiments, assistant dye compounds 117 may be represented by the following structure of Formula (A3):

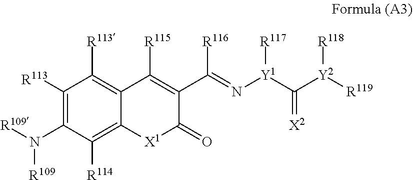

wherein
R¹⁰⁹ and R^{109′} are each independently is H, alkyl, haloalkyl, heterocycloalkyl, cycloalkyl, aryl or benzyl;
R¹¹³ and R^{113′} are each independently is H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹⁴ is H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹⁵ is H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹⁶ is H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹⁷ is H, alkyl, haloalkyl, heterocycloalkyl, cycloalkyl, aryl or benzyl;
R¹¹⁸ and R¹¹⁹ are each independently H, alkyl, haloalkyl, heterocycloalkyl, cycloalkyl, aryl or benzyl;
X¹ is NR^{a}, S or O;
X² is NR^{a}, S or O
Y¹ is N or CR^{a};
Y² is N or CR^{a}; and
R^{a} is H, alkyl, haloalkyl, cycloalkyl, heterocycloalkyl, aryl or benzyl.

In some embodiments, assistant dye compounds 117 may be selected from the group consisting of compounds A1-, A1-2, A1-3, A2-1, A2-2 and A3-1, represented by the following structures:

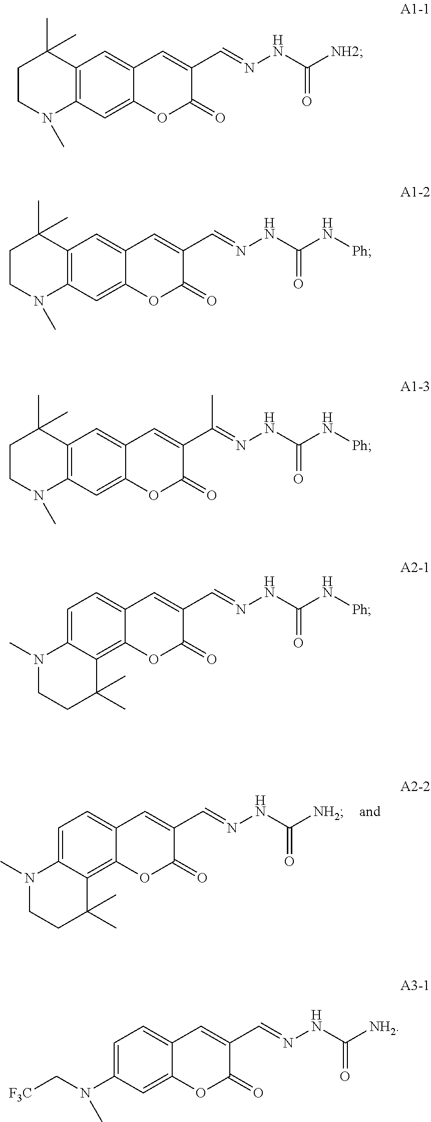 [SMILES: CN1CCC(C)(C)c2cc3cc(/C=N/NC(N)=O)c(=O)oc3cc21 ; CN1CCC(C)(C)c2cc3cc(/C=N/NC(=O)Nc4ccccc4)c(=O)oc3cc21 ; C/C(=N\NC(=O)Nc1ccccc1)c1cc2cc3c(cc2oc1=O)N(C)CCC3(C)C ; CN1CCC(C)(C)c2c1ccc1cc(/C=N/NC(=O)Nc3ccccc3)c(=O)oc21 ; CN1CCC(C)(C)c2c1ccc1cc(/C=N/NC(N)=O)c(=O)oc21 ; CN(CC(F)(F)F)c1ccc2cc(/C=N/NC(N)=O)c(=O)oc2c1]

In these five non-limiting examples, Compounds A3-1 and A1-3 absorb violet radiation and emits blue radiation, effectively converting radiation from the violet range to the blue range, while the other Compounds A1-, A1-2, A2-1 and A2-2 absorb blue radiation and emit green radiation, effectively converting radiation from the blue range to the green range, as illustrated by the data provided in the following Table 1.

TABLE 1
Absorption and emission wavelengths (in EtOH, ethanol),
quantum yield, and FWHM (Full width at half maximum
of the emission) for the exemplified compounds.

| | Absorbance | Emission | Quantum | |
|---|---|---|---|---|
| | EtOH | EtOH | yield | FWHM |
| Compound | [nm] | [nm] | [%] | [nm] |
| Compound A1-1 | 447 | 503 | 0.75 | 55 |
| Compound A1-2 | 446 | 510 | 0.72 | 55 |
| Compound A1-3 | 415 | 494 | 0.1 | 68 |
| Compound A2-1 | 443 | 510 | 0.74 | 55 |
| Compound A2-2 | 440 | 505 | 0.75 | 55 |
| Compound A3-1 | 408 | 473 | — | — |

In some embodiments, assistant dye compounds 117 may absorb light in the wavelength range of 350-800 nm. In one embodiment, the absorbance range is 350-500 nm. In another embodiment, the absorbance range is 400-500 nm. In another embodiment, compound 27 absorbs light at 408 nm (in Ethanol). In another embodiment, compound 28 absorbs light at 447 nm (in Ethanol). In another embodiment, compound 29 absorbs light at 446 nm (in Ethanol). In another embodiment, compound 30 absorbs light at 443 nm (in Ethanol). Each possibility represents a separate embodiment of this invention.

In some embodiments, assistant dye compounds 117 may emit light in the wavelength range of 400-800 nm. In one embodiment, the emission range is 400-550 nm. In another embodiment, compound 27 emits light at 473 nm (in Ethanol). In another embodiment, compound 28 emits light at 503 nm (in Ethanol). In another embodiment, compound 29 emits light at 510 nm (in Ethanol). In another embodiment, compound 30 emits light at 510 nm (in Ethanol). Each possibility represents a separate embodiment of this invention.

In some embodiments assistant dye compounds 117 may have a quantum yield (QY) of between 0.5-1. In one embodiment, the QY is 0.7-0.9. In another embodiment, compound 28 has a QY of 0.75. In another embodiment, compound 29 has a QY of 0.72. In another embodiment, compound 30 has a QY of 0.74. Each possibility represents a separate embodiment of this invention.

In some embodiments assistant dye compounds 117 may have a Full width at half maximum (FWHM) of emission being between 30-60 nm. In one embodiment, the FWHM is between 50-60 nm. In another embodiment, compound 28 has an FWHM of 55 nm. In another embodiment, compound 29 has an FWHM of 55 nm. In another embodiment, compound 30 has an FWHM of 55 nm. Each possibility represents a separate embodiment of this invention.

### Display Configurations

### Film Positions and Optional Patterning

FIGS. 2A-2J are high level schematic illustrations of configurations of digital display 100 with color conversion film(s) 130, according to some embodiments of the invention. Digital displays 100 are illustrated schematically as comprising a backlight unit 300 and a LCD panel 200, the former providing RGB illumination 120 to the latter.

Backlight unit 300 is illustrated schematically in FIG. 2A in a non-limiting manner as comprising a backlight source 400 (e.g., white LEDs 80B or blue LEDs 80A), a waveguide 420 with reflector(s) (the latter for side-lit waveguides), a diffuser 406, prism film(s) 355 (e.g., brightness enhancement film (BEF), dual BDF (DBEF), etc.) and polarizer film(s) 302, which may be configured in various ways. Films 130 may be applied at various positions in backlight unit 300 such as on either side (130A, 130B) of diffuser 406, on either side (130C, 130D) of at least one of prism film(s) 355, on either side (130E, 130F) of at least one polarizer film(s) 302, etc. In certain embodiments, film 130 may be deposited on any of the film in back light unit 300.

In certain embodiments, films 130 may be used to replace diffuser 406 and/or polarizer film 302 (and possibly prism film(s) 355), once appropriate optical characteristics are provided in films 130 as explained herein.

The location of film(s) 130 may be optimized with respect to radiation propagation in backlight unit 300, in both forwards (120A) and backward (120B) directions due to reflections in backlight unit 300. For example, optimization considerations may comprise fluorescence efficiency, energy efficiency, stability of rhodamine-based fluorescent (RBF) compounds 115 or other color conversion elements in film(s) 130, and so forth. As a non-limiting example, in the position of the lower film 130A, B (e.g., on diffuser 406) more radiation is expected to excite RBF compounds 115—increasing its conversion efficiency but increasing losses and reducing the durability of RBF compounds 115. In the position of the higher film 130E, F (e.g., on polarizer film 302) less radiation is expected to excite RBF compounds 115—reducing its conversion efficiency but reducing losses and increasing the durability of RBF compounds 115 and/or other color conversion elements in film(s) 130.

Some embodiments of displays 100 comprise a blue light source 80A (such as blue LEDs—light emitting diodes) with film(s) 130 configured to provide red and green components in RGB illumination 120, e.g., by using red-fluorescent RBF compound(s) (e.g., with silane precursor(s) such as PhTMOS (trimethoxyphenylsilane) and/or TMOS (trimethoxysilane) with fluorine substituents—see below) and green-fluorescent RBF compound(s) (e.g., with silane precursor(s) such as F₁TMOS (trimethoxy(3,3,3-trifluoropropyl)silane)—see below). It is emphasized that various silane precursor(s) 104 may be used with either red-fluorescent or green-fluorescent RBF compounds 115 as disclosed below.

The red and green fluorescent RBF compound(s) may be provided in a single film layer 133 or in multiple film layers 134, 132. The process may be optimized to provide required absorption and emission characteristics of RBF compounds in film 130, while maintaining stability thereof during operation of display 100. Similarly, film(s) 130 with either one or more color conversion elements (e.g., other fluorescent compounds, organic or inorganic, quantum dots etc.) may be integrated in display 100 in a similar way and according to respective considerations. In the following any of the mentioned RBF compound(s) may, in some embodiments, be replaced or augmented by other color conversion elements (e.g., other fluorescent compounds, organic or inorganic, quantum dots etc.).

Some embodiments of displays 100 comprise a white light source 80B (such as white LEDs) with film(s) 130 configured to provide red and green components in RGB illumination 120, e.g., by using red-fluorescent RBF compound(s) (e.g., with PhTMOS and/or TMOS with fluorine substituents as silane precursor(s)). The red fluorescent RBF compound(s) may be provided in a single film layer or in multiple film layers 134. The process may be optimized to provide required absorption and emission characteristics of RBF compounds in film 130, while maintaining stability thereof during operation of display 100. Red-fluorescent RBF compound(s) may be used to shift some of the yellow region (550-600 nm) in the emission spectrum of white light source 80B into the red region, to reduce illumination losses in LCD panel 200 while maintaining the balance between B and R+G in RGB illumination 120.

FIG. 2B illustrates in more details various films and elements in display 100 to which film 130 may be associated or which may be replaced by film 130 in some embodiments. LCD panel 200 is shown to include compensation films 204, 254, glass layers 206, 252, thin film transistors (TFT) 255, ITO (indium tin oxide) layers 258, 262, liquid crystal cell (LC) 261, RGB color filters 220, polarizer film 256 and protective film 266 (e.g., anti-glare, anti-reflection). FIG. 2B further illustrates typical illumination transmission in each layer and cumulatively, indicating ca. 40% loss in backlight unit 300 and 90% loss in LCD panel 200, the latter mainly resulting from RGB color filters 220 and polarizers 202, 302 in LCD panel 200 and backlight unit 300, respectively. One or more film(s) 130 may be attached to or replace any of various layers in backlight unit 300 and/or in LCD panel 200, depending on considerations of minimizing further illumination losses, film performance and lifetime of the fluorescent dyes (RBF compounds 115). As non-limiting examples, FIG. 2B illustrates schematically associating on one or more films 130 with any of diffuser and/or light guide 406, reflector 421, prism layer(s) 355, diffusers 360, 361, polarizers 83A, 83B (in either or both backlight unit 300 and LCD panel 200, respectively), LC 261, ITO 262 and/or color filters 220. It is emphasized that FIG. 2B merely provides a non-limiting example of a display configuration, and films 130 may be applied at various positions and any display configuration. It is noted that in various display configurations, disclosed layers may be to some extent re-ordered and modified. In following disclosed configurations, particularly polarizers and diffusers may be illustrated at different locations, reflecting the diversity of LCD designs.

In some embodiments, similar considerations may be used with respect to positioning of any type of color conversion film 130, which may comprise color conversion elements other than RBF compounds 115, such as organic (non-rhodamine-based) or inorganic fluorescent compounds, quantum dots etc. Various display 100 configurations may be provided, which optimize illumination loss with film parameters and lifetime of the color converting elements.

FIGS. 2C and 2D schematically illustrate some of the above considerations, by comparing display 100 (FIG. 2D) with color conversion film 130 in LCD panel 200 versus display 100 (FIG. 2C) with color conversion film 130 in backlight unit 300. The schematic illustrations depict the illumination intensity as I₀, and illumination components R, G, B as they are produced in the respective display. In display 100, color conversion film 130 in backlight unit 300 provides illumination at RGB, assuming in a non-limiting manner no loss on the conversion. In LCD panel 200, color filters 220 remove two of the three illumination components, leaving ca. 10% of the original illumination at each color component (see also FIG. 2B, illustrating a more realistic lower rate of less than 5% per color component). When placing color conversion film 130 in LCD panel 200 (e.g., as a patterned film 130, see e.g., schematic illustration of patterning 520 in FIG. 11), as illustrated for display 100 (FIG. 2D, assuming blue LED illumination), a blue component may be delivered directly to blue color filter 220 without color conversion or filtering, while R and G may be converted from corresponding blue component just before filters 220, so that that filters 220 pass most or all of the illumination they receive, which is wavelength-adjusted just before entering color filters 220—resulting in a much higher efficiency than in display 100 of ca. 30% of the original illumination at each color component (corresponding to 10-15% per color component in terms of FIG. 2B).

Such gain in efficiency may be achieved by some embodiments having any type of color conversion film 130, which may comprise color conversion elements other than RBF compounds 115, such as organic (non-rhodamine-based) or inorganic fluorescent compounds, quantum dots etc. Various display configurations may be provided which increase illumination use efficiency by positioning respective color conversion film 130 in LCD panel 200, before color filters 220. Some embodiments comprise respective LCD panels 200 having color conversion film 130 integrated therein and positioned before color filters 220 thereof, as well as corresponding displays 100.

FIG. 2E illustrates an example for configuration of film 130 folded into a corrugated (e.g., zig-zag folded) form, characterized by an overall length L, overall thickness d₁ and step d₂ between folds. Film 130 may be folded to increase the film thickness through which the illumination passes, without increasing the actual thickness of film 130 (formulated otherwise—to reduce the light flux per area of film 130). The folding may increase the lifetime of RBF compounds 115 in film or of any other comprise color conversion elements on which film 130 may be based, such as organic (non-rhodamine-based) or inorganic fluorescent compounds, quantum dots etc.

FIGS. 2F-2L are high level schematic illustrations of configurations of digital display 100 with color conversion film(s) 130, according to some embodiments of the invention. FIG. 2F illustrates, schematically, embodiments in which color conversion film 130 is positioned in backlight unit 300, e.g., between diffuser 406 and prism 355 or associated therewith, as disclosed above.

FIG. 2G illustrates, schematically, embodiments in which color conversion film 130 is positioned in LCD panel 200 between polarizer 202/258 and an analyzer film 262/256 (e.g., a corresponding polarizing film), e.g., between liquid crystal layer 261 and analyzer film 262/256 and below RGB color filter layer 220. In such configurations, with LCD panel 200 comprising, sequentially with respect to received illumination 120: polarizing film 202/258, liquid crystal layer 261, color conversion film 130, RGB color filter layer 220 and analyzer film 262/256—the position of color conversion film 130 may be optimized to provide maximal light conversion efficiency while retaining long life time (due to less radiation passing though film 130 after non-polarized illumination has been filtered out by polarizer 202/258) and maintaining the polarization of the illumination. The latter effect may be achieved by corresponding configuration of color conversion film 130 to maintain or even enhance the respective polarization, e.g., by aligning RBF compounds 115 during preparation of color conversion film 130, as disclosed herein. One or more color conversion film(s) 130 may be positioned in certain embodiments between polarizer 202/258 and liquid crystal layer 261.

It is noted that in both FIGS. 2F and 2G, LC module 210 may include color filter layer 220 (and possibly color conversion layer 130), in addition to LC film 261 and polarizers 262/256. It is noted that in both FIGS. 2F and 2G, LCD panel 200 may comprise LC module 210 and additional layers, which are not shown in these figures.

FIG. 2H illustrates, schematically, embodiments in which color conversion film 130 is positioned in LCD panel 200 after analyzer film 262/256 and below RGB color filter layer 220. In certain embodiments, RGB color filter layer 220 in LCD panel 200 may be positioned after analyzer film 262/256, and be preceded by color conversion film 130. In such configurations, with LCD panel 200 comprising, sequentially with respect to received illumination 120: polarizing film 202/258, liquid crystal layer 261, analyzer film 262/256, color conversion film 130, RGB color filter layer 220 and protective film 266. The position of color conversion film 130 may be optimized to provide maximal light conversion efficiency while retaining long life time (due to less radiation passing though film 130 after non-polarized illumination has been filtered out by polarizer 202/258). Polarization maintenance is not necessarily required in these embodiments, as color conversion film 130 is positioned after liquid crystal layer 261 and analyzer film 262/256. One or more color conversion film(s) 130 may be positioned in certain embodiments between analyzer film 262/256 and protective film 266. In certain embodiments, multiple films 130 may be used in display 100, e.g., combining embodiments illustrated in FIGS. 2F-2H, possibly with different films 130 which are configured each with respect to its position in display 100. In certain embodiments, color conversion film(s) 130 may be patterned with respect to a patterning of RGB color filter layer 220 to yield a spatial correspondence between film regions with R and G emission peaks and respective R and G color filters, as disclosed herein (see e.g., FIG. 2D). Color conversion film(s) 130 may comprise one or more layers, with corresponding red-fluorescent RBF compound(s) and green-fluorescent RBF compound(s) as disclosed herein. Color conversion film(s) 130 may comprise independent film(s) and/or corresponding layers applied onto any of the LCD panel components disclosed herein, according to their respective position in LCD panel 200. It is noted that in FIGS. 2F-2H, LCD panel 200 may comprise additional layers, which are not shown in the figures.

In certain embodiments, considerations for positioning color conversion film(s) 130 within LCD panel 200 may be carried out according to estimations of transmission of illumination, similar to the non-limiting example presented in FIG. 2B. The considerations may comprise minimizing radiation intensity passing through color conversion film(s) 130 with respect to the complexity of modifying LCD panel 200. Additional considerations may comprise reduction of parallax effects due to film thickness, which may be achieved by close association of film(s) 130 with color filters 220, applying at least part(s) of film(s) 130 as coatings on color filters 220 or on other films in LCD panel 200, and possibly providing barriers in film(s) 130 to limit stray light.

FIG. 2I is a high level schematic illustration of an intensity regulating mechanism implemented by a controller 212, according to some embodiments of the invention. Controller 212 may be configured to regulate transmission through LC module 210, e.g., by controlling LC layer 261 and/or polarizers 202/258/262/256) in relation to the intensity of fluorescence from color conversion film 130. For example, controller 212 may be configured to tune down transmission through LC module 210 when color conversion film 130 is fresh and provides a high level of fluorescence, and gradually tune up transmission through LC module 210 as color conversion film 130 degrades and provides less fluorescence. Such operation of controller 212 may be configured to provide a constant output from display 100, even within a given range of degradation of color conversion film 130 to increase the lifetime of display 100.

FIG. 2J is a high level schematic illustration of a fluorescence-intensifying section 150 with color conversion film 130, according to some embodiments of the invention. Section 150 may comprise optical elements 152 and optionally 154, configured to enhance red and green radiation by reflecting fluorescent radiation from green-fluorescent and red-fluorescent RBF compounds 115 (indicated schematically by the arrows) back in direction of color filters 220 (not illustrated). The distribution and density of green-fluorescent and red-fluorescent RBF compounds 115 in color conversion film 130 may be configured to take into account recurring fluorescence to provide the required white point parameters. Section 150 may be configured to pass the blue illumination component without reflections (attenuated only by the absorption by RBF compounds 115). For example, optical element 152 may comprise DBEF (Dual Brightness Enhancement Film) film(s) which may be configured to be transparent to blue light and reflective to red and green light. Optical element 154 may also comprise DBEF film(s) configured to be transparent to blue light and reflective to red and green light, to form some back and forth reflections of R and/or G light through color conversion film 130. Optical element 154 is optional in the sense that fluorescence-intensifying section 150 may comprise only optical elements 152 to enhance R and/or G light by simple reflection. In certain embodiments, fluorescence-intensifying section 150 may be also configured to enhance the degree of polarization of the illumination, by selectively reflecting (by optical element 152) and/or transmitting (by optical element 137) light with specified polarization properties, in particular red and green light with specified polarization properties. Fluorescence-intensifying section 150 may at least partly compensate for possibly loss of polarization by fluorescence of RBF compounds 115 in color conversion film 130. Fluorescence-intensifying section 150 may be positioned in either backlight unit 300 and/or LCD panel 200, and may be combined with any of the disclosed display configurations. As illustrated schematically, fluorescence-intensifying section 150 may be positioned in various, one or more positions in BL 300 and/or in LCD panel 200 (see also FIG. 1A). Advantageously, fluorescence-intensifying section 150 may be configured to reduce stray light, compensate for absorption and/or enhance polarization of light passing through color conversion film 130.

In certain embodiments, enhancements may be applied to color conversion film 130 integrated in backlight unit 300 and/or in LCD panel 200. For example, a short-pass reflector (SPR) layer (see e.g., layer 152 in FIG. 2L) may be positioned before color conversion film 130 to reflect backward fluorescent emission of RBF compounds 115 into the forward direction, to prevent absorption loss of the backward fluorescent emission. It is noted that SPR layer 152 may be implemented as any of, e.g., single-edge short-pass dichroic beam splitter(s), bandpass filter(s) and/or blocking single-band bandpass filter(s) or their combinations. In certain embodiments, a layer may be positioned after color conversion film 130 to enhance the fluorescent output of color conversion film 130 by directing more radiation through it; to reduce stray fluorescent emission and possibly to reduce cross talk between RGB color filters 220 (see also crosstalk-reducing layer 160 disclosed below). In certain embodiments, possible polarization scrambling by film 130 may be compensated by a layer positioned before or after film 130, such as a thin analyzer (polarizer) layer 258.

FIGS. 2K and 2L are high level schematic illustrations of patterned color conversion films 130 with a matrix-like crosstalk-reducing layer 160, according to some embodiments of the invention. FIG. 2K illustrates schematically a cross section through a part of LCD panel 200, between polarizer 202 and analyzer 260 of embodiments similar to the illustrated in FIG. 2G. It is noted that in various display configurations (see e.g., FIG. 2B), disclosed layers may be to some extent re-ordered and modified. In following disclosed configurations, particularly polarizers and diffusers may be illustrated at different locations, reflecting the diversity of LCD designs.

In certain embodiments, color conversion film 130 may be patterned and attached to or adjacent to RGB color filters layer 220. Regions of color conversion film 130 which are adjacent to B (blue) color filter regions of layer 220 may be devoid of RBF compounds 115 and pass all the blue light (see also FIG. 2D); regions of color conversion film 130 which are adjacent to G (green) color filter regions of layer 220 may comprise only green-fluorescent RBF compounds 115 to convert blue light to green light; and regions of color conversion film 130 which are adjacent to R (red) color filter regions of layer 220 may comprise both green-fluorescent and red-fluorescent RBF compounds 115 to convert blue light to green light and green light to red light, respectively. The film stack comprising patterned color conversion film 130, color filters layer 220 and possibly liquid crystal (LC) layer 261, polarizer 202 and analyzer 260 (indicated as an LC module 210)—may be produced or processed jointly to achieve exact alignment of patterned color conversion film 130 and color filters layer 220.

Color conversion films 130 may have a crosstalk-reducing layer 160 embedded therein (see also FIG. 2M below), and/or patches of color conversion film 130 may be incorporated within the structural framework of crosstalk-reducing layer 160. Color conversion film 130 with crosstalk-reducing layer 160 may be patterned to comprise compartments of film 130 with green-fluorescent RBF compounds 115, denoted 130 (115G)—before the G filter regions of RGB filter 220, compartments of film 130 with both red-fluorescent and green-fluorescent RBF compounds 115, denoted 130 (115R) and 130 (115G), respectively—before the R filter regions of RGB filter 220 and compartments with blue or no film 130 (e.g., possibly blue emitting film “B”, a diffuser and/or a void, as explained below) before the B filter regions of RGB filter 220.

FIG. 2L illustrates schematically a cross section through a part of LCD panel 200, with additional optical elements configured to optimize the LCD output and the radiation movement through the LC panel. For example, SPR layer 152 may be used before layer 130 to recycle backscattered fluorescent light and possibly to increase blue transmission by configuration in the respective polarization; and optical elements 262, 264 may be used to control radiation after layer 130. For example, optical elements 264 may comprise diffuser or concave micro lens configured to correct possible spatial distribution differences in illumination between the B, R and G component from film 130 and filters 220 (e.g., possibly correcting deviations introduced be film 130).

Optical elements 264 may comprise, in addition or in place of analyzer 260, and possibly integrated in protective layer 266, optical elements configured to reflect back and/or absorb ambient light, a black matrix with micro lenses to further improve the LCD output. In certain embodiments, thin analyzer 258 may be positioned before SPR layer 152 to enhance the degree of polarization of the radiation reaching film 130, optionally to compensate for possible polarization scrambling in film 130. Thin analyzer 258 and SPR layer 152 (illustrated as stack 259) may be replaced by (main) analyzer 260, a glass substrate and SPR layer 152 in alternative embodiments of stack 259.

Certain embodiments comprise an integrated and patterned layer of color conversion film(s) 130, RGB color filters 220 and crosstalk-reducing layer 160, comprising the structural framework configured to reduce cross-talk between patterned pixels of the integrated layer.

FIG. 2M provides a schematic cross section view of a part of LCD panel 200 as well as a perspective view of color conversion films 130 with crosstalk-reducing layer 160, showing the top compartments thereof (130 (115G) of the red compartments are not visible in the image, see in FIGS. 2K, 2L). In non-limiting examples, layer 160 may have a honeycomb structure, a rectangular structure or any other structure designed to correspond to patterns of color filters 220 and/or to patterns of color conversion film 130 disclosed above. The combination of color conversion films 130 and crosstalk-reducing layer 160 may be implemented by a range of technologies, such as deposition methods, photolithography, solution-based coating methods and/or by producing a film (such as a white film, a black film, a reflective film etc.) with holes by the corresponding color-conversion materials (patches of film 130 with respective RBF compounds 115). Layers 130, 160 may be positioned next to LC layer 261 and/or after analyzer 87 (see e.g., FIGS. 2G, 2H, respectively), depending on the level pf polarization layers 130, 160 are configured to provide.

FIG. 2M further illustrates patterned color conversion film 130 with a matrix-like crosstalk-reducing layer 160 in an above-LC configuration, according to some embodiments of the invention. Collimated illumination 120 may be configured to enable maintaining the direction of illumination exiting the LC module as it propagates through color conversion film 130 to color filters 220 and exits display 100—to achieve a low level of blurring and high efficiency. FIG. 2M is a schematic cross section through a part of LCD panel 200, including polarizer 202, LC layer 210, polarizer (analyzer) 262, and patterned color conversion film 130 and color filters layer 220 positioned above polarizer (analyzer) 262.

FIGS. 2N and 2S are high-level schematic illustrations of LCD panel 200 comprising the color conversion and filtering layer above the LC module, with a top optical-elements array 222, according to some embodiments of the invention. The color conversion and filtering layer may comprise separate color conversion layer 130 and color filters layer 220 or integrated color conversion and filtering layer 230 as shown in FIGS. 2O and 2P below. LCD panel 200 may comprise top optical-elements array 222 having e.g., a micro-lens array (FIG. 2N), which is placed above color filters 220 and configured to increase the brightness and radiance of LCD 100 at the center of a vertical viewing direction. LCD panel 200 may comprise top optical-elements array 222 having optical elements such as lenslets, encapsulated within a transparent material (typically having a lower refractive index than the lenslets), as illustrated schematically in FIG. 2S, providing a flat optical element which is placed above color filters 220 and configured to increase the brightness and radiance of LCD 100 at the center of a vertical viewing direction.

FIG. 2N further illustrates schematically blue diffuser elements 161, which may be applicable to any of the embodiments disclosed herein, configured to provide a similar spatial distribution of blue light as the red and green light spatial distributions, which are affected by color conversion elements 130R and/or 130G. In certain embodiments, top optical-elements array 222 may comprise optical elements (e.g., micro-lenses) only over blue sub-pixels (in addition or in place of blue diffuser elements 161) to equalize the light spatial distributions of R, G and B light.

FIGS. 2O-2Q are high-level schematic illustrations of apart of LCD panel 200, according to some embodiments of the invention. FIG. 2P is a schematic cross section view, FIG. 2O is a schematic side and perspective view. In certain embodiments, crosstalk-reducing layer 160 and color conversion layer 130 may be integrated into single patterned color conversion film 133 and possibly further integrated with color filters layer 220 into a single layer 230 configured to perform both functions of color conversion and filtering. Layer 230 may be pixelated in any pattern of pixels and subpixels, and may have regions B, G+130G and R+130R (possibly with additional colors, e.g., yellow) configured to provide blue, green and red light from collimated blue illumination 120, through color conversion and color filtering. Corresponding concentrations and amounts of absorptive and fluorescent dyes may be produced into the compartments of layer 230 according to the principles disclosed herein, possibly integrated in a production process which is similar to the current process of producing color filters layer 220. Supporting elements and/or matrix-like crosstalk-reducing layer 160 may be part of layer 230 to maintain collimation of the provided light and minimize light stray. Integrated layer 230 may comprise any of color conversion layer 130, RGB filters layer 220 and crosstalk-reducing layer 160.

FIG. 2P is a high level schematic illustration of an integrated layer 230 of patterned color conversion film 130 with RGB color filters 220, according to some embodiments of the invention. In certain embodiments, one or more of RGB color filters 220 may be configured to comprise red-fluorescent and/or green-fluorescent RBF compounds 115 and/or assistant dyes 117 and be configured as respective integrated RGB color filters 230.

As illustrated e.g., in FIG. 2P, certain embodiments comprise LCD 100 comprising backlight unit 300 configured to provide illumination 120 and LCD panel 210 receiving illumination radiation from backlight unit 300 and comprising, sequentially with respect to the received illumination: polarizing film 202, 258, liquid crystal layer 261, analyzer film 262, integrated RGB color filter layer 220 which is integrated with color conversion film 130 (possibly patterned), and protective layer 266, possibly with additional analyzer film 262 between integrated RGB color filter layer 220 and protective layer 266—as illustrated e.g., in FIGS. 2K-2M. Integrated RGB color filter layer 220 may comprise rhodamine-based fluorescent (RBF) compounds 115 selected to absorb illumination from backlight unit 300 and have an R emission peak and a G emission peak.

Integration of color filters 220 with color conversion layer 130 may simplify the design of display 100 and enhance its efficiency (e.g., reduce losses, further reducing stray light and increasing the efficiency of utilization of illumination 120). In certain embodiments, illumination 120 may comprise blue illumination 80A and integrated RGB color filter layer 220 may comprise RBF compounds 115 having the R emission peak and the G emission peak. In certain embodiments, illumination 120 may comprise white illumination 80B and integrated RGB color filter layer 220 may comprise RBF compounds 115 having the R emission peak and/or the G emission peak configured to provide red and/or green color enhancement, respectively. In certain embodiments, illumination 120 may comprise blue and green illumination 80C and integrated RGB color filter layer 220 may comprise RBF compounds 115 having the R emission peak and/or the G emission peak configured to provide red color conversion and possibly red and/or green color enhancement, respectively. In any of the embodiments, assistant dyes 117 may be further integrated in integrated RGB color filter layer 220 and/or possibly used as separate color conversion elements 117.

In certain embodiments, the efficiency of illumination may be determined by a large number of parameters, such as spectrum overlap between illumination 120 from backlight unit 300 and absorption ranges of color conversion and assistant dyes 115, 117 respectively, transmission and reflection parameters in the spectral range of optical elements in LCD panel 210 (e.g., optical elements 152 and optionally 154 illustrated in FIG. 2J), quantum yields of the dyes and recycling efficiency of the backscattered fluorescent light; and spectrum overlap between the modified spectrum and color filters 220, e.g., spectrum overlap between the emission spectra of color conversion and assistant dyes 115, 117 respectively, and color filters 220, residual illumination after color conversion, and spatial considerations such as angular dependency of fluorescent radiation, and of optical elements in LCD panel 200. Optimization of color conversion and assistant dyes 115, 117 respectively, of dye integration in color filters 220, of spectrum shaping (see below) and of crosstalk-reducing layer 160 may be carried out with respect to individual color ranges and specified required gamut parameters.

FIG. 2Q further illustrates schematically red and/or green diffuser elements 161A, which may be applicable to any of the embodiments disclosed herein, configured to regulate the spatial distribution of red and/or green light, respectively, possibly to compensate for effects of color conversion elements 130R and/or 130G, respectively. In certain embodiments, blue diffuser elements 161 may be applied together with red and/or green diffuser elements 161A. Any of the embodiments may be configured to equalize the light spatial distributions of R, G and B light.

In certain embodiments, color conversion film 130 may be patterned and attached to or adjacent to RGB color filters layer 220. Regions of color conversion film 130 which are adjacent to B (blue) color filter regions of layer 220 may be devoid of color conversion compounds and pass all the blue light; regions of color conversion film 130G which are adjacent to G (green) color filter regions of layer 220 may comprise only green color conversion compounds, such as green-fluorescent rhodamine-based compounds disclosed in U.S. Patent Publication No. 2018/0057688, included herein by reference in its entirety, to convert blue light to green light; and regions of color conversion film 130R which are adjacent to R (red) color filter regions of layer 220 may comprise both green and color conversion compounds such as green-fluorescent and red-fluorescent rhodamine-based compounds disclosed in U.S. Patent Publication No. 2018/0057688 and U.S. Pat. No. 9,771,480, included herein by reference in their entirety, to convert blue light to green light and green light to red light, respectively.

Color conversion films 130 may comprise crosstalk-reducing layer 160 embedded therein (patterned in squares, hexagons, or other shapes), and/or patches of color conversion film 130 may be incorporated within the structural framework of crosstalk-reducing layer 160. Color conversion film 130 with crosstalk-reducing layer 160 may be patterned to comprise compartments 130G of film 130 with green color conversion compounds adjacent and before the G filter regions of RGB filter 220, compartments 130R, 130G (possibly combined or integrated) of film 130 with both green and red color conversion compounds adjacent and before the R filter regions of RGB filter 220 and compartments with blue or no film 130 (e.g., possibly blue emitting film, a diffuser and/or a void) adjacent and before the B filter regions of RGB filter 220.

In certain embodiments, additional layers may be added, such as short-pass reflector (SPR) layer(s) to recycle backscattered fluorescent light and possibly to increase blue transmission by configuration in the respective polarization, optical elements configured to control radiation after color conversion layer 130 such as diffuser(s) or concave micro lenses configured to correct possible spatial distribution differences in illumination between the B, R and G component from color conversion film 130 and filters 220, to reflect back and/or absorb ambient light, to further improve the LCD output e.g., using a black matrix with micro lenses, etc. In certain embodiments, a thin analyzer layer may be used as polarizer (analyzer) 262 to enhance the degree of polarization of the radiation reaching color conversion film 130, optionally to compensate for possible polarization scrambling therein.

FIG. 2R is a high level schematic illustration of patterned color conversion films 130 with a layer 117 of assistant dyes, according to some embodiments of the invention. Layer 117 of assistant dyes may be patterned, possibly with different assistant dyes associated with each of R, G and B filters 220, indicated schematically as assistant dye layers 117(R, G, B). In certain embodiments (not shown), assistant dye layers 117 may be integrated in one or more of patterned color conversion film(s) 130.

In certain embodiments, an illumination efficiency calculation may be used to adjust the relative amounts of illumination in each spectral range (e.g., R, G, B ranges). First, color conversion factors may be adjusted to provide relative amounts of R, G, B illumination reaching color filters 220 (e.g., green and red color conversion for blue illumination 80A, red color conversion for blue and green illumination 80C), second, color conversion dyes (and possibly assistant dyes) may be provided to adjust the illumination spectrum and fine tune the relative amounts of R, G, B illumination reaching color filters 220 (e.g., red and green enhancement for blue illumination 80A, red and green enhancement for white illumination 80B, red and possibly green enhancement for blue and green illumination 80C). Third, conversion efficiencies and adjustment efficiencies may be calculated together with efficiency figures of other components to adjust the relative intensities of R, G, B illumination provided by LCD display 100. For example, red and green enhancements may be configured to compensate for higher losses through red and green conversion films and possibly for higher losses for R illumination (due to double conversion—to green and then to red) than for G illumination (see also FIGS. 1D and 2J).

In certain embodiments, assistant dye(s) may comprise phosphorous compound(s) selected to convert blue illumination 80A to illumination at longer wavelengths, as an assistant component (e.g., in association with R color filters 220 as 117R).

In the case of blue illumination 80A which is used with quantum dots 76, red-fluorescent and/or green-fluorescent RBF compounds 115 and/or assistant dyes 117 may be used to enhance any of the efficiency, FWHM, peak shape and/or white point of the illumination reaching RGB filters 220 and the illumination provided by LCD display 100 (FIG. 1D). Red-fluorescent and/or green-fluorescent RBF compounds 115 and/or assistant dye compounds 117 may be selected to have specified absorption curves and fluorescence curves which change the shape of illumination spectrum 80A after it is modified by quantum dots 76 and/or change the shape and intensity of illumination components in the transmission ranges of RGB filters 220. In particular, red-fluorescent and/or green-fluorescent RBF compounds 115 and/or assistant dye compounds 117 may be selected to correct symmetry issues in the transmission ranges of RGB filters 220 which are prevalent when using certain color conversion technologies (see e.g., WIPO Publication No. WO 2017/085720 and U.S. Pat. No. 9,868,859, incorporated herein by reference in their entirety).

As disclosed above and illustrated in FIG. 2S, a flat optical element 222 may be placed above color filters 220 and configured to increase the brightness and radiance of LCD 100 at the center of a vertical viewing direction. Flat optical element 222 may comprise sub elements 224A such as micro lenses (shown schematically) embedded within transparent surface 224B. Optical element 222 may be configured to control the spatial intensity pattern of the radiation emitted by display 100.

FIG. 3A is a high-level schematic layered view of LCD 100 having collimated backlight unit 300, according to some embodiments of the invention. Analogously to FIGS. 1C and 2M-2S, FIG. 3A illustrates, sequentially, collimated backlight unit 300 providing collimated illumination 120, LC layer 210 with polarizers 202, 262, color conversion film 130 and color filter layer 220.

Backlight unit 300 may comprise at least one illumination source 80 configured to provide illumination, at least one cavity 110 (shown schematically) configured to receive the provided illumination from illumination source(s) 80, and an array of optical elements 116 (shown schematically) configured to collimate illumination exiting from respective cavities 110, to provide collimated illumination 120. Cavity 110 may be produced in different ways, possibly using internal reflective coatings the bottom, and possibly parts of the sides of cavities 110 from within, such as metallic coating, white coatings, highly reflective coatings such as Spectralon®, mirror coatings, possibly narrow-band mirrors (such as dielectric mirrors) or laser line mirrors, etc. In certain embodiments, the internal reflectivity of cavities 110 may be very high only over a narrow spectral band-width which corresponds to the wavelength band of illumination source(s) 80. In certain embodiments, cavities 110 are not internally reflective in any part thereof.

In certain embodiments, illumination sources 80, cavities 110 and optical elements 116 may be arranged in a light source layer 400 and/or may be arranged into illumination units 122, each comprising one illumination source 80, one cavity 110 and one optical element 116. It is noted that other embodiments may comprise different numeral ratios between the elements. It is further noted that the sizes of illumination units 122 in respective embodiments may be optimized with respect to illumination properties, possibly unrelated to LCD panel parameters such as pixel parameters.

FIGS. 3B and 3C are high-level schematic illustrations of illumination units 122 with designed serrated lens 111 and an additional lens 116 providing collimated illumination 120, according to some embodiments of the invention.

In certain embodiments, designed optical element 111 such as a serrated lens, may be configured to deliver radiation from illumination source(s) 80 to optical elements 116 to be collimated after optical elements 116 as collimated illumination 120. In certain embodiments, the design of optical element 111 may simulate illumination from a point source such as pinpoint openings 114 of internally reflective cavities 110 disclosed below. Designed optical element 111 may be molded onto illumination source(s) 80 and/or illumination source(s) 80 may be positioned within designed optical element 111, e.g., during a molding process thereof. Designed optical element 111 may be further configured to provide effective heat dissipation from illumination source(s) 80. In certain embodiments, designed optical element 111 may be configured to deliver collimated radiation 80 without need for additional optical elements 116.

Optionally, illumination units 122 may further comprise a reflector 111A configured to reflect radiation towards the LCD panel and possibly support lens 111 mechanically.

It is noted that FIGS. 3A and 4-6B are not drawn in correct proportions, as typically the dimensions of illumination sources 80 are several orders of magnitude smaller than the dimensions of cavities 110 and optical elements 116.

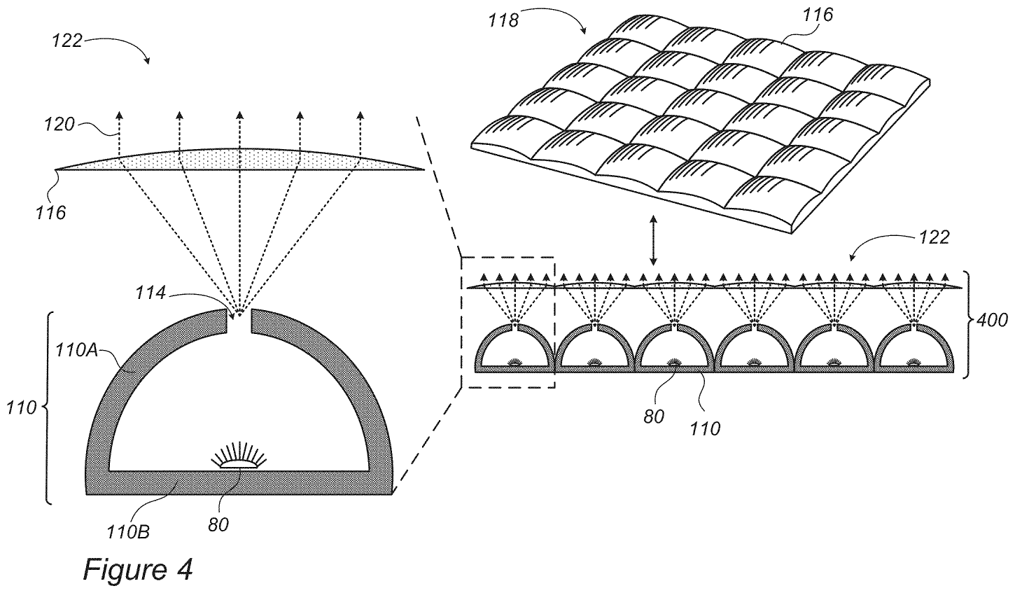

In certain embodiments, such as illustrated in FIGS. 4, 5A-5E and 6A-6B, illumination sources 80 (shown with supporting structure), internally reflective cavities 110 and optical elements 116 may be arranged in a light source layer 400 and/or may be arranged into illumination units 122, each comprising one illumination source 80, one internally reflective cavity 110 and one optical element 116. It is noted that other embodiments may comprise different numeral ratios between the elements, as exemplified below. It is further noted that the sizes of illumination units 122 in respective embodiments may be optimized with respect to illumination properties, possibly unrelated to LCD panel parameters such as pixel parameters.

It is noted that in any of FIGS. 4, 5A-5J and 6A-6B, internally reflective cavities 110 may be internally coated, lined and/or produced from any type of reflective coating or material, such as metallic coatings, white coatings, highly reflective coatings such as Spectralon®, mirror coatings, possibly narrow-band mirrors (such as dielectric mirrors) or laser line mirrors, etc.

FIG. 4 is a high-level schematic illustration of light source layer 400 and illumination units 122 with array 118 of optical elements 116, configured to provide collimated backlight illumination 120, according to some embodiments of the invention. In the non-limiting illustrated example, array 118 of optical elements 116 may comprise array 118 of lenslets 116, each lenslet 116 positioned to have a corresponding pinpoint opening 114 at its focal point and each lenslet 116 configured to collimate illumination exiting from corresponding pinpoint opening 114.

In certain embodiments, pinpoint openings 114 may be adjacent to the focal points of optical elements 116, or remote therefrom. In certain embodiments, optical elements 116 may comprise one or more grating(s) configured to collimate radiation from pinpoint openings 114. It is noted that in certain embodiments, LCD 100 may comprise light source layer 400 without optical elements 116.

Advantageously, internally reflective cavities 110 may be designed to effectively disperse heat generated by illumination source(s) 80 such as LEDs (light emitting diodes). In certain embodiments, materials of cavities 110 may be selected to provide effective heat dispersion. In certain embodiments, cooling means may be associated with cavities 110 and/or with illumination source(s) 80.

The deigns presented in FIGS. 3A-3C, 4, 5A-5J and 6A-6B may be modified and optimized with respect to illumination source(s) 80 (concerning parameters such as wavelength, power, size, type etc.), with respect to optical elements 116 (concerning parameters such as their presence, type, optical configuration, dimensions and pitch, coatings etc.), and with respect to cavities 110 (concerning parameters such as dimensions, form and design, pinhole shape and size, internal coatings, spacing or pitch between the cavities etc.). Further optimization may be carried out with respect to overall optical performance (at the LCD level), illumination-use efficiency and other parameters such as dimensions, complexity and costs.

In certain embodiments, multiple illumination source(s) 80 may be positioned within respective multiple internally reflective cavities 110, one illumination source 80 in each internally reflective cavity 110. Internally reflective cavities 110 may be configured to have any of a range of shapes, possibly determined by efficiency and production configurations. As a non-limiting example, FIG. 4 illustrates dome shaped internally reflective cavities 110, having reflective domes 110A and reflective bases 110B, with pinpoint openings 114 at the tops of respective domes 110A. In certain embodiments, internally reflective cavities 110 may have a common base (e.g., corresponding to bases 110B) on a plane parallel to the plane of pinpoint openings 114, and possibly parallel to array 118 of optical elements 116 such as array 118 of lenslets 116.

In certain embodiments, internally reflective cavities 110 may be triangularly shaped, with bases 110B and pinpoint openings 114 at the triangle tops (not shown). In certain embodiments, internally reflective cavities 110 may be box shaped, with bases 110B and pinpoint openings 114 at the box tops, on a plane parallel to bases 110B.

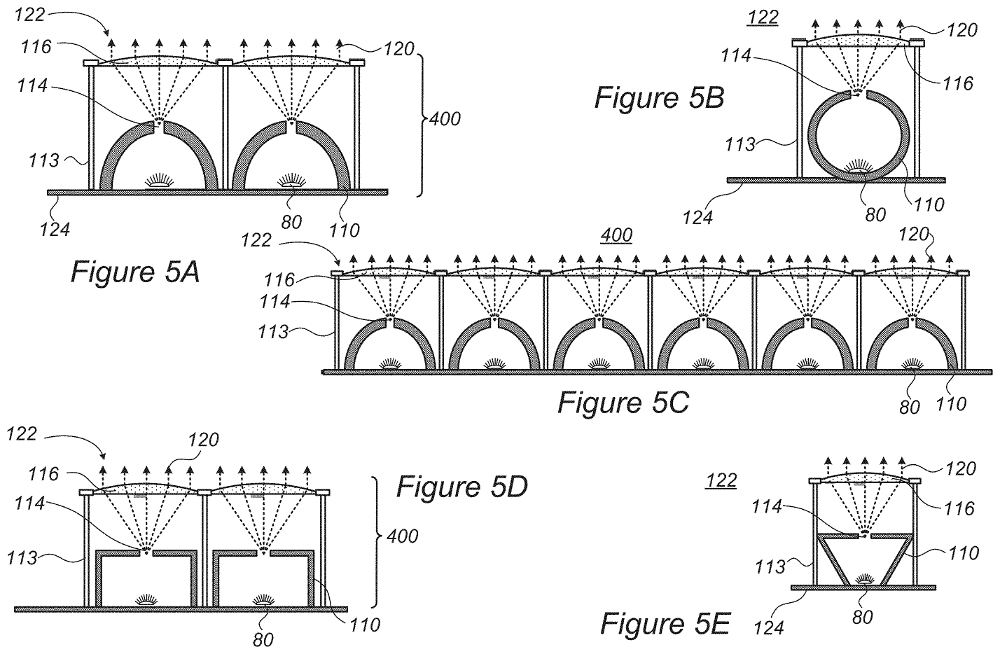

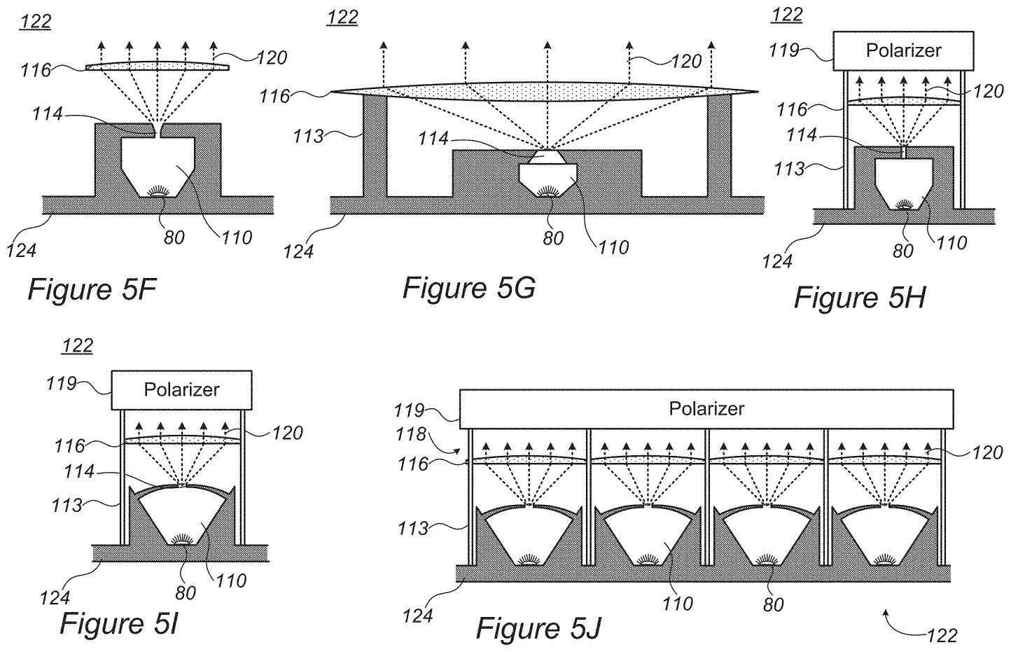

FIGS. 5A-5J are high-level schematic illustrations of illumination units 122 and light source layers 400, according to some embodiments of the invention. Various embodiments comprise internally reflective cavities 110 having different shapes (e.g., dome-shaped in FIGS. 5A and 5C, spheroid or ellipsoid in FIG. 5B, rectangular, or box-shaped, in FIG. 5D, triangular in FIG. 5E and various frustal forms such as truncated cones, truncated pyramids or related forms, illustrated schematically in FIG. 5F-5J); various configurations of supports 113 configured to mechanically stabilize light source layers 400 (and/or illumination units 122), supporting, e.g., each illumination unit 122 (FIGS. 5A, 5B, 5C, 5D) or groups of illumination units 122 (FIG. 5C). In certain embodiments, supports 113 may be configured to fixate a configured position of array 118 of optical elements 116 with respect to pinpoint openings 114 to optimize performance of backlight unit 300 and/or LCD 100. Heat dissipation elements 124 may be configured and positioned to remove heat from illumination source 80, e.g., along bases 110B of illumination units 122 (see FIG. 4). Pinpoint openings 114 may be designed to have different shapes, such as points (e.g., as illustrated in FIGS. 5A-5E, 5I and 5J), elongated tubes (e.g., as illustrated in FIGS. 5F and 5H) and/or cone or frustum shaped (e.g., as illustrated in FIG. 5G), configured to optimize illumination units 122 with respect to the collimation of illumination provided thereby, in cooperation with optical elements 116. In certain embodiments, a polarizer 119 may be part of illumination units 122 and configured to regulate collimated illumination using specified polarization directions, and possibly increase the degree of collimation of illumination 120 by removing less collimated radiation in different polarization direction(s). In certain embodiments, polarizer 119 may be configured to supplement or even replace polarizer 202 in LCD panel 200. Supports 113 may be further configured to support polarizer 119, which may be common to multiple illumination units 122, possibly to the whole of light source layer 400. Polarizer 119 may be set and be control for each one or groups of illumination units 122 (see FIGS. 5I, 5J, respectively) or one polarizer 119 may be provided for whole of light source layer 400. Supports 113 may be further configured to stabilize a distance between polarizer 119 and array 118 of lenslets 116.

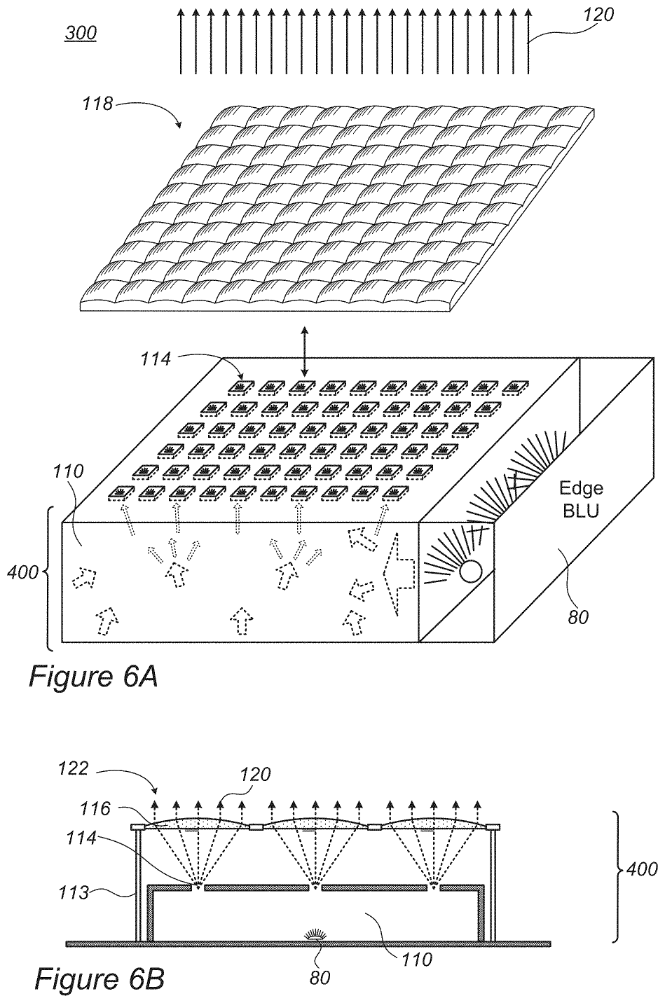

FIG. 6A is a high-level schematic illustration of light source layer 400 with lateral illumination source(s) 80 and common internally reflective cavity(ies) 110 having multiple pinpoint openings 114, configured to provide collimated backlight illumination 120, according to some embodiments of the invention. At least one illumination source 80 may be positioned on an edge of backlight unit 300, in optical communication with one or more internally reflective cavity 110, which may comprise a plurality of pinpoint openings 114. Array 118 of optical elements 116 (e.g., lenslets) may be set in parallel to one or more internally reflective cavity 110 and/or in parallel to the plane of pinpoint openings 114 (e.g., with pinpoint openings 114 at the focal points of lenslets 116) to collimate illumination delivered by lateral illumination source(s) 80 through one or more internally reflective cavity 110 (indicated as internal broken arrows) and out through pinpoint openings 114. In certain embodiments, one or more internally reflective cavity 110 may comprise single internally reflective cavity 110 with all pinpoint openings 114.

FIG. 6B is a high-level schematic illustration of light source layer 400 with multiple pinpoint openings 114 and associated optical elements 116 (e.g., lenslets) for each illumination source 80 and internally reflective cavity 110, configured to provide collimated backlight illumination 120, according to some embodiments of the invention. Multiple illumination units 122 may each comprise multiple pinpoint openings 114 and associated optical elements 116 (e.g., lenslets) for each illumination source 80 and internally reflective cavity 110.

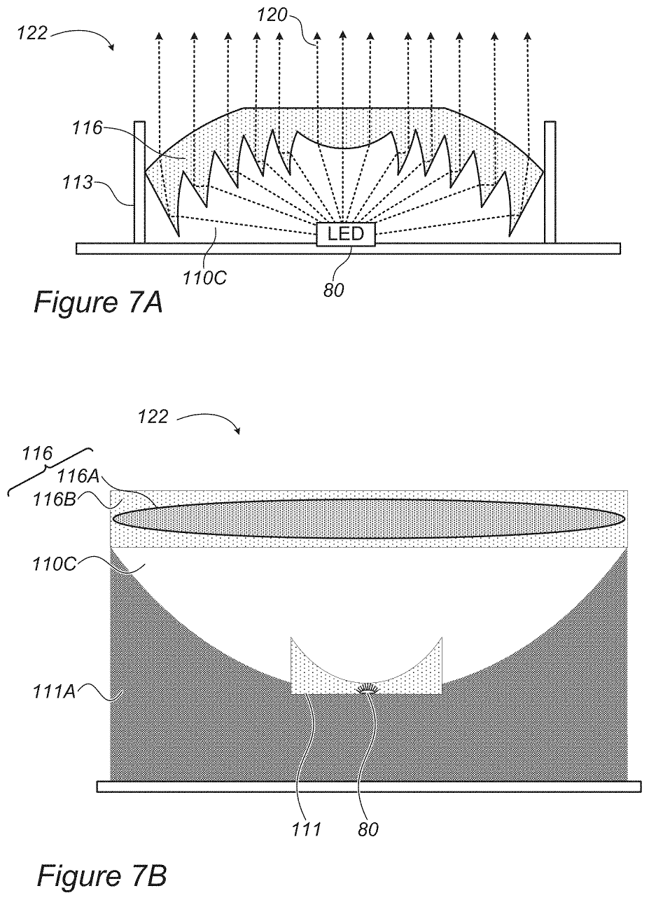

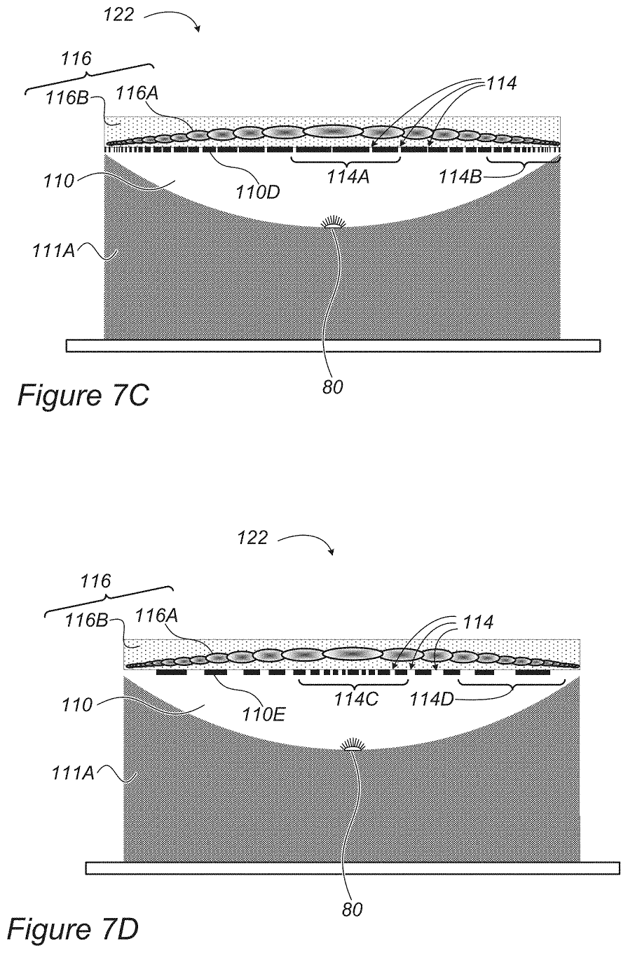

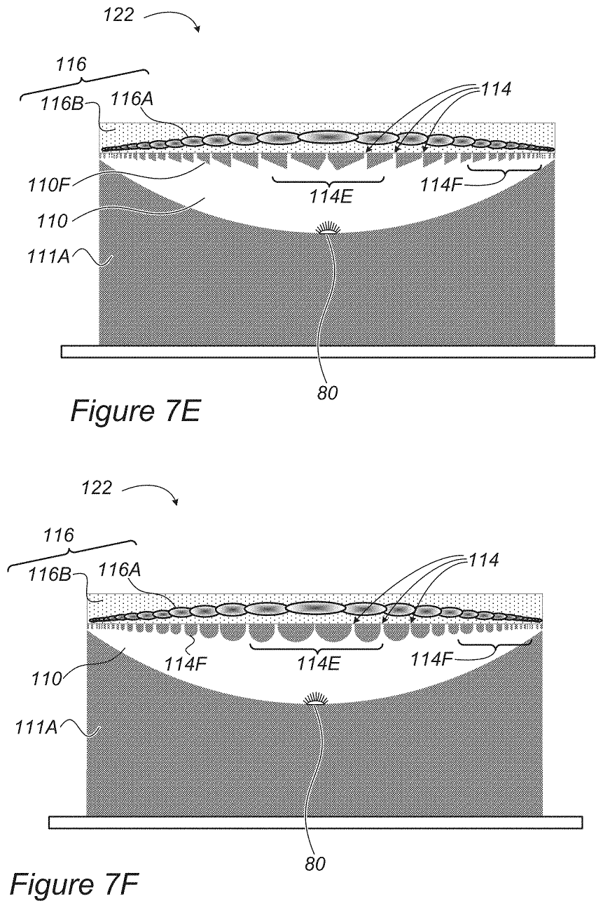

FIGS. 7A-7F are high-level schematic illustrations of illumination units 122, according to some embodiments of the invention.

In certain embodiments, a designed optical element 116 such as a serrated lens (see e.g., FIG. 7A), may be configured to deliver collimated radiation 120 by collimating radiation from illumination source(s) 80, possibly without any additional optical elements such as lenslets. In certain embodiments, a cavity 110C between illumination source(s) 80 and designed optical element 116 may be at least partly internally reflective, e.g., having a base reflector (not shown). Designed optical element 116 may be molded onto supports 113 of illumination units 122, e.g., during a molding process thereof.

In certain embodiments, a concave lens 111 or another designed optical element 111 may be configured to regulate the radiation distribution within cavity 110C, which may partly reflective (e.g., using reflector 111A, see FIG. 7B). Cavity 110C may have top reflective member 110A (e.g., a flat surface, a dome etc., see e.g. FIGS. 4 and 5A-6B) or may be defined directly by optical element(s) 116, possibly comprising sub elements 116A such as micro lenses within transparent surface 116B. Designed optical element 111 may be configured to define the dispersion of radiation within cavity 110C to optimize collimated illumination 120. For example, designed optical element 111 may be configured to provide homogeneous illumination to lenslets 116 and/or to provide homogeneous collimated illumination 120.

Certain embodiments, illustrated schematically in FIGS. 7B-7F may comprise internally reflecting cavity 110 between reflector 111A and lenslet 116 and/or optical elements 116 comprising lenslets 116A within transparent layer 116B, as illustrated schematically in FIG. 7B, and/or internally reflecting cavity 110 between reflector 111A and reflective perforated elements 110D-G, illustrated schematically in FIGS. 7C-7F, respectively.

In certain embodiments, optical elements 116 may be compound and comprise multiple sub elements 116A, e.g., micro-lenses produced in different sizes and orientations, possibly with transparent layer 116B to provide collimation requirements.

In certain embodiments, illumination units 122 may comprise reflective perforated elements 110D-G limiting internally reflecting cavities 110 and having perforations 114, with element design and perforations design configured to deliver radiation to optical elements 116 in a way which optimizes collimated illumination 120.

Non-limiting examples include FIG. 7C illustrating reflective perforated elements 110D having variable density of perforations 114, having sparse perforations in a central region 114A of reflective perforated element 110D and denser perforations in peripheral regions 114B of reflective perforated element 110D. Perforation density may vary continuously and be coordinated with the design of optical elements 116 and their sub elements.

Non-limiting examples include FIG. 7D illustrating reflective perforated elements 110E having variable density and size of perforations 114, having dense and narrow perforations in a central region 114C of reflective perforated element 110E and sparser and broader perforations in peripheral regions 114D of reflective perforated element 110E. Perforation size and density may vary continuously and be coordinated with the design of optical elements 116 and their sub elements.

Non-limiting examples include FIGS. 7E and 7F illustrating reflective perforated elements 110F having variable density of perforations 114 (e.g., in regions 114E, 114F), and designed reflective sub-elements of reflective perforated elements 110F, e.g., having prism or dome shapes (illustrated schematically in FIGS. 7E and 7F respectively), possibly being reflective elements to further optimizes collimated illumination 120, e.g., make it more homogenous and/or increase the efficiency of illumination units 122. Sub-elements design and perforation size and density may vary continuously and be coordinated with the design of optical elements 116 and their sub elements.

Optionally, illumination units 122 may further comprise a reflector 111A configured to reflect radiation towards the LCD panel and possibly support lens 111 mechanically.

In certain embodiments, optical elements 116 may be designed using the Fresnel lens design principle, to yield flat lenslets with facets that provide good collimation of radiation from illumination sources 80 (e.g., in FIGS. 3A-3C) and/or openings 114 (e.g., in FIGS. 4-7F).

In certain embodiments, sub elements 116A (which are embedded within transparent layer 116B to form optical elements 116) may be designed to have different sizes and different distances from respective pinholes 114.

In any of the disclosed embodiments, diffractive optics may be used to direct collimated illumination 120 directly at specified regions of interest (ROIs) to increase efficiency and brightness substantially.

In certain embodiments, the opening area of pinpoint openings 114 may be below a specified size, such as 1 m² or much higher, such as 10 mm² per opening 114.

In certain embodiments, pinpoint openings 114 may be controlled by shutters (not shown) which are configured to control the extent of illumination from each and/or a group of pinpoint openings 114, e.g., to implement local dimming for supporting high dynamic range (HDR) displays. Control of pinpoint openings 114 may be carried out locally, e.g., by mechanical, electrical and/or optical shutters; and/or, as illumination 120 is collimated, may be carried out remotely from pinpoint openings 114 e.g., by modulating the operation of LC layer 210 according to required attenuation parameters.

In certain embodiments, the sizes of optical elements 116 (e.g., lenslets) may be e.g., 100μ²-50 cm² per element 116 such as lenslet.

In certain embodiments, optical elements 116 may comprise diffractive optics configured to collimate radiation exiting cavities 110 through pinpoint openings 114. In certain embodiments, pinpoint openings 114 may be set off the focal points of optical elements 116.

In certain embodiments, the volumes of internally reflective cavities 110 may be e.g., 1 mm³-200 cm³, per single cavity 110 illumination units 122 (see e.g., FIG. 4). Internally reflective cavities 110 may have much larger volumes when having multiple pinpoint openings 114 and/or associated with multiple optical elements 116 (see e.g., FIGS. 6A, 6B) (100μ²-50 cm²).

In certain embodiments, any of illumination source 80 may comprise one or more blue LEDs.

In certain embodiments, any of internally reflective cavities 110, pinpoint openings 114, optical elements 116, illumination sources 80 and light source layer 400 may be produced by lithographic methods, micromechanical processing, and/or any process applicable to ICs (integrated circuits) and/or MEMS (micro-electro-mechanical systems).

Certain embodiments comprise LCD 100 comprising any of disclosed backlight units 300, possibly with LCD panel 200 thereof comprising, sequentially, LC module 210, and, separate or integrated, color conversion layer 130 and color filters layer 220 (see FIGS. 1C and 2M-2S).

FIGS. 8A-E schematically illustrates white point adjustment 145 that extends a display lifetime of display 100, according to some embodiments of the invention. Illustration 145A (FIG. 8A) shows an example for EC-154 (Z₃ with JK-71+Z₂ with ES-61, see line 9 in Table 1 of U.S. Pat. No. 9,868,859, incorporated herein by reference) sample color gamut compared to DCI (digital cinema initiatives) P3 cinema standard color gamut over the CIE 1931 color space with a white region indicated by WR and a white point denoted by WP, having a diameter which is denoted by d and may be e.g., 0.01 in the diagram's x coordinates. The region WP denotes the range within which display 100 is considered to be within the specifications with respect to its color performance. Once the actual white point of display 100 is outside region WP, even when it remains within a possibly larger region WR corresponding to white color, display 100 is considered over its lifetime and not operating according to specifications. In a typical setting, films 130 are configured to provide a white point 141A at the center of the region WP and as with time RBF compounds 115 or other color conversion elements degrade 141 (indicated in graph 145C, FIG. 8C, showing the emission spectrum of film 130 by arrows which are denoted Time) white point 141A moves until it exits region WP and the display is considered over its lifetime. The degradation in terms of the distance on color diagram 145A is illustrated in graph 145B (FIG. 8B) using non-limiting experimental data of the distance from point 141A over the operation time (in arbitrary units, a.u., scaled to 1000) of the display. In some embodiments of display 100 however, film(s) 130 may be fine-tuned to have the exact white point within region WP but at a point 141B on the edge of it which is opposite to the direction of degradation marked by arrow 141 (illustrations 145D, 145E in FIGS. 8D and 8E, respectively, show an enlarged view of white region WR). Such fine tuning to white point 141A enables the display characteristics to be changed to ca. double as much as with white point 141A while staying within the specified region WP, and as a result ca. double the lifetime of display 100. The semi-quantitative example in graph 145B illustrates an increase in display lifetime, from ca. 600 a.u. to ca. 900 a.u., when changing the white-point from 141A to 141B. As a result of the change, instead of display starting exactly white and becoming somewhat colder white (see graph 145C, the green and red components decrease with time and correspondingly the blue component increases), display 100 starts a bit warmer, goes through the exact white point and ends a bit colder, with a longer lifetime overall. Setting a higher concentration of RBF compounds 115 or other color conversion elements in film 130 thus enables effective lengthening of the lifetime of display 100. Examples for increased dye concentrations may be up to 20% for green dyes and up to 40% for red dyes. Some embodiments comprising raising the concentration of one or more types of dyes (such as red-fluorescent and green-fluorescent RBF compounds 115), to fine tune the exact white point of display 100. The increased concentration of dyes may result in a somewhat warmer white within specified region WP. Illustrations 145D and 145E (FIGS. 8D, 8E) emphasize that white point 141B may be selected according to known degradation 141 of color conversion film 130 with respect to specified white point WP, for any type of film 130, including films using organic (non-rhodamine-based) or inorganic fluorescent compounds, quantum dots etc.

### Polarization

Film 130 may comprise at least one layer 134 with red fluorescent RBF compound, or at least one layer 134 with red fluorescent RBF compound and thereupon at least one layer 132 with green fluorescent RBF compound. At least one of the layers of film 130 may be configured to exhibit polarization properties.

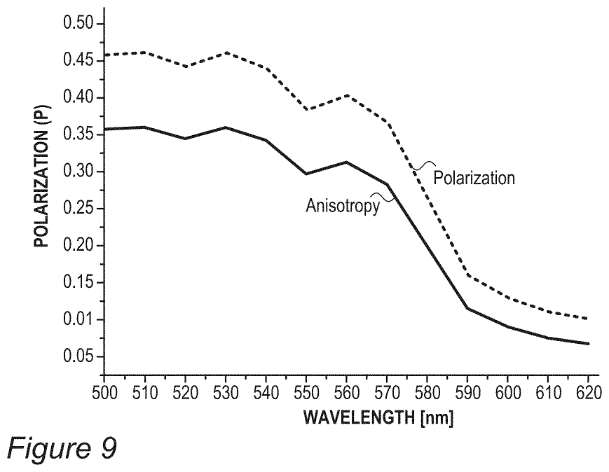

FIG. 9 is an illustration example of polarization anisotropy of film(s) 130 with RBF compound(s) 115, according to some embodiments of the invention. The inventors have found out that in certain cases, during the embedding of RBF compound(s) 115 in film 130, the molecules self-assemble to affect light polarization, providing at least partially polarized light emission. Process parameters may be adjusted to enhance the degree of polarization of light emitted from film 130, e.g., by providing conditions that cause self-assembly to occur to a larger extent. Without being bound by theory, the inventors suggest that the polarized emission of fluorescence is related to the limitations on rotational motions of the macromolecular fluorophores during the lifetime of the excitation state (limitations relating to their size, shape, degree of aggregation and binding, and local environment parameters such as solvent, local viscosity and phase transition). The inventors have further found out that these limitations may be at least partially controlled by the preparation process of film 130 which may thus be used to enhance illumination polarization in display 100.

For example, FIG. 9 illustrates polarization and anisotropy measurement of films 130 prepared with red and green fluorescent compounds (specifically, green coumarin 6 dye and rhodamine 101 red molecular dyes, using the sol gel process). In the example, the anisotropy values range between 0.3-0.5 at the emission wavelengths.

Films 130 having different red and/or green fluorescent RBF compound 115, as well as films 130 prepared by UV curing also present polarization properties and may be used in device 100 to enhance or at least partially replace polarizer films (e.g., 302, 202, 256 etc. see FIGS. 2A and 2B).

Some embodiments comprise any type of color conversion film 130, which may comprise color conversion elements other than RBF compounds 115, such as organic (non-rhodamine-based) or inorganic fluorescent compounds, quantum dots etc.—configured to provide polarize fluorescent radiation as disclosed above. Such films 130 may be used to enhance or at least partially replace polarizer films in respective displays 100.

### Red Enhancement

FIG. 10A is a high level schematic illustration of red (R) enhancement in devices with white illumination, according to some embodiments of the invention. FIG. 10A schematically illustrates a typical white light spectrum 80B-1 (of white illumination source 80B), optimized to provide RGB illumination 120 in prior art backlight units, and typical ranges (85R, 85G, 85B) of RGB filters 220 in LCD panel 200 (see FIGS. 2B, 2C and 2D). The inventors have noticed that while white light spectrum 80B-1 is optimized with respect to the ratio between its blue section (80B-B) and its yellow section (80B-Y), it is deficient with respect to the relative position of the yellow region (80B-Y) and G and R ranges 85G, 85R, respectively (corresponding, for example, to B, G, R denoted in FIGS. 2C and 2D). Indeed, much of the illumination energy in yellow region 80B-Y is filtered out and thus wasted in the operation of the display and moreover, color cross talk (part of the yellow orange might go to the green filter and some of the green-yellow to the red filter) which degrades the color gamut. The inventors have further found out that using film(s) 130 with red-fluorescent RBF compound(s) 115 (layer(s) 134) shifts 132A at least some of the illumination energy in yellow region 80B-Y into red region 85R which is passed by the R (red) filter in LCD panel 200 and is therefore not wasted. Using film(s) 130 thus increases the energy efficiency of display 100 and possibly improves its color gamut.

As illustrated in U.S. Provisional Application No. 62/488,767, incorporated herein by reference in its entirety, RGB spectrum 120 improvements may be provided by backlight unit 300 using film(s) 130. In the specific non-limiting example, films 130 were produced by UV curing process 700. White light spectrum 80B-1 is somewhat different from the one illustrated in FIG. 10A due to the difference in white light source 80B, yet also exhibits a peak in the yellow region. In contrast, emission spectrum 134-1 of film 130 (made of layer(s) 134—specifically—one to three layers with JK32 (0.02-0.3 mg/ml for each layer, spectra shown without LCD color filter effects) in backlight unit 300 splits the yellow peak of white light spectrum 80B-1 into a green and a red peak, each within the range of the corresponding G and R filters, thereby increasing the efficiency, reducing the color cross talk and improving the gamut of display 100, e.g., by providing a more saturated (narrower FWHM, full width at half maximum) red and at longer red wavelength. In the example, the characteristics of the green and red peaks of emission spectrum 134-1 of film 130 were 618±5 nm peak with FWHM of ca. 60 nm for the red peak and 518±5 nm peak with FWHM of ca. 50 nm for the green peak; with the quantum yield of film 130 being between 70-90% and the lifetime at device level being between 20,000-50,000 hours for multiple repeats.

Some embodiments comprise any type of color conversion film 130, which may comprise color conversion elements other than RBF compounds 115, such as organic (non-rhodamine-based) or inorganic fluorescent compounds, quantum dots etc.—configured to provide polarize fluorescent radiation as disclosed above. Such films 130 may be used for RGB spectra 120 by providing shifts 132A of yellow illumination 80B-Y into the red region of corresponding R color filters 220 in respective displays 100.

### Green Enhancement

In some embodiments, films 130 may be configured to provide green enhancement, using only or mostly green-fluorescent compounds.

FIG. 10B is a high level schematic illustration of green (G) and red (R) enhancement in devices with white illumination, according to some embodiments of the invention. FIG. 10B schematically illustrates a typical white light spectrum 80B-1 (of white illumination source 80B), optimized to provide RGB illumination 120 in prior art backlight units, and typical ranges (86R, 86G, 86B) of RGB filters 220 in LCD panel 210 (see FIGS. 2B-2D). In addition to red enhancement illustrated and disclosed in FIGS. 5A and 5B, the inventors have further found that further enhancement may be achieved by shifting at least some of a cyan component 80B-C in white illumination 80B into the green region (and possibly at partly further into the red region), as typically much of the illumination energy in cyan region 80B-C is filtered out by RGB filters 220 and thus wasted in the operation of the display and moreover, color cross talk (part of the greenish cyan might go to the green filter and some of the bluish cyan to the blue filter) degrades the color gamut. The inventors have further found that using film(s) 130 with green-fluorescent RBF compound(s) 115 (layer(s) 132) shifts 132B at least some of the illumination energy in cyan region 80B-C into green region 86G which is passed by G (green) filter 220 in LCD panel 210 and is therefore not wasted. Using film(s) 130 thus increases the energy efficiency of display 100 and possibly improves its color gamut.

Certain embodiments comprise LCD 100 comprising backlight unit 300 configured to provide white illumination 80B and LCD panel 210 receiving illumination (radiation) from backlight unit 300 and comprising, sequentially with respect to the received illumination: polarizing film 202, 258, liquid crystal layer 261, analyzer film 262, color conversion film 130 (possibly patterned), RGB color filter layer 220, and protective layer 266, possibly with additional analyzer film 256 between RGB color filter layer 220 and protective layer 266. Color conversion film 130 may comprise rhodamine-based fluorescent (RBF) compounds 115 selected to absorb illumination from backlight unit 300 and have an R emission peak and a G emission peak. In any of the embodiments, assistant dyes 117 may be further integrated in the color conversion film 130 and/or in a separate layer. Green enhancement in white LED applications may improve the efficiency and/or intensity of green and/or red filters 220.

### Assistant Dyes and Spectrum Shaping

FIGS. 10C-10E are high level schematic illustrations of spectrum shaping using assistant dyes 117, according to some embodiments of the invention. One or more assistant dye(s) 117 may be used, independently and/or integrated in color conversion layer(s) 130 (and/or 132, 133, 134) and/or integrated in RGB color filters 220 and/or integrated in integrated RGB color filters 220 (having color conversion compounds 115). Assistant dyes 117 are characterized herein by their absorption curve 178 and their emission (e.g., fluorescence, possibly phosphorescence) curve 179, which are shown in FIGS. 10C-10E in a schematic, non-limiting manner as triangles. Clearly realistic curves may be used to optimize displays 100 according to the disclosed principles. It is further noted that absorption and emission curves are used herein interchangeably with the terms absorption and emission peaks, respectively, in a non-limiting manner, to refer to complementary spectral characteristics of disclosed dyes 115 and/or 117. Examples for assistant dyes 117 are provided above, by Formulas (A1), (A1a), (A2) and (A3) and by compounds A1-, A1-2, A1-3, A2-1, A2-2 and A3-1.

Certain embodiments comprise shaping spectral distribution of illumination 80 delivered to RGB filters 220 using fluorescent compound(s) having one or more absorption peaks outside a respective transmission region of one of RGB filters 220 and one or more fluorescence peaks, at least one of which being inside the respective transmission region of the RGB filter. FIG. 10C illustrates an example for the R color filter, providing certain embodiments with one assistant dye 117 having an absorption curve 178 outside the transmitted range of the R filter and an intermediate emission curve 179 which partly overlaps absorption curve 178 of RBF compound 115 (in the illustrated case, red-fluorescent RBF compound 115R) to enhance the illumination absorbed thereby. In certain embodiments, multiple assistant dyes 117 may be used, having a series of absorption and emission curves (each emission curve 179 at least partly overlapping absorption curve 178 of next assistant dye 117 in the series), which form a photon delivery chain from filtered to unfiltered regions of the spectrum.

Certain embodiments comprise LCD 100 comprising backlight unit 300 configured to provide illumination 120 and LCD panel 210 receiving illumination 120 from backlight unit 300 and comprising, sequentially with respect to the received illumination: polarizing film 202, 258 liquid crystal layer 261, analyzer film 262, color conversion film 130 (possibly patterned), RGB color filter layer 220, and protective layer 266, possibly with additional analyzer film 256 between RGB color filter layer 220 and protective layer 266. Color conversion film 130 may comprise a plurality of fluorescent compounds 115, 117 selected to have, when embedded in color conversion film 130, a series of absorption peaks (or curves) 178 outside a respective transmission region of one of RGB filters 220 and respective series of fluorescence (or phosphorescence) peaks (or curves) 179, one of fluorescence peaks 179 being inside the respective transmission region of RGB filter 220 (e.g., fluorescence peak of RBF compound 115) and at least one other fluorescence peak being intermediate between the fluorescence peak inside the respective transmission region and the absorption peaks, forming a photon delivery chain from filtered to unfiltered regions of the spectrum.

Certain embodiments comprise shaping a spectral distribution of illumination 120 delivered to RGB filters 220 of LCD 100 by using at least one fluorescent compound 115 in color conversion film 130, which is selected to have, when embedded in color conversion film 130, absorption peak 178 outside a respective transmission region of one of RGB filters 220 and fluorescence peak 179 inside the respective transmission region of RGB filter 220. Correspondingly, certain embodiments comprise LCD 100 comprising backlight unit 300 configured to provide illumination 120 and LCD panel 210 receiving illumination (radiation) 120 from backlight unit 300 and comprising, sequentially with respect to the received illumination: polarizing film 202, 258 liquid crystal layer 261, analyzer film 262, color conversion film 130 (possibly patterned), RGB color filter layer 220, and protective layer 266, possibly with additional analyzer film 256 between RGB color filter layer 220 and protective layer 266. Color conversion film 130 comprises at least one fluorescent compound 115 selected to have, when embedded in color conversion film 130, absorption peak 178 outside a respective transmission region of one of RGB filters 220 and fluorescence peak 179 inside the respective transmission region of RGB filter 220.

Certain embodiments comprise shaping a spectral distribution of illumination delivered to RGB filters 220 of LCD 100 by using at least one fluorescent compound 115 and/or at least one assistant dye 117 in color conversion film 130, selected to have, when embedded in color conversion film 130, absorption curve 178 and fluorescence curve 179 selected to re-shape a spectral region of illumination 120 within a respective transmission region of at least one of RGB filters 220 to decrease FWHM (full width at half maximum) of the illumination in the respective transmission region. Correspondingly, certain embodiments comprise LCD 100 comprising backlight unit 300 configured to provide illumination 120 and LCD panel 210 receiving illumination 80 from backlight unit 300 and comprising, sequentially with respect to the received illumination: polarizing film 202, 258 liquid crystal layer 261, analyzer film 262, color conversion film 130 (possibly patterned), RGB color filter layer 220, and protective layer 266, possibly with additional analyzer film 256 between RGB color filter layer 220 and protective layer 266. Color conversion film 130 comprises at least one fluorescent compound 115 and/or at least one assistant dye 117 having, when embedded in color conversion film 130, absorption curve 178 and fluorescence curve 179—selected to re-shape a spectral region of illumination 120 within a respective transmission region of at least one of RGB filters 220 to decrease FWHM of the illumination in the respective transmission region.

As illustrates e.g., in FIG. 10E, modified illumination 80-1 may comprise components 80-1(B), 80-1(G), 80-1(R) in the transmission regions of B, G, R color filters 220, respectively, which are shaped according to requirements by one or more fluorescent compound(s) 115 and/or assistant dye(s) 117, e.g., by removal of spectral sections by absorption (e.g., any of sections 178A(B), 178A(G), possibly also a section in the red section (not shown), respectively) and/or by enhancement of spectral sections by emission (e.g., any of sections 179A(B), 179A(G), 179A(R), respectively)—as disclosed above.

In certain embodiments, LCD 100 may utilize quantum dot technology, e.g., with color conversion film 130 being based on quantum dots. Similar ideas of assistant dyes and green and red enhancement may be applied to quantum-dots-based display.

In certain embodiments, LCD 100 may utilize color conversion films 130 having asymmetric emission spectrum 76. Color conversion film 130 may further comprise one or more fluorescent compound(s) 115 and/or assistant dye(s) 117 selected to reduce a level of asymmetry in an emission spectrum of color conversion film 130. For example (see WIPO Publication No. WO 2018/042437 and U.S. Publication Nos. 2018/0072892 and 2018/0039131, incorporated herein by reference in their entirety), absorption spectrum 178 of assistant dye 117 may be selected to be reversely asymmetric, to reduce the level of asymmetry with spectral regions of RGB color filter(s) 220, e.g., B color filter 220 as illustrated in the non-limiting example.

In any of the disclosed embodiments, one or more fluorescent compound(s) 115 and/or one or more assistant dye(s) 117 may be used, independently, and/or integrated in color conversion layer(s) 130 (and/or layers 132, 133, 134) and/or integrated in RGB color filters 220 and/or integrated in integrated RGB color filters 220 (having color conversion compounds 115).

In any of the disclosed embodiments, one or more fluorescent compound(s) 115 and/or one or more assistant dye(s) 117 may be further be used to adjust the white point of LCD display 100, as illustrated e.g., in FIGS. 8C-8E.

### Spectrum Enhancement and Shaping

WIPO Publication No. WO 2018/042437 and U.S. Publication Nos. 2018/0072892 and 2018/0039131, which are incorporated herein by reference in their entirety, provide examples (see e.g., FIGS. 14A-14I) for illumination and absorption spectra, such as white illumination spectrum 80, absorption spectra 178 of red-fluorescent RBF compound 115 listed above as RS285, absorption spectra 178 of green-fluorescent RBF compound 115 listed above as ES144, absorption spectra 178 and emission spectra 179 of two non-limiting examples for assistant dyes 117—5-FAM and 5-Carboxyfluorescein (respectively), for which fluorescence enhancement by assistant dyes 117 is illustrated schematically in FIG. 10D, blue illumination spectrum 80A, absorption and emission spectra 178, 179, respectively, of assistant dye 117 (e.g., 5-FAM) as well as absorption curve 178 of red-fluorescent RBF compound 115 listed above as RS285. It is noted that in case of blue illumination spectrum 80A, phosphorous compound(s) (see above) may be selected to enhance the correspondence between the resulting illumination spectrum and absorption spectra 178 of RBF and assistant dyes 115, 117 (see also FIG. 2R above for spatial adjustment of illumination to red filter 220 only).

Various embodiments comprise methods of diverting illumination from unused spectral regions into illumination that passes through color filters 220, using one or more assistant dyes 117 which absorb unused illumination and emit usable illumination (or illumination which is further absorbed and emitted in a spectral range that is transmitted through color filter 220). It is noted that assistant dyes 117 may be selected to provide required absorption and emission spectra while maintaining good integrability in color conversion film 130 and long photostability. For example, HPTS; pyranine (8-Hydroxypyrene-1,3,6-Trisulfonic Acid, Trisodium Salt), may be used as assistant dye 117, having an absorption peak at shorter wavelengths than 5-FAM (e.g., at ca. 450 nm vs. 490 nm), with a similar emission peak at 520-530 nm (depending on embedding conditions).

FIG. 10D illustrates schematically fluorescence enhancement by assistant dyes 117, according to some embodiments of the invention. Assistant dyes 117 may be configured and used to transfer radiation from the green region of the spectrum to the red region of the spectrum by absorbing emitted green radiation and emitting the absorbed radiation in the absorption region of the red-fluorescent dye, the transfer is illustrated schematically in FIG. 10D by arrow 117C from overlap region 117A through overlap region 117B to the red emission region.

For example, relating as a non-limiting example to 5-FAM and 5-Carboxyfluorescein, the inventors estimate their effective quantum yield at ca. 90%, with high absorption coefficients of ca. 100,000/mol/L/gr. Taking RS285 and ES144 as non-limiting examples for green-fluorescent and red-fluorescent RBF compounds 115(G), 115(R), respectively, the inventors estimate an overlap area 117A (illustrated schematically as a broken-line triangle in FIG. 141) between the emission of green dye (for example RS285) 115(G) with absorbance 178 of 5-FAM 117 as being around 10-30%; and overlap area 117B (illustrated schematically as a broken-line triangle in FIG. 141) between emission 179 of 5-FAM 117 and absorbance 178 (115R) of red dye (for example ES144) 115(R) over 80%. Using these estimations, the extent of radiation 117C (illustrated schematically as an arrow from green to red) transferred from the green region to the red region of the spectrum is at least 10-30% (of the 5-FAM absorbance) times 90% (of the 5-FAM quantum yield) times 80% (of the overlap between 5-FAM emission and red dye absorption)—resulting between 7 and 20%. Moreover, the FWHM of the green fluorescence 115(G) (e.g., by RS285) becomes narrower by estimated 5-20 nm. The inventors estimate that the intensity of the red fluorescence 115(R) (e.g., by ES144) may be increased by 10-30% compared to not using assisting dyes 117. Hence, advantageously, assisting dyes 117 improve the device performance with respect to the color gamut, efficiency and/or intensity.

It is noted that 5-FAM and 5-Carboxyfluorescein may be used as assistant dyes 117 in the green region, and compounds such as red rhodamines (e.g., rhodamine 12, rhodamine 101 from Atto-Tec®, perylene dye F300 from Lumogen® etc.) may be used as assistant dyes 117 in the red region.

### Rhodamine-Based Fluorescent Molecules

A wide range of fluorescent organic molecules may be incorporated in films 130, such as materials of the xanthene dye family like fluorescein, rhodamine derivatives and coumarin family dyes, as well as various inorganic fluorescent materials. In the following, explicit examples of rhodamine-based derivatives, RBF compounds 115, are presented in detail, in a non-limiting manner.

### Red-Fluorescent RBF Compounds

Some embodiments of red-fluorescent RBF compounds 115 are defined by Formula 1.

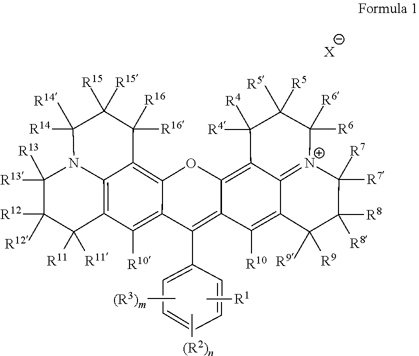

wherein
R¹ is COOR, NO₂, COR, COSR, CO(N-heterocycle), CON(R)₂, or CN;
R² each is independently selected from H, halide, N(R)₂, COR, CN, CON(R)₂, CO(N-heterocycle), NCO, NCS, OR, SR, SO₃H, SO₃M and COOR;
R³ each is independently selected from H, halide, N(R)₂, COR, CN, CON(R)₂, CO(N-heterocycle), NCO, NCS, OR, SR, SO₃H, SO₃M and COOR;
R⁴-R¹⁶ and R^{4′}-R^{16′} are each independently selected from H, CF3, alkyl, haloalkyl, cycloalkyl, heterocycloalkyl, alkenyl, alkynyl, aryl, benzyl, halide, NO₂, OR, N(R)₂, COR, CN, CON(R)₂, CO(N-Heterocycle) and COOR;
R is H, alkyl, cycloalkyl, heterocycloalkyl, alkenyl, alkynyl, aryl, benzyl, —(CH₂CH₂O)ᵣCH₂CH₂OH, —(CH₂)ₚOC(O)NH(CH₂)_{q}Si(Oalkyl)₃, —(CH₂)ₚOC(O)CH═CH₂ or —(CH₂)ₚSi(Oalkyl)₃;
n and m are each independently an integer between 1-4;
p and q are each independently an integer between 1-6;
r is an integer between 0-10;
M is a monovalent cation; and
X is an anion.

Alternatively, or complementarily, some embodiments of red-fluorescent RBF compounds 115 are defined by Formula 1, wherein

R¹ is halide, alkyl, haloalkyl, COOR, NO₂, COR, COSR, CON(R)₂, CO(N-heterocycle) or CN;
R² each is independently selected from H, halide, N(R)₂, COR, CN, CON(R)₂, CO(N-heterocycle), NCO, NCS, OR, SR, SO₃H, SO₃M and COOZ;
R³ each is independently selected from H, halide, N(R)₂, COR, CN, CON(R)₂, CO(N-heterocycle), NCO, NCS, OR, SR, SO₃H, SO₃M and COOZ;
R⁴-R⁷, R¹³-R¹⁶, R^{4′}-R^{7′} and R^{13′}-R^{16′} are each independently selected from H, alkyl, alkenyl, alkynyl, epoxide, alkylated epoxide, azide, cycloalkyl, heterocycloalkyl, aryl, benzyl, halide, NO₂, SR, OR, N(R)₂, COR, CN, CON(R)₂, CO(N-heterocycle) and COOR;
R⁸-R⁹, R¹¹-R¹², R^{8′}-R^{9′} and R^{11′}-R^{12′} are each independently selected from absent, H, alkyl, alkenyl, alkynyl, epoxide, alkylated epoxide, azide, cycloalkyl, heterocycloalkyl, aryl, benzyl, halide, NO₂, SR, OR, N(R)₂, COR, CN, CON(R)₂, CO(N-heterocycle) and COOR;
R¹⁰ and R^{10′} are each independently selected from H, alkyl, alkenyl, alkynyl, epoxide, alkylated epoxide, alkylated azide, azide, SO₃H, SO₃M, cycloalkyl, heterocycloalkyl, aryl, benzyl, halide, NO₂, SR, OR, N(R)₂, COR, CN, CON(R)₂, CO(N-heterocycle) and COOR;
R is H, haloalkyl, alkyl, cycloalkyl, heterocycloalkyl, aryl, benzyl, —(CH₂CH₂O)ᵣCH₂CH₂OH, —(CH₂)ₚOC(O)NH(CH₂)_{q}Si(Oalkyl)₃, (CH₂)ₚOC(O)NH(CH₂)_{q}Si(halide)₃, —(CH₂)ₚOC(O)CH═CH₂, —(CH₂)ₚOC(O)C(CH₃)═CH₂, —(CH₂)ₚSi(halide)₃, alkenyl, alkynyl, alkylated epoxide, alkylated azide, azide, or —(CH₂)ₚSi(Oalkyl)₃;
Z is alkyl, haloalkyl, alkenyl, alkynyl, alkylated epoxide, cycloalkyl, heterocycloalkyl, aryl, benzyl, —(CH₂CH₂O)ᵣCH₂CH₂OH, —(CH₂)ₚOC(O)NH(CH₂)_{q}Si(Oalkyl)₃, —(CH₂)ₚOC(O)CH═CH₂, —(CH₂)ₚOC(O)C(CH₃)═CH₂, or —(CH₂)ₚSi(Oalkyl)₃;
Z¹⁰¹ is O or C(CH₃)₂;
M is a monovalent cation;
n and m are each independently an integer between 1-4;
p and q are each independently an integer between 1-6;
r is an integer between 0-10;
X is an anion;
wherein if there is a double bond between the carbons which are substituted by R⁸, R^{8′}, R⁹ and R^{9′}—then R⁸ and R⁹ are absent or R⁸ and R^{9′} are absent or R^{8′} and R⁹ are absent or R^{8′} and R^{9′} are absent; and
wherein if there is a double bond between the carbons which are substituted by R¹¹, R^{11′}, R¹² and R^{12′}—then R¹¹ and R¹² are absent or R¹¹ and R^{12′} are absent or R^{11′} and R¹² are absent or R^{11′} and R^{12′} are absent.

Additional chemical species which are based on Formula 1 are provided in WIPO Publication No. WO 2018/042437 and U.S. Publication Nos. 2018/0072892 and 2018/0039131, which are incorporated herein by reference in their entirety.

The positions of R¹, (R²)ₙ and (R³)ₘ may be selected to be any feasible position with respect to the indicated ring. Any of R¹, (R²)ₙ and (R³)ₘ may be positioned at ortho, meta or para positions with respect to the rest of the molecule, as long as the resulting structure is chemically feasible. Precursors 72 and formulation 74 may be adapted to accommodate and support embodiments of the selected red-fluorescent RBF compound(s) according to the principles disclosed herein.

Specific, non-limiting, examples of red-fluorescent RBF compounds 115 which were tested below include compounds denoted ES61, JK32 (shown as JK-32A and/or JK-32B), RS56 (shown as RS56A and/or RS56B), RS106 and RS130, ES118 and ES144.

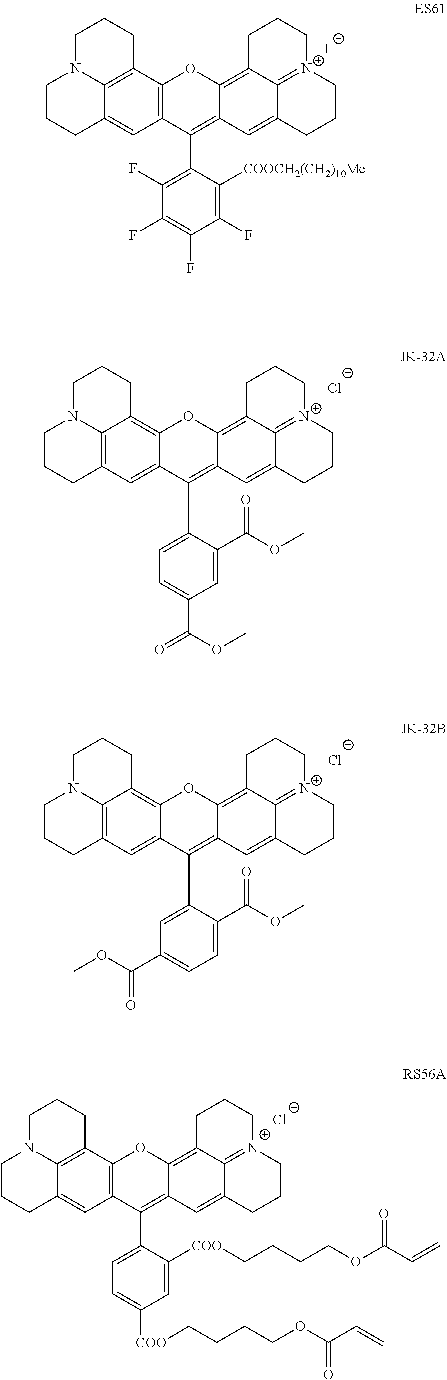

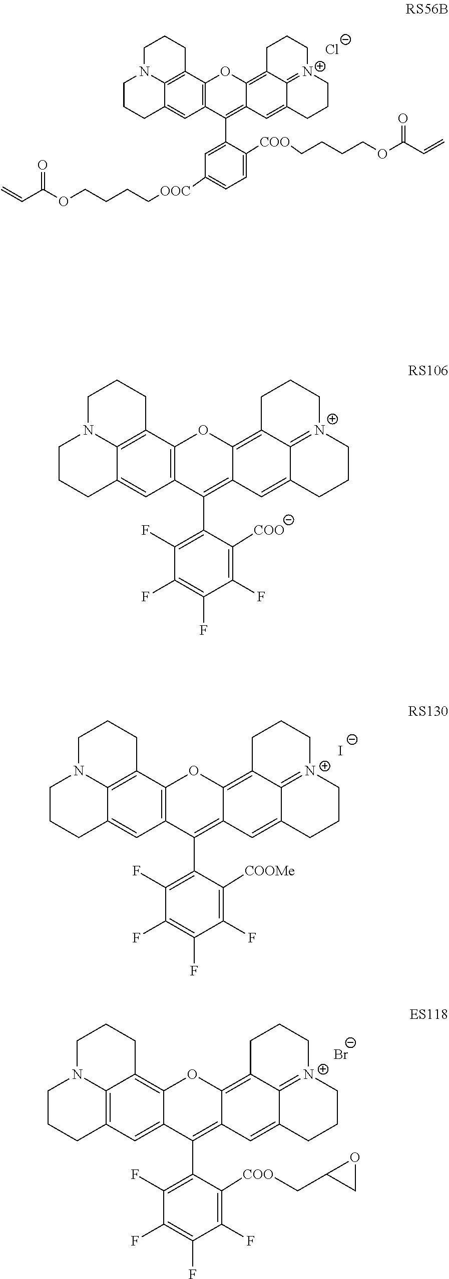

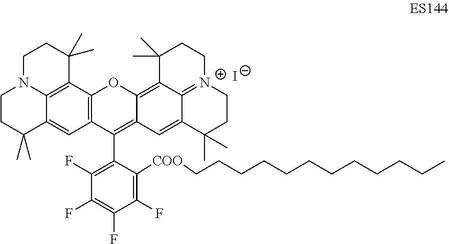

Some embodiments of red-fluorescent RBF compounds are presented in more detail in U.S. Publication Nos. 2018/0072892 and 2018/0039131, and U.S. Pat. No. 9,771,480 and are considered likewise part of the present disclosure. Non-limiting examples are provided in the following variants, numbered 1-11, 9a, 10a, 11a, 20 and 23-26.

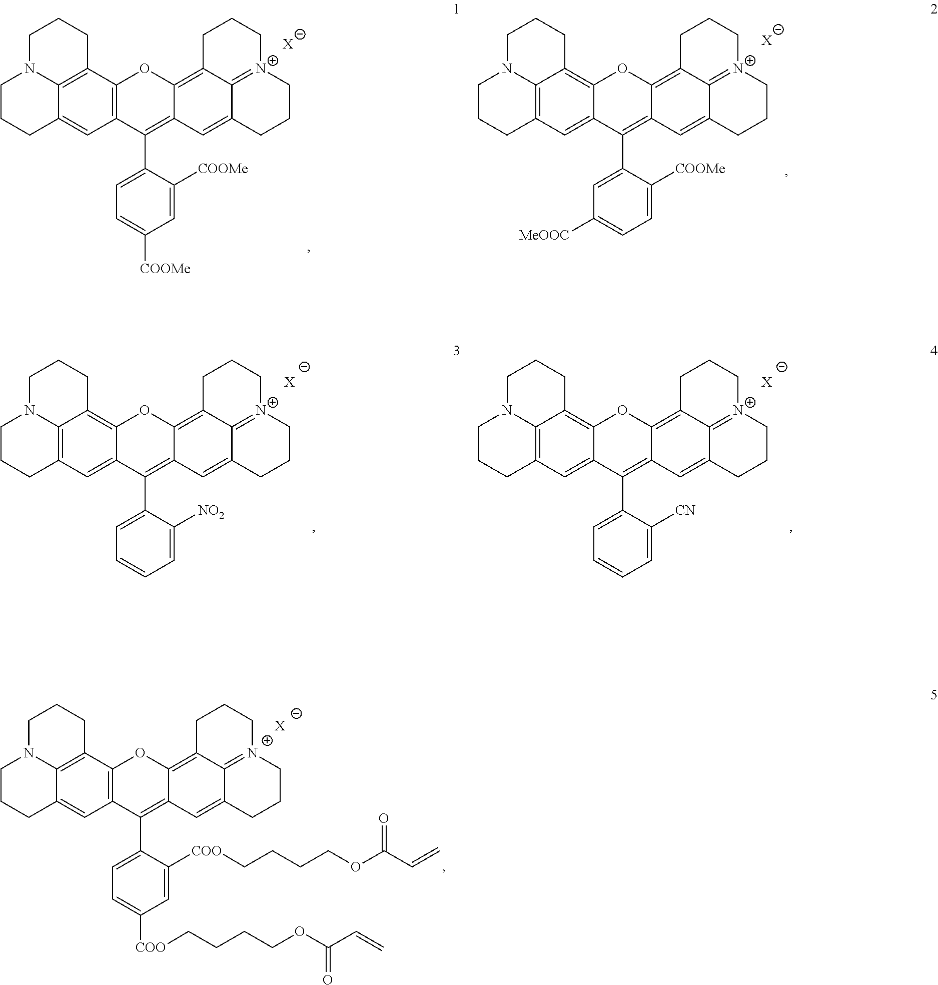

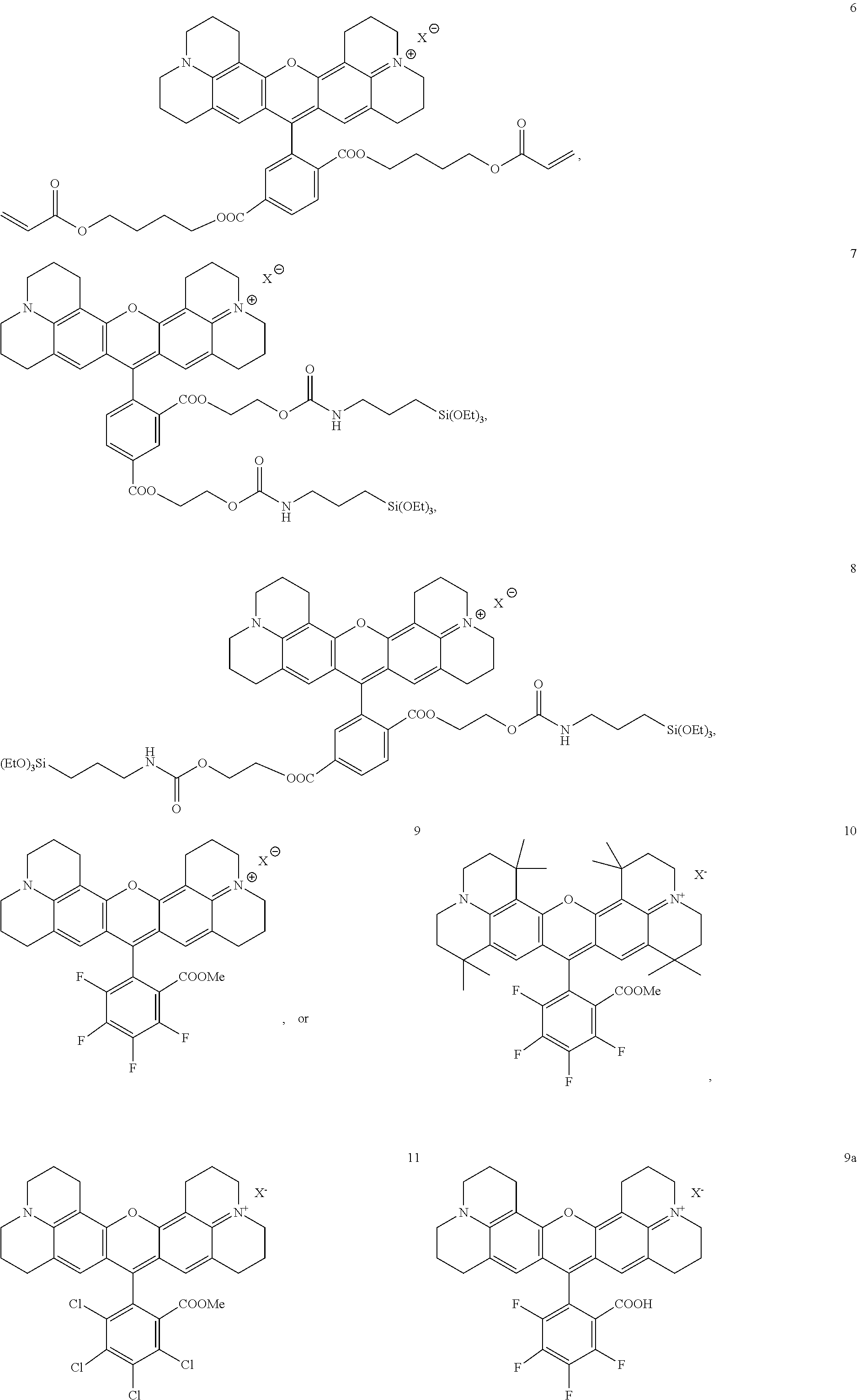

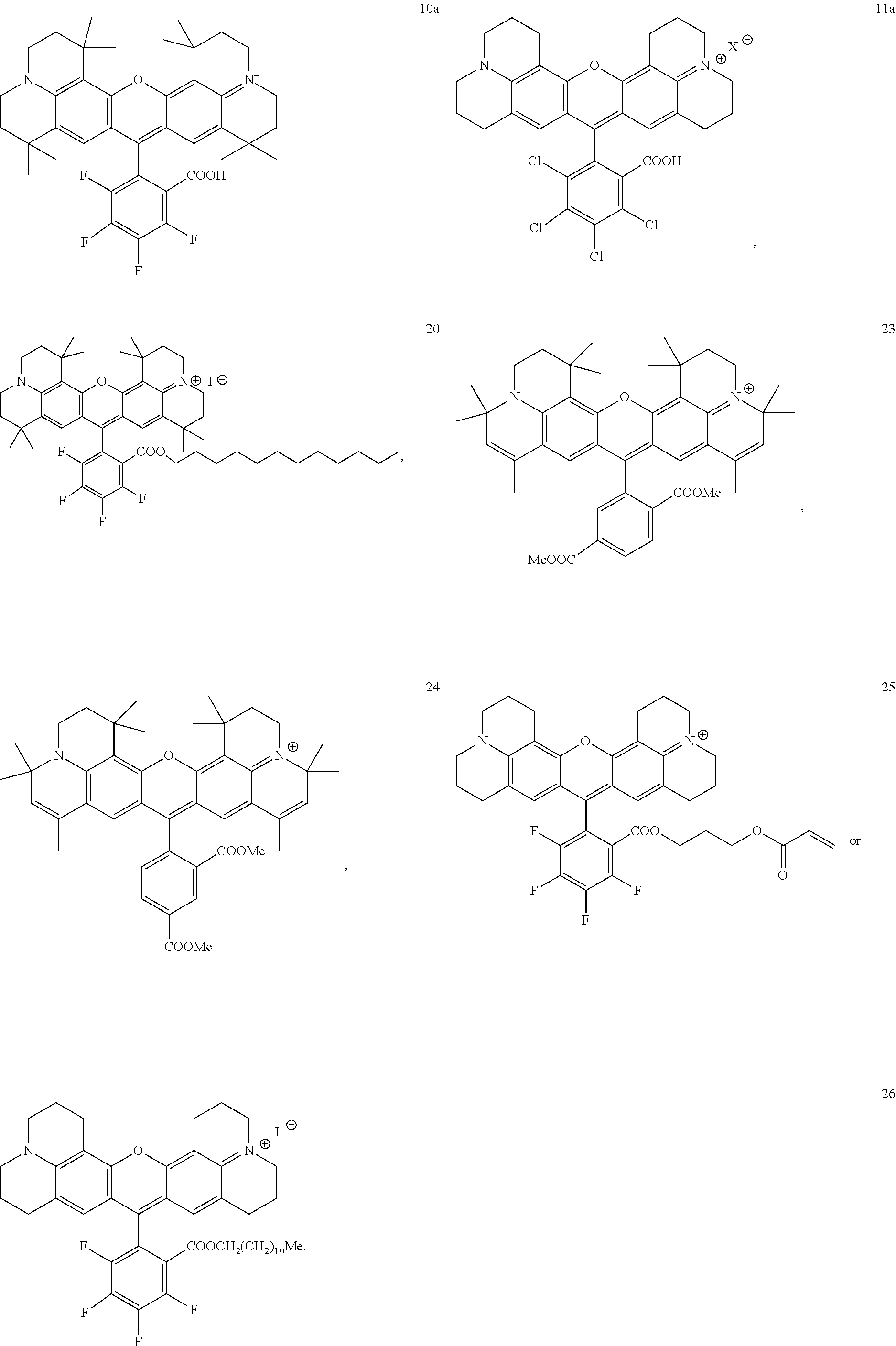

### Green-Fluorescent RBF Compounds

Some embodiments of green-fluorescent RBF compounds are defined by Formula 2.

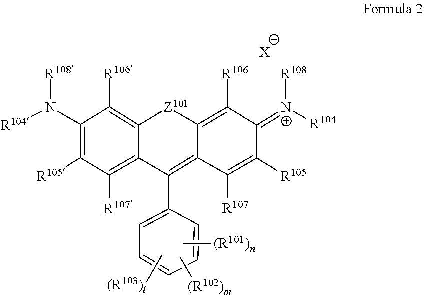

wherein
R¹⁰¹ each is independently H, Q¹⁰¹, Q¹⁰¹, C(O)Q¹⁰¹, NQ¹⁰¹Q¹⁰², NO₂, CN, SQ¹⁰¹, —NQ¹⁰¹Q¹⁰²CONQ¹⁰³Q¹⁰⁴, NCO, NCS, —OC(O)OQ¹ or halide;
R¹⁰² each is independently H, Q¹⁰¹, Q¹⁰¹, C(O)Q¹⁰¹, NQ¹⁰¹Q¹⁰², NO₂, CN, SQ¹⁰¹, —NQ¹⁰¹Q¹⁰²CONQ¹⁰³Q¹⁰⁴, NCO, NCS, —OC(O)OQ¹⁰¹ or halide;
R¹⁰³ each is independently H, Q¹⁰¹, Q¹⁰¹, C(O)Q¹⁰¹, NQ¹⁰¹Q¹⁰², NO₂, CN, SQ¹⁰¹, —NQ¹⁰¹Q¹⁰²CONQ¹⁰³Q¹⁰⁴, NCO, NCS, —OC(O)OQ¹⁰¹ or halide;
R¹⁰⁴, R^{104′}, R¹⁰⁸ and R^{108′} are each independently selected from H, alkyl, haloalkyl, heterocycloalkyl, cycloalkyl, aryl and benzyl;
R¹⁰⁵ and R^{105′} are each independently selected from H, Z′, OQ¹⁰¹, C(O)Q¹⁰¹, COOQ¹⁰¹, CON(Q¹⁰¹)₂, NQ¹⁰¹Q¹⁰², NO₂, CN, SO₃⁻, SO₃M, SO₃H, SQ¹⁰¹, —NQ¹⁰¹Q¹⁰²CONQ¹⁰³Q¹⁰⁴, NCO, NCS, alkenyl, alkynyl, epoxide, alkylated epoxide, alkylated azide, azide and halide;
R¹⁰⁶, R^{106′}, R¹⁰⁷ and R^{107′} are each independently selected from H, Q¹⁰¹, Q¹⁰¹, C(O)Q¹⁰¹, COOQ¹⁰¹, CON(Q¹⁰¹)₂, NQ¹⁰¹Q¹⁰², NO₂, CN, SO₃⁻, SO₃M, SO₃H, SQ¹⁰¹, —NQ¹⁰¹Q¹⁰²CONQ¹⁰³Q¹⁰⁴, NCO, NCS, alkenyl, alkynyl, epoxide, alkylated epoxide, alkylated azide, azide and halide;
R¹⁰⁴ and R¹⁰⁵, R^{104′} and R^{105′}, R¹⁰⁴ and R¹⁰⁸ or R^{104′} and R^{108′} may form together an N-heterocyclic ring wherein said ring is optionally substituted;
Q¹⁰¹ and Q¹⁰² are each independently selected from H, alkyl, haloalkyl, heterocycloalkyl, cycloalkyl, aryl, benzyl, —(CH₂)ₚOC(O)NH(CH₂)_{q}Si(Oalkyl)₃, —(CH₂)ₚOC(O)CH═CH₂, —(CH₂)ₚOC(O)C(CH₃)═CH₂, —(CH₂)ₚSi(Oalkyl)₃, —(CH₂)ₚOC(O)NH(CH₂)_{q}Si(halide)₃, —(CH₂)ₚSi(halide)₃, —OC(O)N(H)Q¹⁰⁴, —OC(S)N(H)Q¹⁰⁴, —N(H)C(O)N(Q¹⁰³)₂ and —N(H)C(S)N(Q¹⁰³).
Z¹⁰¹ is O or C(CH₃)₂;
Z′ is selected from alkyl, haloalkyl, heterocycloalkyl, cycloalkyl, aryl, benzyl, —(CH₂)ₚOC(O)NH(CH₂)_{q}Si(Oalkyl)₃, —(CH₂)ₚOC(O)CH═CH₂, —(CH₂)ₚOC(O)C(CH₃)═CH₂, —(CH₂)ₚSi(Oalkyl)₃, —(CH₂)ₚOC(O)NH(CH₂)_{q}Si(halide)₃, —(CH₂)ₚSi(halide)₃, —OC(O)N(H)Q¹⁰⁴, —OC(S)N(H)Q¹⁰⁴, —N(H)C(O)N(Q¹⁰³)₂ and —N(H)C(S)N(Q¹⁰³).
Q¹⁰³ and Q¹⁰⁴ are each independently selected from H, alkyl, haloalkyl, heterocycloalkyl, cycloalkyl, aryl and benzyl;
M is a monovalent cation;
n, m and l are independently an integer between 1-5;
p and q are independently an integer between 1-6; and
X is an anion.

Additional chemical species which are based on Formula 2 are provided in WIPO Publication No. WO 2018/042437 and U.S. Publication Nos. 2018/0072892 and 2018/0039131, which are incorporated herein by reference in their entirety. For example, certain embodiments having R¹⁰⁸═H are provided in these applications and are incorporated herein by reference in their entirety. Also, certain embodiments having R¹⁰⁶, R^{106′}, R¹⁰⁷, R^{107′}, R¹⁰⁸ and R^{108′} as H are provided in these applications and are incorporated herein by reference in their entirety, as well as embodiments with R¹⁰⁵ and R^{105′} being F, R¹⁰⁴ and R^{104′} being CF₃, and various examples.

Specific, non-limiting, examples of green-fluorescent RBF compounds 115 of the invention include compounds represented by the structures below, denoted as JK71 and RS285.

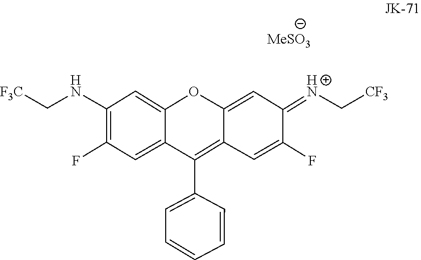

### (Z)—N-(2,7-difluoro-9-phenyl-6-((2,2,2-trifluoroethyl)amino)-3H-xanthen-3-ylidene)-2,2,2-trifluoroethan-1-aminium methanesulfonate

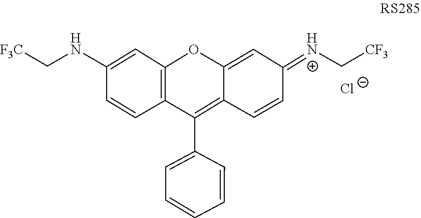

Some embodiments of green-fluorescent RBF compounds are presented in more detail in U.S. Publication Nos. 2018/0057688, 2018/0072892 and 2018/0039131 and are considered likewise part of the present disclosure. Non-limiting examples are provided in the following variants, numbered 12-19 and 21-22.

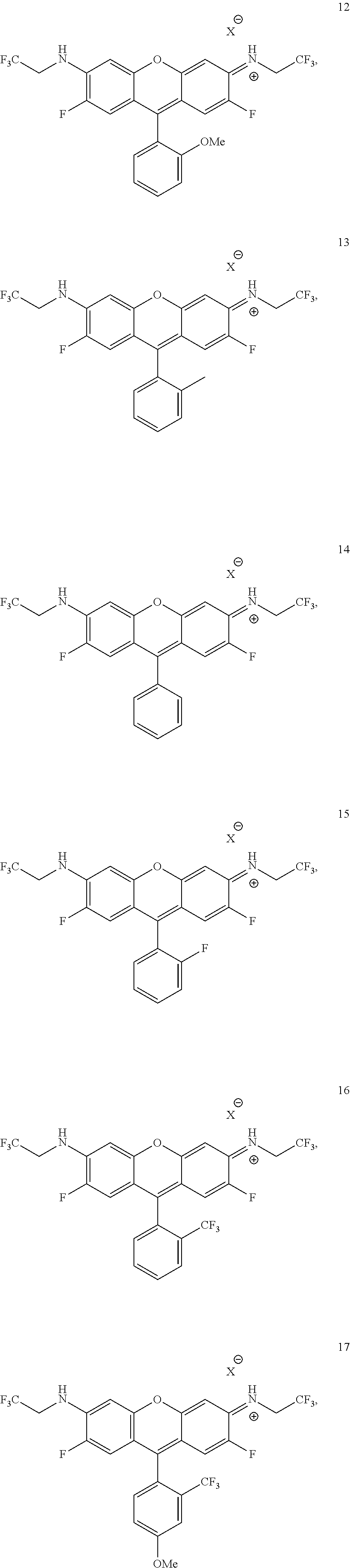

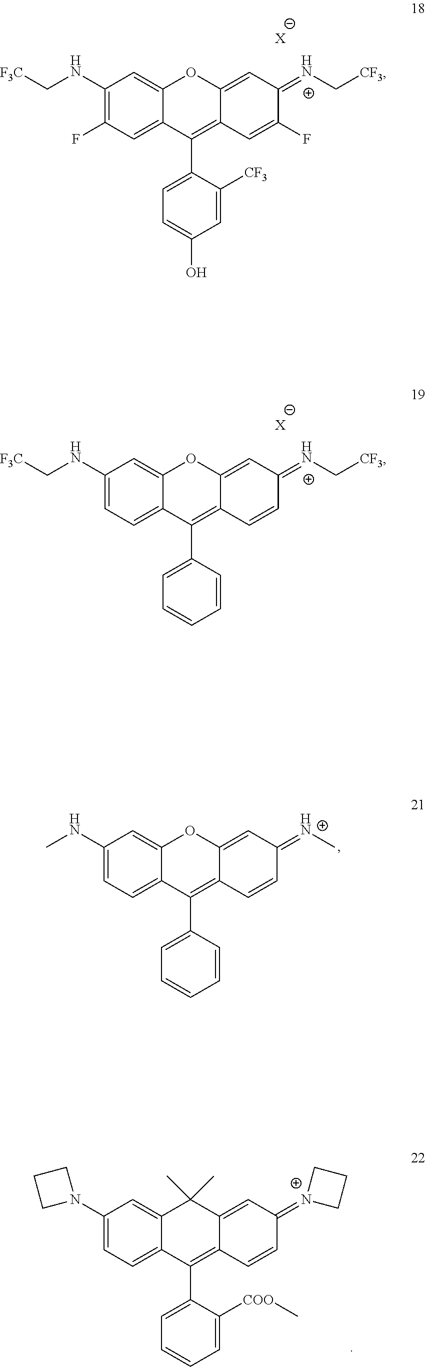

An “alkyl” group refers, in some embodiments, to a saturated aliphatic hydrocarbon, including straight-chain or branched-chain. In some embodiments, alkyl is linear or branched. In another embodiment, alkyl is optionally substituted linear or branched. In another embodiment, alkyl is methyl. In another embodiment alkyl is ethyl. In some embodiments, the alkyl group has 1-20 carbons. In another embodiment, the alkyl group has 1-8 carbons. In another embodiment, the alkyl group has 1-7 carbons. In another embodiment, the alkyl group has 1-6 carbons. In another embodiment, non-limiting examples of alkyl groups include methyl, ethyl, propyl, isobutyl, butyl, pentyl or hexyl. In another embodiment, the alkyl group has 1-4 carbons. In another embodiment, the alkyl group may be optionally substituted by one or more groups selected from halide, hydroxy, alkoxy, carboxylic acid, aldehyde, carbonyl, amido, cyano, nitro, amino, alkenyl, alkynyl, aryl. azide, epoxide, ester, acyl chloride and thiol. Each possibility represents a separate embodiment of this invention.

A “cycloalkyl” group refers, in some embodiments, to a ring structure comprising carbon atoms as ring atoms, which are saturated, substituted or unsubstituted. In another embodiment, the cycloalkyl is a 3-12 membered ring. In another embodiment, the cycloalkyl is a 6 membered ring. In another embodiment, the cycloalkyl is a 5-7 membered ring. In another embodiment, the cycloalkyl is a 3-8 membered ring. In another embodiment, the cycloalkyl group may be unsubstituted or substituted by a halogen, alkyl, haloalkyl, hydroxyl, alkoxy, carbonyl, amido, alkylamido, dialkylamido, cyano, nitro, CO₂H, amino, alkylamino, dialkylamino, carboxyl, thio and/or thioalkyl. In another embodiment, the cycloalkyl ring may be fused to another saturated or unsaturated 3-8 membered ring. In another embodiment, the cycloalkyl ring is an unsaturated ring. Non limiting examples of a cycloalkyl group comprise cyclohexyl, cyclohexenyl, cyclopropyl, cyclopropenyl, cyclopentyl, cyclopentenyl, cyclobutyl, cyclobutenyl, cycloctyl, cycloctadienyl (COD), cycloctaene (COE) etc. Each possibility represents a separate embodiment of this invention.

A “heterocycloalkyl” group refers in some embodiments, to a ring structure of a cycloalkyl as described herein comprising in addition to carbon atoms, sulfur, oxygen, nitrogen or any combination thereof, as part of the ring. In some embodiments, non-limiting examples of heterocycloalkyl include pyrrolidine, pyrrole, tetrahydrofuran, furan, thiolane, thiophene, imidazole, pyrazole, pyrazolidine, oxazolidine, oxazole, isoxazole, thiazole, isothiazole, thiazolidine, dioxolane, dithiolane, triazole, furazan, oxadiazole, thiadiazole, dithiazole, tetrazole, piperidine, oxane, thiane, pyridine, pyran, thiopyran, piperazine, morpholine, thiomorpholine, dioxane, dithiane, diazine, oxazine, thiazine, dioxine, triazine, and trioxane. Each possibility represents a separate embodiment of this invention.

As used herein, the term “aryl” refers to any aromatic ring that is directly bonded to another group and can be either substituted or unsubstituted. The aryl group can be a sole substituent, or the aryl group can be a component of a larger substituent, such as in an arylalkyl, arylamino, arylamido, etc. Exemplary aryl groups include, without limitation, phenyl, tolyl, xylyl, furanyl, naphthyl, pyridinyl, pyrimidinyl, pyridazinyl, pyrazinyl, triazinyl, thiazolyl, oxazolyl, isooxazolyl, pyrazolyl, imidazolyl, thiophene-yl, pyrrolyl, phenylmethyl, phenylethyl, phenylamino, phenylamido, etc. Substitutions include but are not limited to: F, Cl, Br, I, C₁-C₅ linear or branched alkyl, C₁-C₅ linear or branched haloalkyl, C₁-C₅ linear or branched alkoxy, C₁-C₅ linear or branched haloalkoxy, CF₃, CN, NO₂, —CH₂CN, NH₂, NH-alkyl, N(alkyl)₂, hydroxyl, —OC(O)CF₃, —OCH₂Ph, —NHCO-alkyl, COOH, —C(O)Ph, C(O)O-alkyl, C(O)H, or - or —C(O)NH₂. Each possibility represents a separate embodiment of this invention.

N-heterocycle refers to in some embodiments, to a ring structure comprising in addition to carbon atoms, a nitrogen atom, as part of the ring. In another embodiment, the N-heterocycle is a 3-12 membered ring. In another embodiment, the N-heterocycle is a 6 membered ring. In another embodiment, the N-heterocycle is a 5-7 membered ring. In another embodiment, the N-heterocycle is a 3-8 membered ring. In another embodiment, the N-heterocycle group may be unsubstituted or substituted by a halogen, alkyl, haloalkyl, hydroxyl, alkoxy, carbonyl, amido, alkylamido, dialkylamido, cyano, nitro, CO₂H, amino, alkylamino, dialkylamino, carboxyl, thio and/or thioalkyl. In another embodiment, the heterocycle ring may be fused to another saturated or unsaturated cycloalkyl or heterocyclic 3-8 membered ring. In another embodiment, the N-heterocyclic ring is a saturated ring. In another embodiment, the N-heterocyclic ring is an unsaturated ring. Non limiting examples of N-heterocycle comprise pyridine, piperidine, morpholine, piperazine, pyrrolidine, pyrrole, imidazole, pyrazole, pyrazolidine, triazole, tetrazole, piperazine, diazine, or triazine. Each possibility represents a separate embodiment of this invention.

In some embodiments, the term “halide” used herein refers to any substituent of the halogen group (group 17). In another embodiment, halide is fluoride, chloride, bromide or iodide. In another embodiment, halide is fluoride. In another embodiment, halide is chloride. In another embodiment, halide is bromide. In another embodiment, halide is iodide. Each possibility represents a separate embodiment of this invention.

In some embodiments, haloalkyl is partially halogenated alkyl. In another embodiment haloalkyl is perhalogenated alkyl (completely halogenated, no C—H bonds). In another embodiment, haloalkyl refers to alkyl, alkenyl, alkynyl or cycloalkyl substituted with one or more halide atoms. In another embodiment, haloalkyl is CH₂CF₃. In another embodiment. haloalkyl is CH₂CCl₃. In another embodiment, haloalkyl is CH₂CBr₃. In another embodiment, haloalkyl is CH₂CI₃. In another embodiment, haloalkyl is CF₂CF₃. In another embodiment, haloalkyl is CH₂CH₂CF₃. In another embodiment, haloalkyl is CH₂CF₂CF₃. In another embodiment, haloalkyl is CF₂CF₂CF₃. Each possibility represents a separate embodiment of this invention.

In some embodiments, the term “alkenyl” used herein refers to any alkyl group wherein at least one carbon-carbon double bond (C═C) is found. In another embodiment, the carbon-carbon double bond is found in one terminal of the alkenyl group. In another embodiment, the carbon-carbon double bond is found in the middle of the alkenyl group. In another embodiment, more than one carbon-carbon double bond is found in the alkenyl group. In another embodiment, three carbon-carbon double bonds are found in the alkenyl group. In another embodiment, four carbon-carbon double bonds are found in the alkenyl group. In another embodiment, five carbon-carbon double bonds are found in the alkenyl group. In another embodiment, the alkenyl group comprises a conjugated system of adjacent alternating single and double carbon-carbon bonds. In another embodiment, the alkenyl group is a cycloalkenyl, wherein “cycloalkenyl” refers to a cycloalkyl comprising at least one double bond. Each possibility represents a separate embodiment of this invention.

In some embodiments, the term “alkynyl” used herein refers to any alkyl group wherein at least one carbon-carbon triple bond (C≡C) is found. In another embodiment, the carbon-carbon triple bond is found in one terminal of the alkynyl group. In another embodiment, the carbon-carbon triple bond is found in the middle of the alkynyl group. In another embodiment, more than one carbon-carbon triple bond is found in the alkynyl group. In another embodiment, three carbon-carbon triple bonds are found in the alkynyl group. In another embodiment, four carbon-carbon triple bonds are found in the alkynyl group. In another embodiment, five carbon-carbon triple bonds are found in the alkynyl group. In another embodiment, the alkynyl group comprises a conjugated system. In another embodiment, the conjugated system is of adjacent alternating single and triple carbon-carbon bonds. In another embodiment, the conjugated system is of adjacent alternating double and triple carbon-carbon bonds. In another embodiment, the alkynyl group is a cycloalkynyl, wherein “cycloalkynyl” refers to a cycloalkyl comprising at least one triple bond. Each possibility represents a separate embodiment of this invention.

In some embodiments, the term “alkylated azide” used herein refers to any alkylated substituent comprising an azide group (—N₃). In another embodiment, the azide is in one terminal of the alkyl. In another embodiment, the alkyl is a cycloalkyl. In another embodiment, the alkyl is an alkenyl. In another embodiment, the alkyl is an alkynyl. In another embodiment, the epoxide is monoalkylated.

In some embodiments, the term “alkylated epoxide” used herein refers to any alkylated substituent comprising an epoxide group (a 3-membered ring consisting of oxygen and two carbon atoms). In another embodiment, the epoxide group is in the middle of the alkyl. In another embodiment, the epoxide group is in one terminal of the alkyl. In another embodiment, the alkyl is a cycloalkyl. In another embodiment, the alkyl is an alkenyl. In another embodiment, the alkyl is an alkynyl. In another embodiment, the epoxide is monoalkylated. In another embodiment, the epoxide is dialkylated. In another embodiment, the epoxide is trialkylated. In another embodiment, the epoxide is tetraalkylated.

In some embodiments, the notion of “ [symbol] ” of a bond within any of the disclosed structures of the current invention refers to a carbon-carbon on single bond (“ [symbol] ”) or a carbon-carbon double bond (“ [symbol] ”). In some embodiments, each structure in any of the disclosed structures of the current invention comprise two [symbol] bonds. In another embodiment, each structure comprises two [symbol] bonds that are selected to be two single bonds, two double bonds, one single and one double bond or one double and one single bond, each represents a separate embodiment of this invention.

Referring back to FIGS. 1A-1D and 2A, 2B, some embodiments comprise color conversion films 130 for LCD's 100 having RGB color filters 220 which comprise color conversion element(s) such as RBF compound(s) 115 or other compounds 76 selected to absorb illumination from backlight source 80 of LCD 100 and have a R emission peak and/or a G emission peak (see non-limiting examples below). For example, color conversion films 130 for LCD's with backlight source 80 providing blue illumination may comprise both R and G peaks provided by corresponding RBF compounds having Formula 1 and Formula 2. In another example, color conversion films 130 for LCD's with backlight source 80 providing white illumination may comprise R peak provided by corresponding RBF compound(s) having Formula 1. Color conversion film(s) 130 may be set in either or both backlight unit 300 and LCD panel 200; and may be attached to other film(s) in LCD 100 or replace other film(s) in LCD 100, e.g. being multifunctional as both color conversion films and polarizers, diffusers, etc., as demonstrated above. Color conversion film(s) 130 may be produced by various methods, such as sol gel and/or UV curing processes, may include respective dyes at the same or different layers, and may be protected by any of a protective film, a protective coating and/or protective components in the respective sol gel or UV cured matrices which may convey enhanced flexibility, mechanical strength and/or less susceptibility to humidity and cracking. Color conversion film(s) 130 may comprise various color conversion elements such as organic or inorganic fluorescent molecules, quantum dots and so forth.

### Film Production Embodiments

FIG. 11 is a high level schematic illustration of multiple film preparation steps and processes, according to some embodiments of the invention; mostly referring to sol gel processes 600, but also including UV curing processes 700, combined processes (sol gel+UV) and auxiliary processes (e.g., patterning), as well as multiple options for dye incorporation in the films (various RBF compounds 115 and their combinations, assistant dyes, protective films without dyes, etc.).

### Sol-Gel Processes

Some embodiments of fluorescent film production 70 were developed on the basis of sol gel technology in a different field of laser dyes. Reisfeld 2006 (Doped polymeric systems produced by sol-gel technology: optical properties and potential industrial applications, Polimery 2006, 51(2): 95-103) reviews sol-gel technology based on hydrolysis and subsequent polycondensation of precursors, such as organo-silicon alkoxides, leading to formation of amorphous and porous glass. The matrices for incorporation of organically active dopants are the glass/polymer composites, organically modified silicates (ORMOSIL) or hybrid materials zirconia-silica-polyurethane (ZSUR). However, the matrices taught by Reisfeld 2006 do not yield films with photo-stable fluorescent compounds that are necessary for color conversion films.

Starting from Reisfeld 2006, the inventors have found out that sol gel technology may be modified and adapted for producing films of fluorescent optical compounds which may be used in displays, with surprisingly good performance with respect to emission spectra and stability of the fluorescent compounds. The inventors have found out that multiple modifications to technologies discussed in Reisfeld 2006 enable using them in a completely different field of implementation and moreover, enable to enhance the stability of the fluorescent compounds and to tune their emission spectra (e.g., peak wavelengths and widths of peaks to enable wide color gamut illuminance from the display backlight) using process parameters. Hybrid sol-gel precursor formulations, formulations with rhodamine-based fluorescent compounds, films, displays and methods are provided, in which the fluorescent compounds are stabilized and tuned to modify display backlight illumination in a manner that increases the display's efficiency and widens its color gamut. Silane precursors are used with silica nanoparticles and zirconia to provide fluorescent films that may be applied in various ways in the backlight unit and/or in the LCD panel and improve the display's performance. The sol-gel precursor and film forming procedures may be optimized and adjusted to provide a high photostability of the fluorescent compounds and narrow emission peaks of the backlight unit.

A main, yet non-limiting, section of FIG. 11 illustrates precursors 72 and formulations 74 for sol gel films, as well as a schematic illustration of films 130 and displays 100 according to some embodiments of the invention.

WIPO Publication Nos. WO 2017/085720 and WO 2018/042437, and U.S. Publication Nos. 2018/0072892 and 2018/0039131, and U.S. Pat. No. 9,868,859 provide additional details as well as comparison to the prior art and are incorporated herein by reference in their entirety.

Hybrid sol-gel precursor formulations 72 comprise an epoxy silica ormosil solution 106 prepared from TEOS (tetraethyl orthosilicate) 102, at least one silane precursor 104 and/or MTMOS (methyltrimethoxysilane) 91B, and GLYMO 91C; a nanoparticles powder 109 prepared from isocyanate-functionalized silica nanoparticles 111, or non-functionalized silica nanoparticles 111, and ethylene glycol 108; and a transition metal(s) alkoxide matrix solution 103 (based on e.g., zirconia, titania or other transition metal(s) alkoxides). The ratios (wt/vol/vol (mg/ml/ml)) of nanoparticles powder/epoxy silica ormosil solution/transition metal(s) alkoxide matrix solution may be in the range 15-25/1-3/1, with each of the components possibly deviating by up to 50% from the stated proportions. Additional variants 107 are provided below. FIG. 11 presents non-limiting examples of process 600.

In a non-limiting example, the epoxy silica ormosil solution and the transition metal(s) alkoxide matrix solution may be mixed at ratio of between 1:1 and 3:1 (e.g., 2:1) followed by adding the nanoparticles powder at a concentration of 5-10 mg/1 ml mixed (e.g., epoxy silica ormosil solution and zirconia) solution—resulting in ratios (wt/vol/vol (mg/ml/ml)) of nanoparticles powder/epoxy silica ormosil solution/transition metal(s) alkoxide matrix solution of 15-30/2/1 in the non-limiting example, wherein any of the components may deviate by up to ±50% from the stated proportions. The solution may then be mixed (e.g., for one hour) and then filtered (e.g., using a syringe with a 1 μm filter). The fluorophore may then be added to form formulation 74 from precursor 72, and the mixing may be continued for another hour. Formulation 74 may then be evaporated and heated (e.g., in a non-limiting example, using a rotovap under pressure of 60-100 mbar and temperature of 40-60° C.) to achieve increased photo-stability as found out by the inventors and explained below.

### Epoxy Silica Ormosil Solution

Specifically, compared to the process of Reisfeld 2006, the inventors have found out that replacing TMOS by TEOS 102 and using different silane precursors 104 provide epoxy silica ormosil solution 106 which enables association of rhodamine-based fluorescent (RBF) compounds 115 in resulting films 130 which are usable in displays 100, which prior art ESOR does not enable. In particular, the inventors have used various silane precursors 104 to enhance stability of and provide emission spectrum tunability to RBF compounds 115 in produced film 130, as shown in detail below.

For example, silane precursors 104 may comprise any of: MTMOS (methyltrimethoxysilane), PhTMOS, a TMOS with fluorine substituents, e.g., F₁TMOS (trimethoxy(3,3,3-trifluoropropyl)silane), F₀TEOS (Fluorotriethoxysilane) or F₂TMOS (tridecafluoro-1,1,2,2-tetrahydrooctyl)trimethoxysilane, 1,2-bis(triethoxysilyl)ethane, trimethoxy(propyl)silane, octadecyltrimethoxysilane, fluorotriethoxysilane, and ammonium(propyl)trimethoxysilane. The first three options are illustrated below.

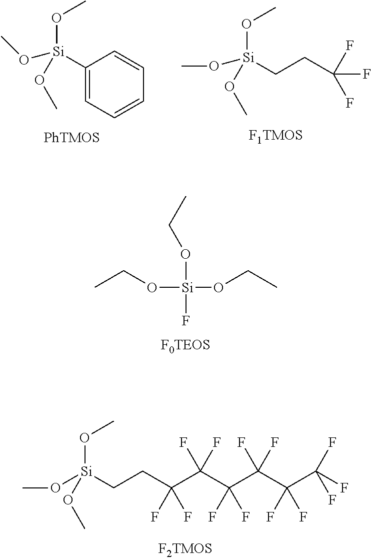

In certain embodiments, silane precursors 104 may comprise any alkoxysilane, with R¹, R², R³ typically consisting of methyl or ethyl groups (e.g., R⁴—OSi(Me)₃), and R⁴ may consist of a branched or unbranched carbon chain, possibly with any number of halogen substituents, as illustrated below.

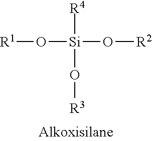

In certain embodiments, silane precursors 104 may comprise any of: tetraalkoxysilane (e.g., tetraethoxysilane), alkyltrialkoxysilane, aryltrialkoxysilane, haloalkyltrialkoxysilane, heterocycloalkyltrialkoxysilane, N-heterocycletrialkoxysilane, (3-Glycidyloxypropyl)trialkoxysilane, haloalkyltrialkoxysilane, heterocycloalkyltrialkoxysilane, N-heterocycletrialkoxysilane, and cycloalkyltrialkoxysilane.

In certain embodiments, silane precursors 104 may be selected from any of the following structures:

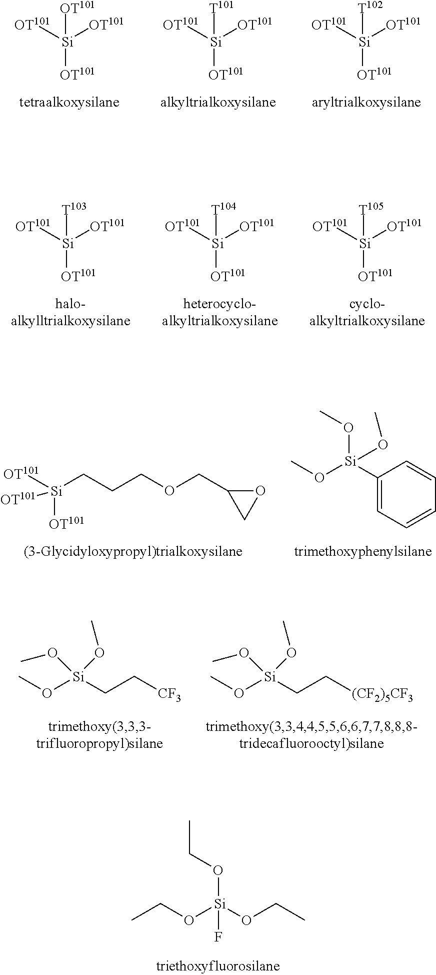

wherein T101 is an alkyl, T102 an aryl, T103 an haloalkyl, T104 an heterocycloalkyl (including a N-heterocycle) and T105 an cycloalkyl, as defined herein.

In certain embodiments, silane precursors 104 may comprise, in addition or in place of silane precursor 104 disclosed above, at least one of: 1,2-bis(triethoxysilyl)ethane, trimethoxy(propyl)silane, octadecyltrimethoxysilane, fluorotriethoxysilane, ammonium(propyl)trimethoxysilane (illustrated below) and any further varieties of any of disclosed silane precursor 104.

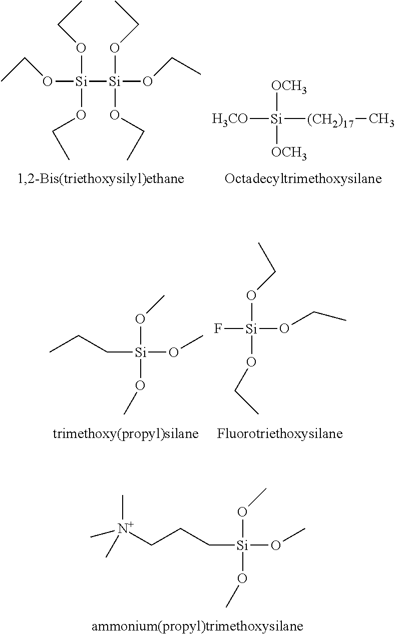

In certain embodiments, epoxy silica ormosil solution may be prepared by first mixing the TEOS and the at least one silane precursor(s) under acidic conditions and then adding the GLYMO. The acidic conditions may be adjusted by adding acetic acid, followed by adding water and alcohol(s) such as ethanol, propanol, 2-propanol or butanol.

In certain embodiments, the volumetric ratio between TEOS:MTMOS or other silane precursor(s):GLYMO may be between 1:1:1.5-2; and the volumetric ratio between TEOS:silane precursor(s):acetic acid:alcohol:water may be between 1:1:0.01-1:1-10:4-8. Epoxy silica ormosil solution mixing time may be reduced to five minutes. Any of the components may deviate by up to ±50% from the stated proportions.

In some embodiments (e.g., additional variants 107), ethanol and/or water are not used, to simplify the process. For example, diphenylsilanediol (DPSD) may be used to provide a water-free matrix, avoiding the first hydrolysis step in the condensation.

In some embodiments (e.g., additional variants 107), citric acid and/or ascorbic acid may replace or be added to the acetic acid.

### Nanoparticles Powder

Nanoparticles powder 109 is prepared from ethylene glycol 108 and isocyanate-functionalized silica nanoparticles (IC-Si NP) 111 or non-functionalized silica nanoparticles 111.

The inventors have found out that using ethylene glycol 108 for nanoparticles powder 109 instead of polyethylene glycol for DURS (diurethane siloxane) (as in Reisfeld 2006) enables better control of the film production and better films 130 than prior art sol-gel precursors, as explained below.

IC-Si NP 111 are multi-functional nanoparticles which have many active sites and specifically many more then prior art 3-isocyanatopropyltriethoxysilane (ICTEOS) 94B which is not multi-functionalized. ICTEOS has a single isocyanate group and when two ICTEOS molecules bind to PEG they create diuretane silane (DURS); while IC-Si NP has many active sites which may form significantly different matrix structures.

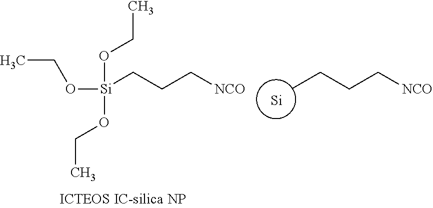

IC-Si NP have hydroxide groups on their surface which participate in the condensation step (detailed below), and accordingly increase the actual functionality of the IC-Si NP.

The inventors have found that using IC-Si NP 111 for nanoparticles powder 109 instead of prior art 3-isocyanatopropyltriethoxysilane (ICTEOS) may produce films with a tighter matrix and may limit the diffusion of the RBF compound and inhibit reactive molecules from reaching the RBF compound. The matrix may also absorb residue solvents and unreacted precursors thereby protecting RBF compound from potential reactions that may occur with the residue solvents and unreacted precursors.

The isocyanate-functionalized silica nanoparticles (IC-Si NP) may comprise (isocyanato)alkylfunctionalized silica nanoparticles and/or 3-(isocyanato)propyl-functionalized silica nanoparticles, which may be prepared from precursors (isocyanato)alkylfunctionalized trialkoxysilane and/or 3-(isocyanato)propyltrietoxysilane, respectively.

In some embodiments, nanoparticles 111 may comprise non-functionalized silica nanoparticles. The non-functionalized silica nanoparticles 111 may be comprised of any silica nanoparticles. In some embodiments, the non-functionalized silica nanoparticles 111 may comprise standard silica gel (CAS 7631-86-9).

The nanoparticles powder may be prepared by mixing and refluxing the silicon (e.g. IC-Si NP) and glycolated precursors. In some embodiments, the ethylene glycol may be added in excess. In some embodiments, the reflux may be followed by cooling and filtration steps. In some embodiments, chlorobenzene (C₆HCl) may be added to the mixture before the reflux step. In some embodiments, the chlorobenzene (C₆H₅Cl) may be evaporated prior to the cooling step. In an example, nanoparticles powder was prepared by refluxing 3-isocyanatopropyl functionalized nanoparticles and ethylene glycol. In one embodiment, about 50-150 mg of 3-isocyanatopropyl functionalized silica nanoparticles (with 200-400 mesh, 1.2 mmol/g loading) and 16-320 μl of ethylene glycol were refluxed in chlorobenzene for about 2-6 hours. The functionalized silica nanoparticles were then separated from the chlorobenzene by a rotary evaporator.

In various embodiments, the size of the silica nanoparticles may be any of between about 1-500 nm, between about 1-400 nm, between about 1-100 nm, between about 50-300 nm, between about 50-200 nm, between about 100-200 nm, between about 100-160 nm and/or between about 110-140 nm.

U.S. Provisional Patent Application Nos. 62/593,936 and 62/613,085, incorporated herein by reference in their entirety, provide a high-resolution SEM image of a sol-gel film prepared with IC-Si NP which clearly shows there are nanoparticles within the sol-gel matrix. In certain embodiments, using IC-silica NP, as opposed to ICTEOS, increases the photostablity of the film from one day with ICTOS to three days with IC-silica NP. In this example both films were prepared using JK71 as the RBF molecule in a Z3 matrix and the measurements were done by a Fluorimeter, FluoroMax-4 Horiba, the excitation was: 452 nm, the temperature was: 70° C. and the flux 70 mW/cm.

In some embodiments, the non-functionalized nanoparticles 111 may replace the functionalized nanoparticles in both Z2 and Z3 matrix using the same concentration by weight of the particles per volume of the solution.

U.S. Provisional Patent Application Nos. 62/593,936 and 62/613,085, incorporated herein by reference in their entirety, also show a photo-stability comparison between a device with functionalized silica NP and non-functionalized silica NP, with the latter, in some embodiments, providing at least the same photostability. Nanoparticles powder 109 may be prepared from a mixture of functionalized and non-functionalized silica NP. In some embodiments the ratio of functionalized and non-functionalized silica NP in the mixture may be any of 50:50, 40:60, 20:80, 10:90, 60:40, 70:30, 80:20 and 90:10. In some embodiments, the size of the functionalized NP is between about 1-400 nm and the size of the non-functionalized NP is between about 1-100 nm. In some embodiments, the size of the functionalized NP is between about 50-300 nm and the size of the non-functionalized NP is between about 50-200 nm. In some embodiments, the size of the functionalized NP is between about 100-200 nm and the size of the non-functionalized NP is between about 100-160 nm. In some embodiments, the size of the functionalized NP is between about 110-140 nm and the size of the non-functionalized NP is between about 1-400 nm. Any of the above embodiments may be combined together. In some embodiments (e.g., additional variants 107), all nanoparticles may be functionalized, or all nanoparticles may be non-functionalized.

In some embodiments (e.g., additional variants 107), nanoparticles powder is not used, to simplify the process.

### Transition Metal(s) Alkoxide Matrix Solution

Transition metalalkoxide matrix solution 103 may comprise alkoxides of one or more transition metals. For example, a zirconia (ZrO₂) matrix solution may be prepared from zirconium tetraalkoxide, e.g., Zr(OPr)₄ and/or zirconium, mixed with alcohol (e.g., propanol) under acidic conditions (e.g., in the presence of acetic acid, citric acid and/or ascorbic acid). Various transition metals alkoxides may be used in place or in addition to zirconia.

In certain embodiments, the epoxy silica ormosil solution may be mixed with the zirconia matrix solution at a 2:1 volumetric ratio, and the nanoparticles powder may then be added to the mixture to provide, after mixing (e.g., for 1-5 hours) and filtering, hybrid sol-gel precursor formulations. The zirconia matrix solution may be configured to catalyze the epoxy polymerization of the epoxy silica ormosil solution. In some embodiments, the zirconia matrix solution may be added to the epoxy silica ormosil solution after e.g., 15, 30, 45 minutes. The subsequent mixing time may be decreased down to 10 minutes.

In some embodiments, other metal oxide matrix may be used instead or in addition to zirconia matrix during the sol-gel process, such as titania using titanium isopropoxide or boron oxide using boric acid. Zirconia and/or alkoxides from transition metals such as boron alkoxide 103 may be used in preparing sol-gel precursor 72.

### Formulation

Formulations 74 comprise hybrid sol-gel precursor formulations 72 and at least one RBF compound 115 such as red-fluorescent RBF compound(s) and green-fluorescent RBF compound(s) which may be configured to emit the R and G components of the required RGB illumination, provided by the display's backlight unit (red-fluorescent RBF compounds emit radiation with an emission peak in the red region while green-fluorescent RBF compounds emit radiation with an emission peak in the green region). It is emphasized that formulations 74 are very different from prior art laser dye formulation as laser dye usage as gain medium is very different from the operation of fluorescent films in the backlight unit, e.g., concerning stability, emission spectra and additional performance requirement as well as operation conditions.

Stages of methods 600—namely preparing hybrid sol-gel precursor formulation 72 (stage 610), mixing in RBF compound(s) 115 to form formulation 74 (stage 620), forming film 130 (stage 630) and optionally evaporating alcohols prior to film formation (stage 625)—are shown schematically and explained in more detail below.

The mixture of the hybrid sol-gel precursor formulation and the RBF compound(s) may be stirred and then evaporated and heated (e.g., in a non-limiting example, stirred for between 20 minutes and three hours, evaporated at 60-100 mbar and heated to 40-60° C.) to increase the photo-stability of the RBF compound(s) (see additional process details below). Process parameters may be adjusted to avoid damage to the fluorescent dyes, control parameters of the sol gel process and optimize the productivity in the process.

The evaporation of alcohols from the sol-gel prior to the coating of the substrate may form a denser matrix which provides a tight packaging for the RBF compound. The tighter packaging may result in higher photostablity as can be seen in FIG. 7C of U.S. Publication No. 2018/0072892 incorporated herein by reference. The figure is a graph showing the normalized intensity, with and without an evaporation step, of a film of Z1 formulation (detailed below) with RS130 as the RBF molecule. As can be seen the stability of the layer with evaporation (dark color line) is almost twice that of the layer without evaporation (light color line). Details of the measurement are: Fluorimeter, FluoroMax-4 Horiba, Excitation: 540 nm; Detail of acceleration: Excitation: 452 nm; Temperature: 70° C.; Flux: 70 mW/cm².

The concentration of the RBF compound(s) may be adjusted to determine the final peak emission intensity excited by the chosen backlight unit and may range e.g., between 0.005-0.5 mg/ml. It is noted that multiple fluorescent molecules having different emission peaks may be used in a single formulation 74. The processes may be optimized to achieve required relations between the RBF compound(s) and the other components of the film, e.g., to achieve any of supramolecular encapsulation of the RBF compound(s) in the sol gel matrix, covalent embedding of the RBF compound(s) in the sol gel matrix (e.g., via siloxane bonds), and/or incorporation of the RBF compound(s) in the sol gel matrix.

Silane precursors 104 may be selected according to the used RBF compound. For example, the inventors have found out that PhTMOS may be used to stabilize red-fluorescent RBF compounds. In another example, the inventors have found out that TMOS with fluorine substituents may be used to stabilize red-fluorescent RBF compounds. Modifying and adjusting parameters of the substituents was found to enable control of the photostability and emission characteristics of the fluorescent compounds. In yet another example, the inventors have found out that F₁TMOS may be used to stabilize green-fluorescent RBF compounds. These and more findings are presented below in detail.

### Optimizing the Silane Precursors in the Epoxy Silica Ormosil Solution to Stabilize and Tune the Fluorescent Molecules

Films 130 prepared from formulation 74 may comprise epoxy silica ormosil solution 106 prepared from TEOS 102, at least one silane precursor 104 (and/or MTMOS 91B), and GLYMO 91C; nanoparticles powder 109 prepared from isocyanate-functionalized silica nanoparticles 111 or non-functionalized silica nanoparticles 111 and ethylene glycol 108; a transition metal(s) alkoxide matrix solution 103; and at least one RBF compound 115, selected to emit green and/or red light and being supramolecularly encapsulated and/or covalently embedded within film 130. Silane precursors 104 may comprise any of MTMOS, PhTMOS, a TMOS with fluorine substituents, F₁TMOS, F₂TMOS (tridecafluoro-1,1,2,2-tetrahydrooctyl)trimethoxysilane, 1,2-bis(triethoxysilyl)ethane, trimethoxy(propyl)silane, octadecyltrimethoxysilane, fluorotriethoxysilane, and ammonium(propyl)trimethoxysilane. For example, for film 130 and/or film layer 134 with red-fluorescent RBF compound, silane precursor 104 may comprise PhTMOS and/or a TMOS with fluorine substituents. In another example, for film 130 and/or film layer 132 with green-fluorescent RBF compound, silane precursor 104 may comprise F₁TMOS.

Examples are provided below for four matrix compositions (Z₁, Z₂, Z₃, Z₄) for mixtures of epoxy silica ormosil solution and zirconia matrix solution having the components Zr(PrO)₄:GLYMO:TEOS:silane precursor at n=0.011:0.022:0.013:0.021 (moles), with the silane precursor being MTMOS in Z₁, PhTMOS in Z₂, F₁TMOS in Z₃, and F₂TMOS in Z₄, as illustrated below.

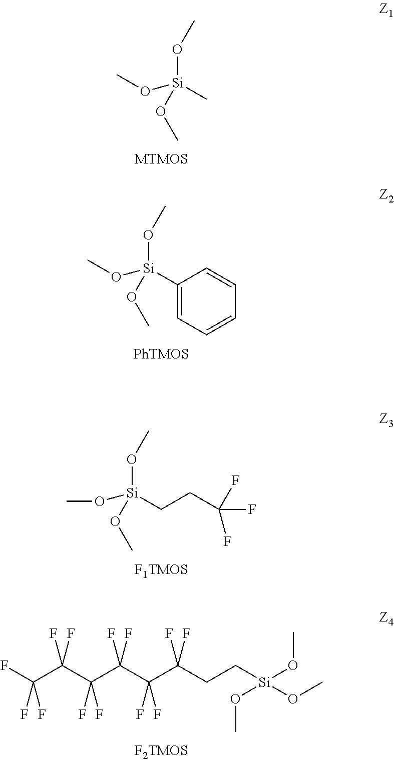

These matrices were mixed with several dyes and tested, as corresponding films 130, for quantum yield and lifetime, as presented in detail below, with results presented in WIPO Publication No. WO 2017/085720 and U.S. Pat. No. 9,868,859, which are incorporated herein by reference in their entirety, and demonstrate the capabilities of the disclosed technology to increase the lifetime of RBF compound(s) in film 130 multiple times over, reach high quantum yields, tune the emission peak wavelength of the RBF compound(s) significantly and provide tuned multi-layered films 130. Specifically, intercalating the red fluorescent compound(s) in the Z₂ matrix resulted in increased photo-stability, intercalating the green fluorescent compound(s) in the Z₃ matrix resulted in increased photo-stability and improved the QY (quantum yield) compare to the Z₁ matrix. When combining the precursor of Z₂ and Z₃ together, changing the PhTMOS:F₁TMOS ratio can provide tuning of the green wavelength.

The inventors have also found out that the length of the carbon chain of the silane precursor(s) may contribute to the stability of the red-fluorescent RBF compounds; in certain embodiments the carbon chain may consist of 8, 9, 10, 12 or more carbon atoms, possibly with corresponding fluorine atom as hydrogen substituents. In certain embodiments, some or all fluorine atoms may be replaced by another halogen such as chlorine. Moreover, the inventors have found out that modifying the length and hydrophobic\hydrophilic degree of the chain may be used to further tune and adjust the emission peak (beyond the data exemplified above), according to requirements.

The inventors have used various silane precursors 104 to provide emission spectrum tunability to film 130. In some embodiments tuning of the wavelength may be achieved by adjusting the ratio of the silane precursors 104. In some embodiments, the ratio of silane precursors is adjusted within each layer; such as a 1:1 ratio of PhTMOS and F₁TMOS in a single sol-gel matrix layer. In some embodiments, the ratio of the silane precursors is adjusted between layers; such as a 1:1 ratio of layers—for example one layer with PhTMOS and one layer with F₁TMOS one on top of each other.

U.S. Pat. No. 9,868,859 and U.S. Publication No. 2018/0072892, incorporated herein by reference in their entirety, further provide an example of a peak shift due to the change in molar ratio of two silane precursors PhTMOS (Z3 matrix detailed below): F₁TMOS (Z2 matrix detailed below). As can be seen (in FIG. 8F of U.S. Publication No. 2018/0072892) the first peak with just Z3 is at 535 nm and as Z2 is added and the ratio changes the peak shifts to higher wave lengths up to 545 nm when the ratio is 3:1. The wavelengths for each ratio can be found in rows 5-8 in Table 1 of U.S. Pat. No. 9,868,859. In this example JK71, a green RBF molecule, was used in a concentration of 0.15 mg/ml, in a single layer of ˜40 μm thickness.

In some embodiments, the GLYMO precursor is polymerized 107C (poly-GLYMO) before it is used in the epoxy silica ormosil solution preparation. See example below:

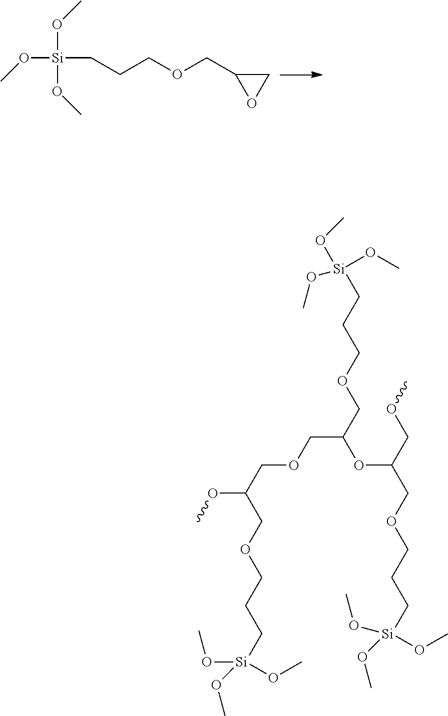

Using poly-GLYMO 107C in the preparation of the hybrid sol-gel matrix may result in an increase of the crosslinking density.

In some embodiments GLYMO is polymerized in the presence of at least one RBF compound. This may provide a polymer cage which limits the diffusion of the RBF compound and inhibits reactive molecules from reaching the RBF compound.

In some embodiments, the RBF compound has epoxide groups which enable it to covalently bind to the sol-gel's polymer back bone thus further limiting the RBF diffusion. In some embodiments, the RBF compound is ES-118 according to the following formula:

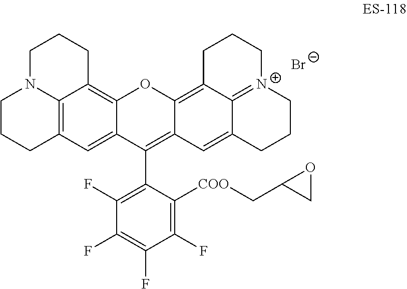

In some embodiments (3-Glycidyloxypropyl)trimethoxysilane (Glymo CAS: 2530-83-8) was dissolved in ethanol in concentration of 1-10 mM. Then to initiate the polymerization 1-methylimidazole (CAS: 616-47-7) was added, in concentration of 0.05%-5% (w/w), the solution was then maintained under reflux for three (3) hours.

In some embodiments, the poly-glymo:TEOS ratio is about 1:1-3:1 (v/v).

### Epoxy Silica Ormosil Solution Additives

There is a positive relation between the crosslinking density of a matrix and the photo-stability of the trapped fluorophore. Additives 107, described below, increase the crosslinking density of the hybrid sol-gel matrix and have additional advantages detailed below.

In some embodiments one or more additional additives 107 may be added to the epoxy silica ormosil solution. In some embodiments, the additives are added during the preparation of the epoxy silica ormosil solution and specifically following the addition of the silane precursors.

### Polydimethylsiloxane Hydroxy Terminated

In some embodiments additive 107 may be polydimethylsiloxane hydroxy terminated (PDMS-hydroxy CAS: 70131-67-8) as illustrated below. PDMS is highly flexible (has a very low Tg) and highly hydrophobic. The PDMS's hydroxyl groups on both sides of the main chain allow covalent linkage to the sol-gel matrix and act as flexible crosslinkers.

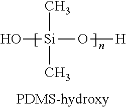

In some embodiments PDMS was added in a molecular weight of 0.1-20 (kDa) and in a concentration of 5%-20% (w/w). The resulting hybrid sol-gel had a higher viscosity, enabled more uniform spreading, increased flexibility, reduction of bubbles, better resistant to thermal shock, less splintering during cutting and better resistance toward humidity compared to the hybrid sol-gel without PDMS.

WIPO Publication No. WO 2018/042437 and U.S. Publication Nos. 2018/0072892 and 2018/0039131, incorporated herein by reference in their entirety, provides a comparison of a film with and without PDMS-hydroxyl, which demonstrates how the addition of PDMS-hydroxyl advantageously prevents the bubbling effect and produces a smoother surface.

### Dendritic Polyol

In some embodiments additive 107 may be a dendritic polyol. Dendritic polyols have a large number of active chemical sites and a flexible backbone. The dendritic polyols also have many hydroxyl groups which allow covalent linkage to the sol-gel matrix and act as highly functional crosslinkers.

In some embodiments, the dendritic polyol is Boltorn™ H2004 (CAS: 462113-22-0, Propanoic acid, 3-hydroxy-2-(hydroxymethyl)-2-methyl-,1,1′-[2-[[3-hydroxy-2-(hydroxymethyl)-2-methyl-1-oxopropoxy]methyl]-2-methyl-1,3-propanediyl] ester), as illustrated below:

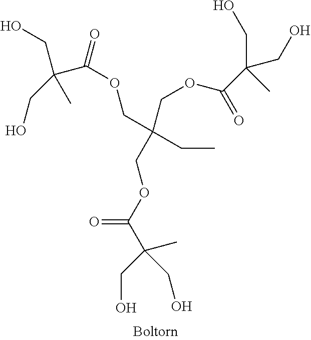 [SMILES: CCC(COC(=O)C(C)(CO)CO)(COC(=O)C(C)(CO)CO)COC(=O)C(C)(CO)CO]

In some embodiments Boltorn H2004 was added in a concentration of 1%-10% (w/w). The resulting hybrid sol-gel film had improved adhesion and flexibility compared to the hybrid sol-gel without Boltorn H2004.

Dendritic polyols may also be used when preparing a matrix using UV as detailed below.

### Polyvinylpyrrolidone

In some embodiments additive 107 may be Polyvinylpyrrolidone (PVP CAS: 9003-39-8) as illustrated below:

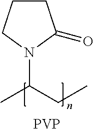

In some embodiments PVP was added in a molecular weight of 10 kDa and in a concentration of 5%-20% (w/w). The resulting hybrid sol-gel had improved adhesion and flexibility compared to the hybrid sol-gel without PVP.

In some embodiments a combination of two or more of PDMS, dendritic polyol and PVP may be used in the preparation of the epoxy silica ormosil solution.

In some embodiments, the combination is tuned to receive certain desired characteristics.

### Sol Gel and UV

In some embodiments, the sol-gel process may be followed by a UV curing process, with respect to some or all layers of film 130 (stage 626 in FIG. 11). In some embodiments a substrate is coated with the sol-gel solution followed by irradiation with UV light for curing. In some embodiments the coated substrate is then placed in an oven. In some embodiments the coated substrate is then cooled and is irradiated again with UV light for a final curing. UV curing following the sol-gel process may be faster than thermal curing and may allow an easier path to patterning.

In some embodiments, the UV process may be followed by thermal curing process, with respect to some or all layers of film 130. In some embodiments film 130 may comprise multiple layers wherein each layer may be cured by UV, by thermal curing or by a combination thereof; first by thermal curing followed by UV curing or first by UV curing followed by thermal curing. In some embodiments film 130 comprises 1-10 layers. In some embodiments film 130 comprises 1-100 layers. In some embodiments film 130 comprises 1-5 layers. In some embodiments film 130 comprises 10-20 layers. In some embodiments film 130 comprises 20-30 layers. U.S. Provisional Patent Application Nos. 62/593,936 and 62/613,085, and WIPO Publication No. WO 2018/042437 and U.S. Publication Nos. 2018/0072892 and 2018/0039131, incorporated herein by reference in their entirety, provide examples for illustrations of characteristics of formulations and films according to some embodiments of the invention—exemplifying the tuning of the emission spectrum (tuning of the emission peak is indicated by J) by adjusting formulation 74 and exemplifying the implementation of formulation 74 with two fluorescent compounds and different respective precursors.

### Film Preparation

Films 130 may be prepared from formulations 74 using a transparent substrate (e.g., glass, polyethylene terephthalate (PET), polycarbonate, poly-methyl-methacrylate (PMMA) etc.) or as stand-alone films (after solidification) and be used as color-conversion films in backlight units of displays. The substrate may be scrubbed to increase the surface roughness or be laminated to provide diffuser properties—in order to increase scattering or diffusing of blue light from the backlight unit.

In some embodiments, the surface of the substrate may be treated prior to applying the film. Treating the surface may improve the adhesion of the film and may prevent delamination and cracks at extreme conditions.

In some embodiments, the surface is treated by covalently binding aminosilanes. In one embodiment, the aminosilane is (aminoprpyl)triethoxysilane (APTES). The aminosilanes and APTES provide an anchoring active site for alkoxy condensation within the sol-gel reaction thus covalently binding the sol-gel matrix to the substrate and resulting in a strong adhesion between the film and the substrate. U.S. Provisional Patent Application Nos. 62/593,936 and 62/613,085, incorporated herein by reference in their entirety, provide examples for films prepared with pretreatment of the substrate with APTES.

In non-limiting examples, 0.1%-10% v/v of APTES were mixed with toluene. The mixture was then poured in to a bath. The substrate was dried with hot air and then placed in the bath with the mixture. The bath was then hermetically sealed (to prevent moisture absorbance) and the substrate was soaked for 3 hours. The substrate was then removed from the bath, washed with toluene and dried before coating.

The evaporation of alcohols 625 prior to the layer application may result in a denser sol-gel matrix which provides tight packaging of the RBF compound and accordingly may result in higher photostablity and therefore may reduce the number of layers. U.S. Provisional Patent Application Nos. 62/593,936 and 62/613,085, incorporated herein by reference in their entirety, provide a comparison of the normalized intensity in a single-color layer with and without evaporation.

Spreading formulation 74 may be carried out by any of manual coating (blade or spiral bar), automatic coting (blade or spiral bar), spin coating, deep coating, spray coating or molding; and the coatings may be applied on either side or both sides of the transparent substrate. Multiple layers of formulation 74 may be applied consecutively to film 130 (film thickness may range between 10-100 μm).

Concerning the drying, or curing process of formulation 74, it may be a two-step process comprising an initial short-term curing at a high reaction rate for determining the formation of the sol-gel matrix and a long-term curing at a lower reaction rate for determining the completion of the reaction (the temperature and duration of this step may be set to determine and adjust the reaction results). The initial short-term curing (drying) maybe carried out by a hot plate, an oven, a drier and/or an IR (infrared) lamp. In a non-limiting example, film 130 on glass may be placed on top of a hot plate or in an oven and undergo a heating profile: constant temperature (e.g., 60-100° C. for 1-3 hours) followed by step-wise temperature increments (e.g., 3-5 steps of 20-40° C. increase for 15-90 minutes each). In another non-limiting example, films may be cured by a drier or an IR lamp, e.g., being set on a conveyor (moving e.g., in 0.1-5 m/min) and heated to temperatures between 60-100° C. The curing may be configured to avoid film annealing and provide a required mesh size, while maintaining and promoting the stability of the RBF compound(s) 115. Curing parameters may be optimized with respect to a tradeoff between photostability and brightness, which relate to the film density resulting from the curing. In case of films with multiple layers (e.g., up to twenty layers), additional curing may be carried out between layer depositions (e.g., 50-90° C. for 1-3 hours) and a final curing may be applied after deposition of the last layer (e.g., 100-200° C. for 2-72 hours). In some embodiments, lower curing temperatures may be applied for longer times, e.g., the curing may be carried out for a week in 50° C. In some embodiments, curing temperatures may be raised stepwise, possibly with variable durations, e.g., the curing may be carried out stepwise at 30° C., 60° C., 90° C., two hours at each step. Optionally a final curing stage (e.g., at 130° C.) may be applied.

For example, green-fluorescent RBF compound in Z₃ (F₁TMOS) matrix was cured under different heat transport regimes: IR only (IR intensity 10%; 25 min on the conveyor moving at 0.1 m/min) dryer only (at consecutive 15 min steps of 30° C., 50° C., 70° C., 90° C., 110° C.) and a combination of IR followed by dryer, with a final curing of 24h in an oven at 130° C. The samples maintained their emission peaks, FWHM (full width at half maximum) and QY and exhibited the following reduction of emission intensity after eight days with respect to the initial intensity (measured by a fluorimeter): IR only—54%, dryer only—79%, IR and dryer—73%, showing the efficiency of the latter two methods.

The process may be further adjusted to yield encapsulation or bonding of the RBF compound(s) 115 in the matrix which narrows the FWHM of the emission band by adjusting the micro-environment of the fluorescent molecules. The process may be monitored and optimized using any of quantum yield measurements, fluorescent measurements, photometric measurements, photostability (lifetime) testing and others.

Concerning display properties, it is noted that emission peaks may be related to the display hue property and the FWHM may be related to the display saturation property. The adjustment of the hue and saturation properties may be carried out by corresponding adjustments in one or more components of formulation 74 and/or in the film production process described above. It is further noted that additional display properties such as intensity/lightness and brightness/LED power may be adjusted with respect to the designed film properties.

### Preparation and Measurement Details—Examples

The following illustrates some experimental procedures used to derive the results presented above (see FIG. 11 for overview). These experimental procedures are not limiting the application of the disclosed invention.

In a first example, film 130 was prepared by applying ten layers of formulation 74 with green-fluorescent RBF compound at a concentration of 0.1 mg/ml in the formulation, layer by layer, onto a transparent substrate and then applying two layers of formulation 74 with red-fluorescent RBF compound at a concentration of 0.05 mg/ml in the formulation, layer by layer, onto the former, green emitting layers. The inventors later found out that the multiple green-fluorescent layers may be replaced by fewer or even a single layer when evaporation of the alcohols is carried out prior to the layer application.

WIPO Publication No. WO 2018/042437 and U.S. Publication Nos. 2018/0072892 and 2018/0039131, incorporated herein by reference in their entirety, provide examples for the resulting spectrum and its emission peaks; examples for the larger color gamut range of film 130 in display 100 with respect to the standard LCD (sRGB) gamut and is in the range of the NTSC standard gamut; examples for multi-layered films 130 and their influence on the resulting spectrum, to demonstrate that the white point position may be tuned as desired by changing the structure of film 130, e.g., by adjusting the number of layers and/or concentration in formulation 74 of either RBF compound; and examples for various film compositions, such as 5- and 6-Carboxy X-rhodamine-Silylated illustrated below, as a non-limiting example. Similar covalent binding of RBF compounds 115 to the sol gel matrix may be achieved with other RBF compounds in similar ways.

### 5- and 6-Carboxy X-Rhodamine-Silylated

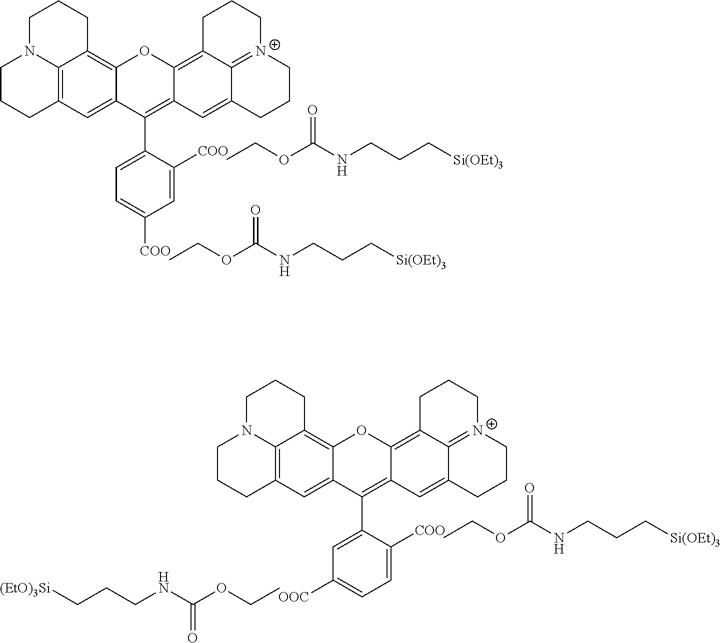

WIPO Publication No. WO 2018/042437 and U.S. Publication Nos. 2018/0072892 and 2018/0039131 provide multiple examples for film compositions and preparations methods, which are incorporated herein by reference in their entirety.
Cross-Linking with PMMA

Some embodiments comprise fluorescent compounds which are bonded to PMMA and have Si linkers to bond the PMMA-bonded compounds to the sol-gel matrix.

The following non-limiting examples illustrate binding RBF compounds to PMMA by showing the preparation of RBF compound ES-87 and cross-linking it with PMMA and linker of Si to be bonded to the sol-gel matrix. ES-86 was prepared as a precursor by dissolving 3-bromopropanol (0.65 ml, 7.19 mmol, 1 eq) in dry DCM (dichloromethane) under N₂ atmosphere. NEt₃ (0.58 ml, 7.91 mmol, 1.1 eq) was added and the mixture was cooled to 0° C. Acryloyl chloride (1.1 ml, 7.19 mmol, 1 eq) was added dropwise and the mixture was heated to room temperature and stirred at this temperature for 2 h. Upon completion, the mixture was quenched with 0.4 ml MeOH, diluted with DCM and was washed with saturated NaHCO₃. The organic layer was separated, dried with Na₂SO₄, filtered and the solvent was removed under reduced pressure. The crude product was purified by column chromatography (SiO₂, 10% EtOAc/Hex) to give the product as a colorless oil (943 mg, 68% yield).

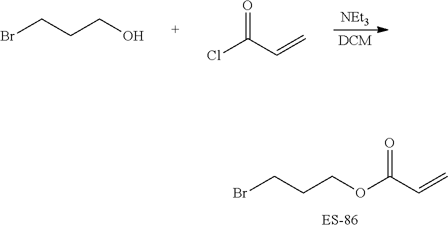

ES-87 was then prepared by dissolving RS-106 (see below, 150 mg, 0.26 mmol, 1 eq) in 3 ml dry DMF (dimethylformamide) under N₂ atmosphere. K₂CO₃ (55 mg, 0.4 mmol, 1.5 eq) was added and the mixture was stirred for 5 min before ES-86 (154 mg, 0.8 mmol, 3 eq) was added. The mixture was stirred for 3 hours at room temperature. Upon completion, the mixture was diluted with DCM and was washed with brine. The organic layer was separated, dried with Na₂SO₄, filtered and the solvents were removed under reduced pressure. The crude product was purified by column chromatography (SiO₂, DCM to 10% MeOH/DCM) to give the product as a blue powder (147 mg, 75% yield).

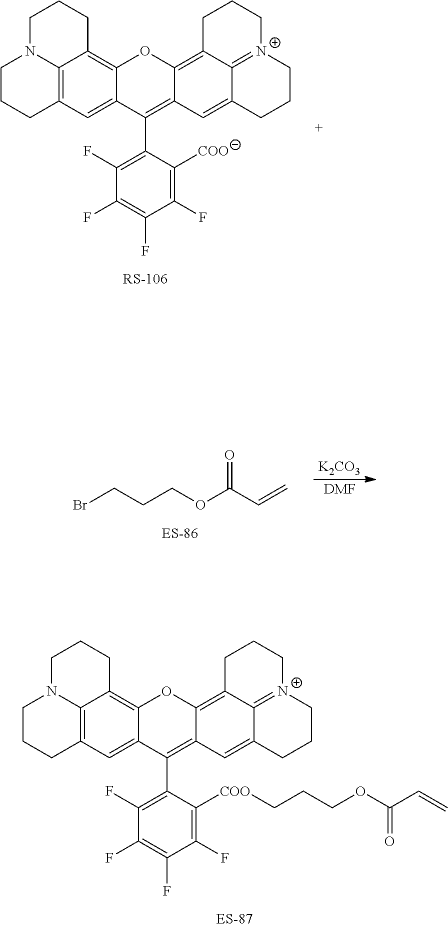

ES-87 was used to prepare cross-linked dyes as explained below in three non-limiting examples.

ES-91 was prepared by charging a 50 ml round-bottom flask with dry EtOH (9 ml) and N₂ was bubbled through for 20 min. Methyl methacrylate (0.3 ml, 2.8 mmol, 1 eq), ES-87 (4 mg, 0.0056 mmol, 0.002 eq) and AIBN (azobisisobutyronitrile, 10 mg, 0.056 mmol, 0.02 eq) were added and N₂ was bubbled through for 10 min. The reaction mixture was heated to reflux under N₂ atmosphere for 24 h. Upon completion, the mixture was cooled to room temperature and was evaporated to dryness under reduced pressure. The crude product was dissolved in 3 ml of DCM and then was added dropwise to 50 ml of Hex. The precipitate was filtered and the purification process was repeated again to give the product as a blue powder.

ES-99 was prepared by charging a 50 ml round-bottomed flask with dry EtOH (9 ml) and N₂ was bubbled through for 20 min. Methyl methacrylate (0.3 ml, 2.8 mmol, 1 eq), 3-methacryloxypropyl trimethoxysilane (34 μl, 0.14 mmol, 0.05 eq), ES-87 (8 mg, 0.01 mmol, 0.002 eq) and AIBN (10 mg, 0.056 mmol, 0.02 eq) were added and N₂ was bubbled through for 10 min. The reaction mixture was heated to reflux under N₂ atmosphere for 24 h. Upon completion, the mixture was cooled to room temperature and was evaporated to dryness under reduced pressure. The crude product was dissolved in 3 ml of DCM and then was added dropwise to 50 ml of Hex. The precipitate was filtered and the purification process was repeated again to give the product as a blue powder.

ES-113 and ES-110 were prepared similarly to ES-99, but using higher concentration of the linker 3-methacryloxypropyl trimethoxysilane, namely 50% and 100% linker respectively, compared with 5% in ES-99. WIPO Publication No. WO 2017/085720 and U.S. Pat. No. 9,868,859, incorporated herein by reference in their entirety, some embodiments of PMMA cross-linked dyes, according to some embodiments of the invention. Any of disclosed RBF compounds 115, as well as other dyes such as assistant dyes 117 may be cross linked to one or more polymers in color conversion film 130.

### Protective Films

Some embodiments comprise applying a protective film 131 to color conversion film 130 and/or configuring color conversion film 130 to have protective properties which prevent humidity damages and cracking. Any type of color conversion film 130 may be protected and/or enhanced as described in the following, e.g., RBF-compounds-based films 130 as well as films 130 based on other organic or inorganic fluorescent molecules and quantum-dot-based color conversion films 130.

For example, protective film 131 may be formed using zirconium-phenyl siloxane hybrid material (ZPH), a transparent, clear and flexible polymer, based on the description in Kim et al. 2014 (“Sol-gel derived transparent zirconium-phenyl siloxane hybrid for robust high refractive index led encapsulant”, ACS Appl. Mater. Interfaces 2014, 6, 3115-3121), with the following modifications, found by the inventors to isolate films 130 from the surroundings, provide the film mechanical support and prevent cracks.

ZPH is a silica based polymer gel, cured in a hydrosilylation addition reaction. The polymer comprises two resin components: HZPO (a Si—H functionalized silica) and VZPO (a vinyl functionalized silica). Both components are synthesized in a sol-gel reaction separately and then mixed in the proper ratio into formulation 74 and cured to yield a semi-solid form. HZPO was mixed from 3.2 ml Methyldiethoxysilane (MDES), 6.5 g diphenylsilanediol (DPSD) and 25 mg amberlite IRC76 for 1 hour at 100° C. and then, while stirring, 673 μL zirconium propoxide (ZP) 70% in 1-propanol was added slowly and the reaction continued overnight. VZPO was mixed from 3.1 g vinyltrimethylsilane (VTMS), 4.4 g DPSD and 7.7 mg barium hydroxide monohydrate in 0.86 ml p-xylene at 80° C. and then, while stirring, ZP was added slowly, with the reaction time being four hours. ZPH was prepared by mixing VZPO and HZPO in a ratio of 1:1 mol/mol and 10 ml of a platinum catalyst was added to the viscous liquid, which was then stirred vigorously for one minute and applied on the substrate using a coating rod. Protective film 131 was inserted into the oven in 150° C. for three hours for curing.

Additional examples for protective films 131 include using polymerized MMA (methyl-methacrylate) as protection, by allowing MMA to diffuse into the sol-gel pores. Color conversion films 130 may be coated with additional MMA monomers that penetrate the sol-gel pores and then polymerize inside, thereby improving the life time of film 130. The preparation procedure may be modified to provide such polymerization conditions.

Some embodiments comprise using a trimethoxysilane derivative as coating, e.g., an R-TMOS coating with R being e.g., phenyl, methyl, CH₂CH₂CF₃ or other groups, with proper process adaptations which provide the coating conditions for forming protective film 131 and/or protective characteristics of film 130.

Some embodiments comprise using as epoxy silica ormosil solution layer as protective coating 131, such as epoxy silica ormosil solution with no dye as protective layer 131 applied on cured film 130. Other protective coatings 131 of film 130 may comprise an acetic anhydride surface treatment derived from acetic acid with ending —OH groups changed to -Ac groups to enhance life time and/or chlorotrimethoxysilane protective layer 131 having endings with —OH groups modified to -trimethylsilane to enhance life time.

In certain embodiments, disclosed protective films 131 may be used in a range of applications for protective respective films from humidity and mechanical damages. For example, disclosed protective films 131 may be used to coat various plastic films (made of e.g., PEI (polyethylenimine), acrylic polymers, polycarbonate, PET, PDMS (polydimethylsiloxane) and related siloxanes, as well as other polymers), glass and metals/metal oxide films or surfaces (e.g., of copper, silicon, silicon oxides, aluminum, titanium and other transition metals and their oxides). Protective films 131 may be configured to have corresponding good adhesion to the respective films.

In some embodiments, protective films 131 may be used to coat diffusers, polarizers, glasses or any other film that needs temperature and humidity protection (e.g., up to 85° C., 95% relative humidity).

In some embodiments, protective films 131 and/or formulations thereof may be used as fillers in porous films.

### UV Curing Processes

UV curing processes 740 may be used additionally or in place of sol gel processes to provide the color conversion films. Formulations with and without rhodamine-based fluorescent compounds, films, displays and methods are provided, in which the fluorescent compounds are stabilized and tuned to modify display backlight illumination in a manner that increases the display's efficiency and widens its color gamut. UV cured formulations may be used to provide fluorescent films that may be applied in various ways in the backlight unit and/or in the LCD panel and improve the display's performance. The formulation, curing process and film forming procedures may be optimized and adjusted to provide a high photostability of the fluorescent compounds and narrow emission peaks of the backlight unit.

In certain embodiments, the sol gel process may be replaced by a UV curing process, with respect to some or all layers of film 130. Similar or different RBF compounds 115 may be used in UV cured layers, such as RBF compounds disclosed above, and films 130 produced by UV curing may replace (or complement) films 130 (or layers 132 and/or 134) produced by the sol gel processes in the configurations of backlight unit 300 and display 100 which are illustrated herein. Other organic or inorganic fluorescent dyes as well as quantum dots may be embedded in disclosed UV cured films 130 or modifications thereof as well. Also, configurations of film 130 disclosed above in relation to display configurations, polarizing films and red enhanced films may be implemented with UV cured films 130 or layers 132, 134. In the following, examples for applicable UV processes are presented.

In some embodiments, UV curing is advantageous due to the wide range of UV curable materials, which provide an opportunity to create polymeric matrices which are compatible with the incorporated dyes, such as RBF compounds 115. In order to achieve maximal life time and QY, the structure and the crosslinking density may be optimized and the interaction between the dye and the matrix may be minimized. The use done in UV curing of highly reactive components may significantly reduce the amount of non-crosslinked material even at low UV exposure and short retention time—thereby enabling to minimize damage to the dye molecules while providing required matrices for the dye, e.g., matrices which provide high photostability, narrow FWHM (e.g., 40-60 nm) and high QY in the green and red regions (e.g., due to less occupied vibration levels), for RBF compounds 115 or other fluorescent molecules). The cross-linking degree may be optimized per dye material in order to obtain high QY (too much cross linking may degrade the QY).

Various examples are presented below for formulations 74 which are then UV cured after being applied to transparent PET (polyethylene terephthalate) substrate or diffuser films (PET coated with PMMA coating) by drawing using coating rods for providing films with widths ranging 20-100 which are then irradiated once under “H” UV lamp at conveyor speed 2-7 m/min. Color conversion films 130 may comprise multiple layers which may be applied one on top of the other. Resulting color conversion films 130 (or protective films 131, see below) may be used as explained above by themselves or in combination with films 130 produced by sol gel processes 600. Formulations 74 for UV cured films 130 may comprise RBF compounds 115 as described above. Life times of fluorescent dyes in UV cured matrix are different for different dyes and depend on the cured formulation and on the curing conditions. Generally, the stability of RBF compounds 115 under continued blue light excitation provides a long life time.

UV cured films 130, in particular UV cured color conversion films 130, may be prepared from formulations 74 comprising 65-70% monomers, 25-30% oligomers, and 1-5% photoinitiator; as well as color conversion elements such as RBF compounds at low concentration (e.g., 0.005-0.05%), in weight percentages of the total formulation. Following are non-limiting examples for such formulations 74, which are UV cured to yield respective films 130.

WIPO Publication No. WO 2018/042437 and U.S. Publication Nos. 2018/0072892 and 2018/0039131 provide examples for UV cured films, their preparation methods and resulting film performance, and are incorporated herein by reference in their entirety.

The produced films may be combined and optimized to form film 130, for example a non-limiting example of film 130 was optimized to operate with a blue backlight source 80A of about 10 mW/cm² of optical power and provided a red emission peak at 616 nm with FWHM of 60 nm and a green emission peak at 535 nm with FWHM of 45 nm, with a white point at (0.30, 0.27) CIE 1931 coordinates (white point adjustment may also be carried out as disclosed above). WIPO Publication No. WO 2018/042437 and U.S. Publication Nos. 2018/0072892 and 2018/0039131, which are incorporated herein by reference in their entirety, further provide examples for the resulting absorption and emission spectra of film 130 and example for color gamut ranges which are comparable or surpass respect to sRGB, NTSC and quantum-dots-based displays in performance parameters.

### Protective Films

Some embodiments comprise applying a protective film 131 to color conversion film 130 and/or configuring color conversion film 130 to have protective properties which prevent humidity damages and cracking. Any type of color conversion film 130 may be protected and/or enhanced as described in the following, e.g., RBF-compounds-based films 130 as well as films 130 based on other organic or inorganic fluorescent molecules and quantum-dot-based color conversion films 130.

For example, UV cured protective film 131 may be formed using a mixture of 3,4-epoxycyclohexylmethyl 3,4-epoxycyclohexanecarboxylate, triarylsulfonium hexafluoroantimonate salts, mixed-50 wt % in propylene carbonate, polyether modified polydimethylsiloxane and 3-ethyloxetane-3-methanol, which is UV cured on a conveyor.

In another example, UV cured protective film 131 may be formed by mixing 76.8% 3,4-epoxycyclohexylmethyl 3,4-epoxycyclohexanecarboxylate, 19.2% trimethylolpropane (TMP) oxetane (TMPO), 3.8% triarylsulfonium hexafluoroantimonate salts, mixed-50 wt % in propylene carbonate and 0.2% polyether-modified polydimethylsiloxane (in this order) and stirring the mixture at room temperature. The sample was applied to a sol-gel layer (e.g., color conversion film 130 produced by a sol gel process disclosed above) by drawing using a coating rod to form a 50 μm layer and then irradiated once under H UV lamp at conveyor speed 7 m/min. The sol-gel layer was cleaned with ethanol and air dried before coating.

### BLU Modifications and Configurations

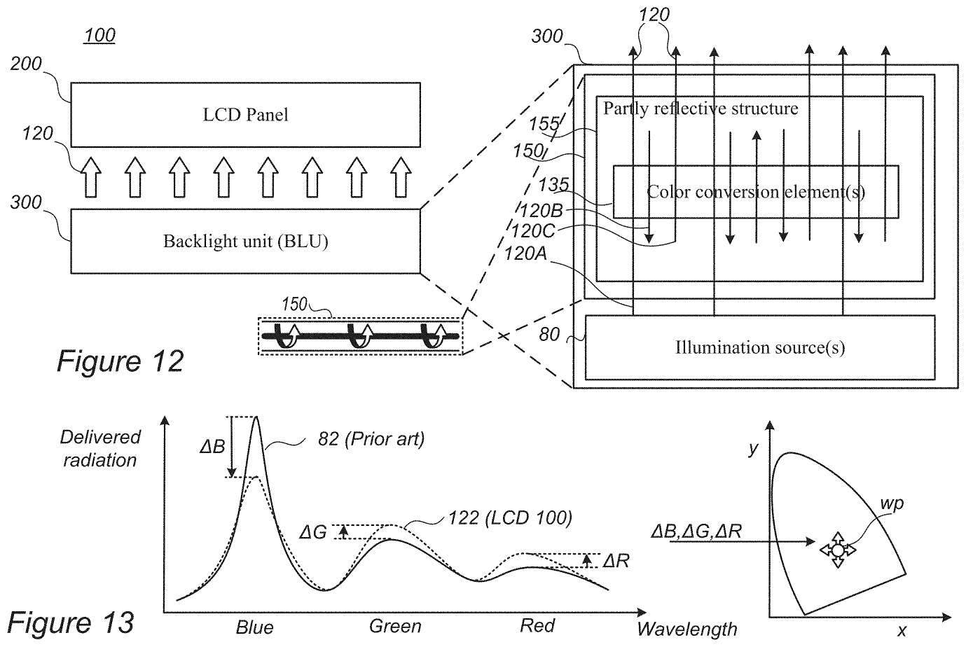

FIG. 12 is a high-level schematic illustration of a backlight unit (BLU) 300 for a liquid crystal display (LCD) 100, according to some embodiments of the invention. BLU 300 comprises at least one color conversion unit 150 and at least one illumination source 80.

Color conversion unit(s) 150 comprises at least one partly reflective structure 155 and at least one color conversion element 135. Color conversion element(s) 135 comprises at least one fluorescent dye (e.g., RBF compound(s) 115) configured to convert blue radiation to green and/or red radiation. Illumination source(s) 80 is configured to deliver radiation through color conversion unit(s) 150 to a LCD panel 200 of LCD 100. Partly reflective structure(s) 155 is configured to redirect at least a part of the delivered radiation to pass multiple times through color conversion element(s) 135. Radiation (denoted by 120A) is delivered by illumination source(s) 80 through partly reflective structure(s) 155 which redirects (e.g., any of reflects, scatters, disperses, etc.) radiation through color conversion element(s) 135, denoted as radiation 120B, 120C (for multiple, two or more passes through color conversion element(s) 135) before radiation 120 is delivered to LCD panel 200. Partly reflective structure(s) 155 may be configured to recycle blue radiation through color conversion unit(s) 150 to enhance color conversion efficiency and/or flux, and/or to increase the lifetime of color conversion element(s) 135. Advantageously, the inventors have found out that passing the radiation from illumination source(s) 80 multiple times through color conversion element(s) 135 provides illumination radiation 120, modifies the white point of LCD 100 in a controllable manner, and one color conversion unit(s) may be configured to set a white point of delivered radiation 120 and LCD 100 according to specified requirements.

FIG. 13 is a high-level schematic illustration of white point adjustment for LCD 100, according to some embodiments of the invention. FIG. 13 illustrates schematically a prior art spectrum 82 of delivered radiation, and a spectrum 122 of delivered radiation 120 to LCD panel 200, according to some embodiments of the invention of delivered radiation. Spectra 82, 122 have a relatively large blue peak (e.g., between 440-460 nm) and smaller green and red peaks (e.g., between 520-540 nm and between 610-630 nm, respectively). However, multiple passing of the delivered radiation resulting in spectrum 122 has a lower blue peak and higher green and/or red peaks than prior art spectrum 82, due to enhanced color conversion resulting from the multiple passes. Lowering the blue peak (indicated schematically by ΔB) and raising the green and/or red peaks (indicated schematically by ΔG and ΔR, respectively) changes the white point (indicated schematically as wp on the schematic color space diagram) of LCD 100 with respect to prior art LCDs, and configuration of partly reflective structure(s) 155 and color conversion element(s) 135 provides required or specified settings of the white point by increasing or decreasing ΔB, ΔG and ΔR according to specifications (indicated schematically by the arrows).

FIGS. 14A-F are high-level schematic illustrations of BLUs 300, according to some embodiments of the invention. One or more illumination source(s) 80, such as blue LEDs 80A and/or white LEDs 80B in various configurations may be used with a waveguide 420 for guiding the radiation from illumination source(s) 80 towards partly reflective structure(s) 155 and/or towards optical element(s) 410 guiding delivered radiation 120 towards LCD panel 200. For example, optical element(s) 410 may comprise polarizer(s), dual brightness enhancement film(s) (BEF), possibly dual brightness enhancement film(s) (DBEF) etc.

It is noted that while the numerals 300 and 400 are used herein to denote the BLU and the modified illumination sources, respectively, certain embodiments comprise extended illumination sources 400 which may be used as BLUs 300. In such cases, either numeral, 300 or 400, is applicable. In other cases, with BLU 300 comprising additional layers 350, the numerals are clearly distinct. It is further noted that various embodiments may comprise combinations of illumination source 400 and layers 350, which are disclosed in different embodiments.

Partly reflective structure(s) 155 may be configured in different ways, e.g., comprising a top reflector 154 configured to re-introduce at least part of radiation 120A back into color conversion element(s) 135 as radiation 120B (see FIG. 12) and possibly comprising a bottom reflector 152 configured to re-direct at least part of radiation 120B towards LCD panel 200 as radiation 120C, possibly back through color conversion element(s) 135. Top reflector 154 is partly reflective, comprising e.g., a perforated reflector 158 (e.g., as illustrated schematically in FIG. 14A), a spectral filter having specified transmittance and reflectance curves configured to pass and/or reflect part of the blue radiation into partly reflective structure(s) 155 and partly pass red and/or green radiation out of partly reflective structure(s) 155. The parts of radiation in different wavelength ranges may be configured according to required intensity of B, G and R and according to the geometric configuration of BLU 300 and of partly reflective structure(s) 155. For example, top reflector 154 may comprise one or more short pass filter(s) (SPFs), long pass filter(s) (LPFs). In certain embodiments, top reflector 154 may further comprise diffusing elements such as diffuser layer(s) and/or may be patterned to control and homogeneity of radiation 120. In certain embodiments, intermediate filter layers 156 may be further introduced between color conversion element(s) 135 to regulate radiation passing therebetween (see e.g., FIG. 14D). In certain embodiments, multiple lateral illumination sources 80A may be used (see e.g., FIG. 14B), with only some of the illumination radiation passing through color conversion element(s) 135, e.g., 135R and 135G, in order to increase their lifetime. For example, blue light from upper illumination source 80A may be provided without any color conversion, while blue light from bottom illumination source 80A may be mostly converted into red and green light. Additional reflectors and/or diffusers 445, 447 may be positioned, e.g., below partly reflective structure(s) 155 such as in association with waveguide 420 (e.g., see FIG. 14C) and/or on sides of waveguide 420 and/or color conversion unit(s) 150 (e.g., see FIGS. 14A-F). BLU 300 may further comprise additional reflectors 440 (see e.g., FIGS. 14C, 14D) as well as sides of waveguide 420 and/or sides of partly reflective structure(s) 155, which may be configured to control the dispersal of radiation within BLU 300, to yield required illumination parameters for delivered radiation 120. In certain embodiments, no diffusers are used in color conversion unit(s) 150.

Color conversion element(s) 135 may comprise one or more layers, e.g., color conversion element(s) 135G configured to convert blue radiation into green radiation, color conversion element(s) 135R configured to convert blue and/or green radiation into red radiation, in various spatial configurations, such as one or more layers of each, combined layers, separate elements within partly reflective structure(s) 155 etc. In various embodiments, color conversion element(s) 135G may be set between color conversion element(s) 135R (see e.g., FIG. 14A) and/or color conversion element(s) 135R may be set between color conversion element(s) 135G and/or color conversion elements 135R, 135G may be integrated into a single layer (see e.g., FIG. 14E). Color conversion element(s) 135 may use rhodamine-based fluorescent dyes, embedded in various matrices such as sol-gel based matrices, UV cured matrices, etc.—as disclosed e.g., in WO 2018/042437 and U.S. Publication Nos. 2018/0072892, 2018/0037738 and 2018/0039131, or may be based on other color conversion elements, such as quantum dots. For example, the fluorescent dye(s) may be configured to convert blue radiation to green radiation, green radiation to red radiation and/or blue radiation to red radiation.

In certain embodiments, color conversion element(s) 135 may comprise assistant dyes configured to modify the radiation spectrum by absorbing radiation in one specified wavelengths range and emitting radiation at another one specified wavelengths range, which is typically lower the absorption range, at efficiencies typically ranging between 70-90%. For example, assistant dyes may be selected or configured to absorb radiation outside the range of the LCD filters (B, G and R) and emit radiation within such range, to enhance display efficiency, as disclosed, e.g., in U.S. Publication No. 2018/0039131. For example, the fluorescent dye(s) may be configured to convert radiation outside a wavelength range of a color filter of the LCD into radiation inside the wavelength range of the color filter of the LCD.

Illumination source(s) 80 may comprise any of blue LEDs 80A and/or white LEDs 80B (see e.g., FIG. 14E), combinations thereof, as well as illumination source(s) 80 having any other combination of colors.

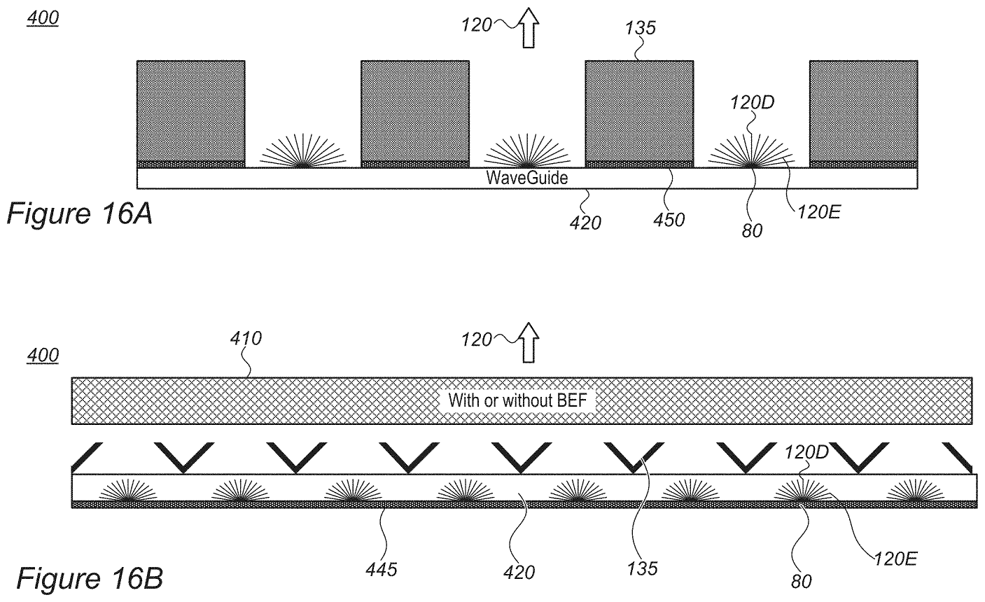

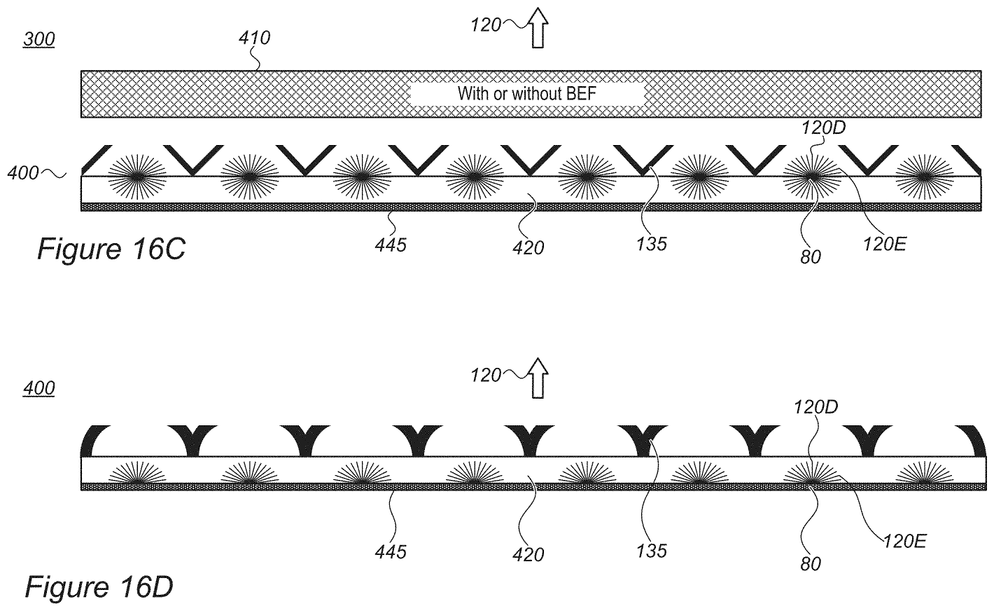

FIGS. 15A-15E and 16A-16D are high-level schematic illustrations of BLUs 300 having color conversion unit(s) 150 in which color conversion elements 135 receive only part of the overall radiation, according to some embodiments of the invention. In certain embodiments, illumination source(s) 80 may be further configured to deliver only some of the radiation through color conversion unit(s) 150 to LCD panel 200, and deliver a part of the radiation directly to LCD panel 200. Embodiments illustrated in FIGS. 15E and 16A-16D may comprise color conversion unit(s) 150 disclosed above.

For example, FIG. 15A illustrates schematically BLU 300 having one or more illumination source(s) 80B configured to deliver radiation to LCD panel 200 through color conversion unit(s) 150 (shown very schematically) and one or more illumination source(s) 80A configured to deliver radiation directly to LCD panel 200.

In various embodiments, illustrated schematically in FIGS. 15B and 15C, dedicated illumination source(s) 80A, 80B may be configured to deliver radiation through separate paths to provide delivered radiation 120 (e.g., through different waveguides 420, 430 as illustrated schematically in FIG. 15B) and/or illumination source(s) 80 may be configured to deliver radiation which is then split in waveguide 420 so that some of the radiation is delivered directly to LCD panel 200 and some is delivered through corresponding color conversion elements 135R, 135G, possibly through corresponding elements 403R, 403G (radiation indicated by 80A, 80B, respectively, in schematic illustration FIG. 15C), which may comprise any of color filters, reflectors, diffusers etc. Such elements are optional and may be set above and/or below respective color conversion elements 135R, 135G.

FIGS. 15D and 15E illustrate schematically separation of radiation from illumination source(s) 80A, 80 into direct radiation 120D and color converted radiation 120E in two conceptual configurations, which may be used separately or be combined. In the configuration illustrated schematically in FIG. 15D, the splitting of the delivered radiation into direct radiation 120D and color converted radiation 120E is carried out globally at the BLU level, e.g., by directing only some of the radiation into color conversion unit(s) 150 and letting a part of the radiation exit BLU 300 directly. Any configuration of BLU 300 illustrated e.g., in FIG. 14A-F may be used for such configurations. In the configuration illustrated schematically in FIG. 15E, the splitting of the delivered radiation into direct radiation 120D and color converted radiation 120E is carried out locally, by configuring color conversion elements 135 to spatially intercept only part of the radiation 120E delivered by illumination source(s) 80, and not affecting some of the delivered radiation 120D at all. For example, in the side walls configuration illustrated schematically in FIG. 15E, color conversion elements 135 intercept only side lobes of radiation emitted from illumination source(s) 80, the amount of which is geometrically controlled and also depends on the configuration of illumination source(s) 80 and the radiation distribution they emit.

In certain embodiments, direct illumination (as illustrated schematically in FIG. 15E) and indirect illumination (as illustrated schematically in FIG. 15D, e.g., using diffusive elements) may be combined in BLU 300 to optimize performance such as color conversion efficiency and lifetime.

FIGS. 16A-16D illustrate schematically various configurations of BLUs 300 with color conversion elements 135 that intercept only side lobes 120E of radiation emitted from illumination source(s) 80, leaving a predefined part 120D of the illumination to be delivered without passing through any color conversion elements 135. For example, FIG. 16A illustrates schematically color conversion elements 135 as lateral walls, which may be configured to have various thicknesses and heights according to the required amount of color conversion and radiation side lobes 120E. It is noted that in FIGS. 15E and 16A color conversion elements 135 may receive and convert radiation coming from either side of the respective walls, enhancing color conversion efficiency. Color conversion elements 135 may further comprise reflectors 450 to enhance color conversion efficiency.

In certain embodiments, illustrated schematically in FIGS. 16B-16D, color conversion elements 135 may be designed in various geometric and spatial configurations, to determine and control the relative part of the radiation which passes therethrough, and at least partly converted to green and/or red radiation by respective dyes in color conversion elements 135. For example, color conversion elements 135 may be designed as truncated triangles, or zig-zags (illustrated e.g., in FIGS. 16B and 16C), as truncated domes (illustrated e.g., in FIG. 16D) or any other shape having openings or perforations through which part 120D of the radiation may be delivered to LCD panel 200 in radiation 120 without passing through color conversion elements 135. The size and arrangement of the openings between color conversion elements 135 and the spatial design of color conversion elements 135 may be configured to control the radiation flux directed to color conversion elements 135 to optimize performance such as color conversion efficiency and lifetime.

In any of the disclosed embodiments, optical element(s) 410 such as BEF(s), DBEF(s), diffuser(s), polarizer(s) etc., may be used on top of BLU 300 to further configure delivered radiation 120 according to given requirements (e.g., have a Lambertian distribution with specified parameters). In any of the disclosed embodiments, illumination source(s) 80 may comprise any of multiple LEDs and/or multiple dispersive and/or diffusive elements in optical communication with waveguide 420 delivering radiation from LED(s). Illumination source(s) 80 may comprise scattering spots configured to deliver radiation received from edge LEDS through the waveguide—in the direction of the color conversion elements and eventually the LCD panel. The dispersive and/or diffusive elements may be set at a top and/or at a bottom layer of waveguide 420 (see e.g., FIGS. 16C and 16D, respectively), and may be configured to deliver a given profile of radiation, to control and determine parts 120D, 120E thereof. In any of the disclosed embodiments, dispersion of the fluorescent dyes through color conversion elements 135 may be configured to enhance lifetime, e.g., change gradually in fluorescent dye concentration.

The inventors have found out that when color conversion efficiency is high, it may be advantageous to reduce the radiation flux passing through color conversion elements 135, in order to increase their lifetime, and/or to reduce the overall flux of radiation used in LCD 100, to increase the lifetime thereof and of elements in LCD panel 200. Enhanced color conversion efficiency may be used to reduce the clue radiation flux delivered to color conversion unit(s) 150, and to deliver blue radiation separately from color converted radiation to improve efficiency, lifetime and enhance the ability to control the white point of LCD 100. In certain embodiments, the inventors have noted that recycled blue radiation, passing multiple times through color conversion unit(s) 150, degrades color conversion elements 135 less than a larger flux of non-recycled radiation. Combining shaped color conversion elements 135 with elements of partly reflective structure(s) 155 may be used to simultaneously enhance color conversion efficiency, increase lifetime and provide controllable white point of delivered radiation 120 and of LCD 100.

In any of the embodiments disclosed in FIGS. 15A-15C, 15E and 16A-16D, partly reflective structure(s) 155 are not illustrated for simplicity reasons and may well be integrated in the illustrated BLUs according to the principles outlined above (see e.g., FIGS. 13A, 14A-F and 15D).

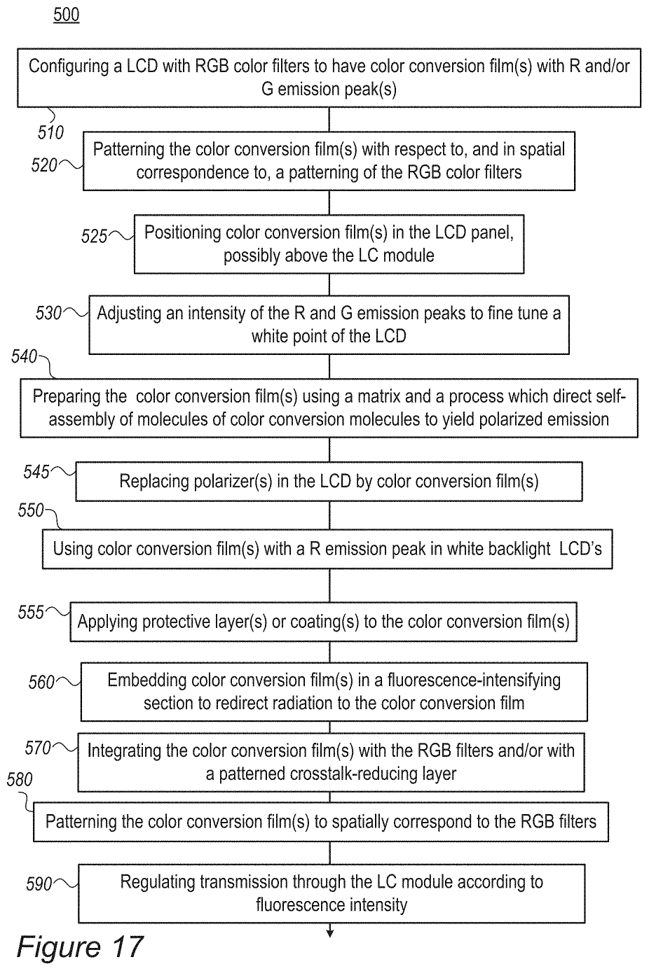

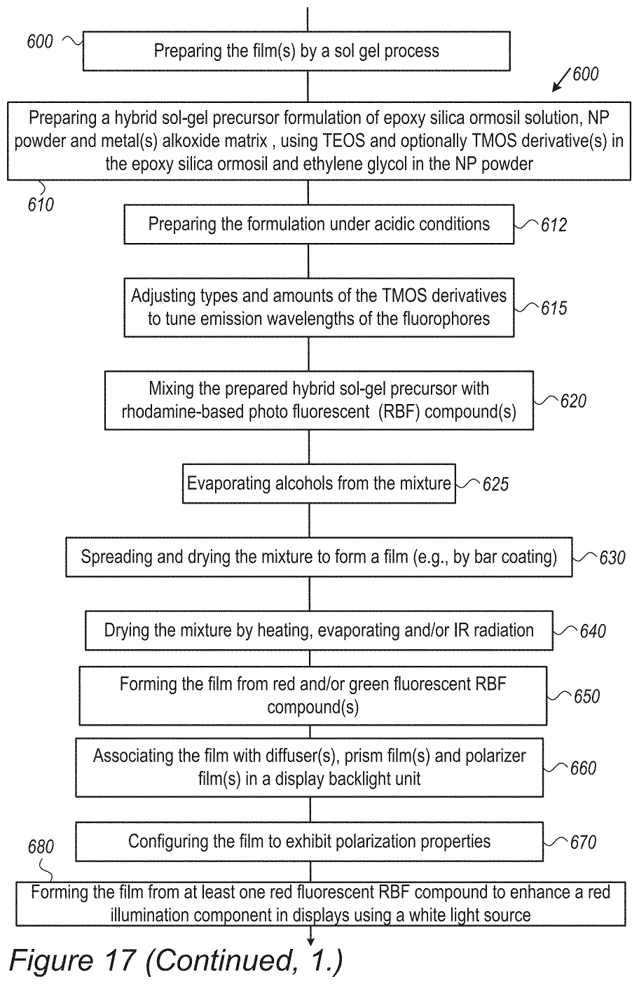

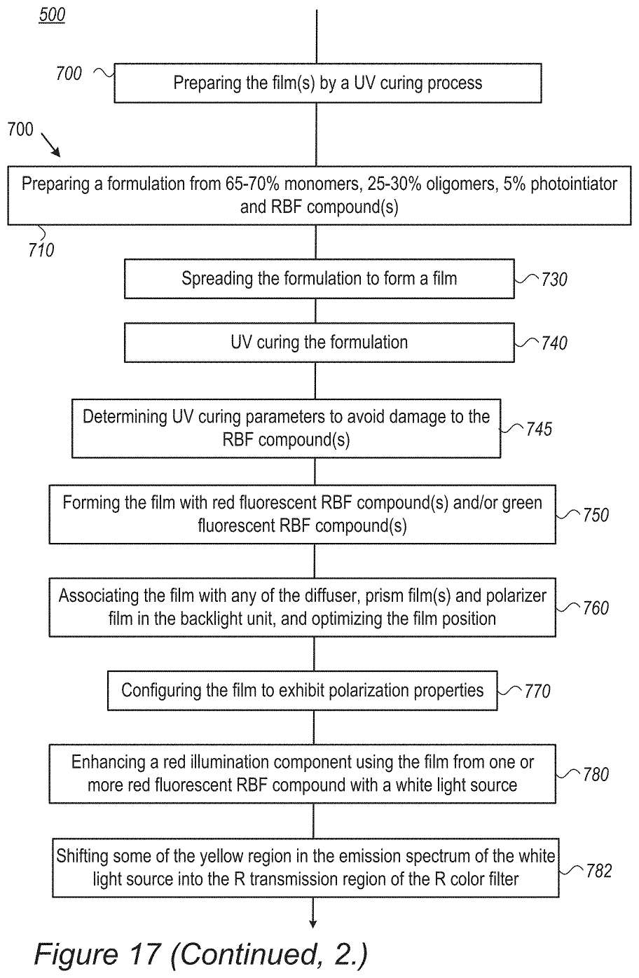

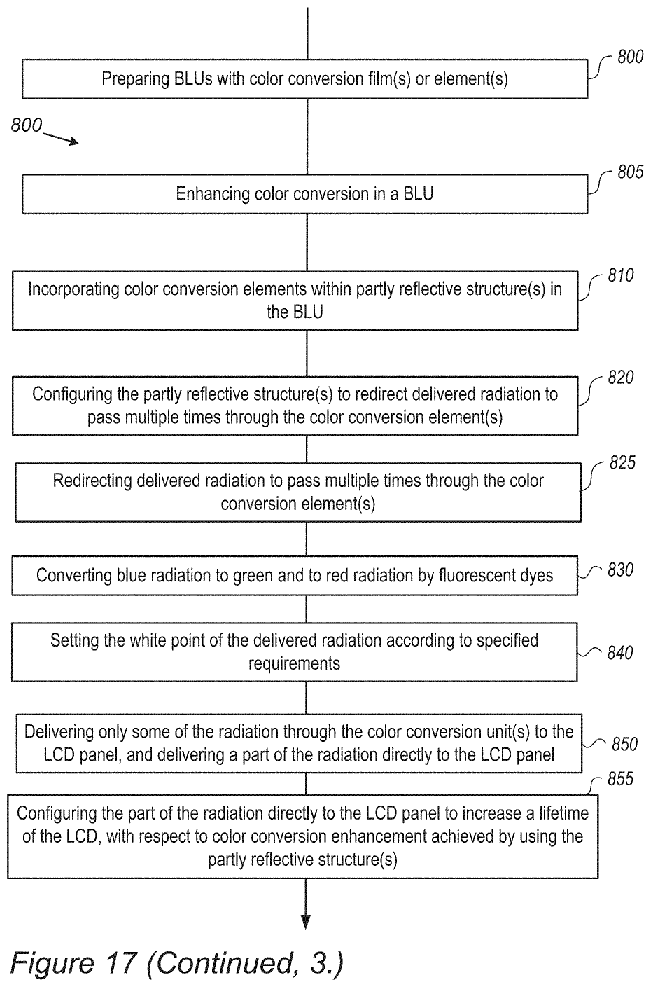

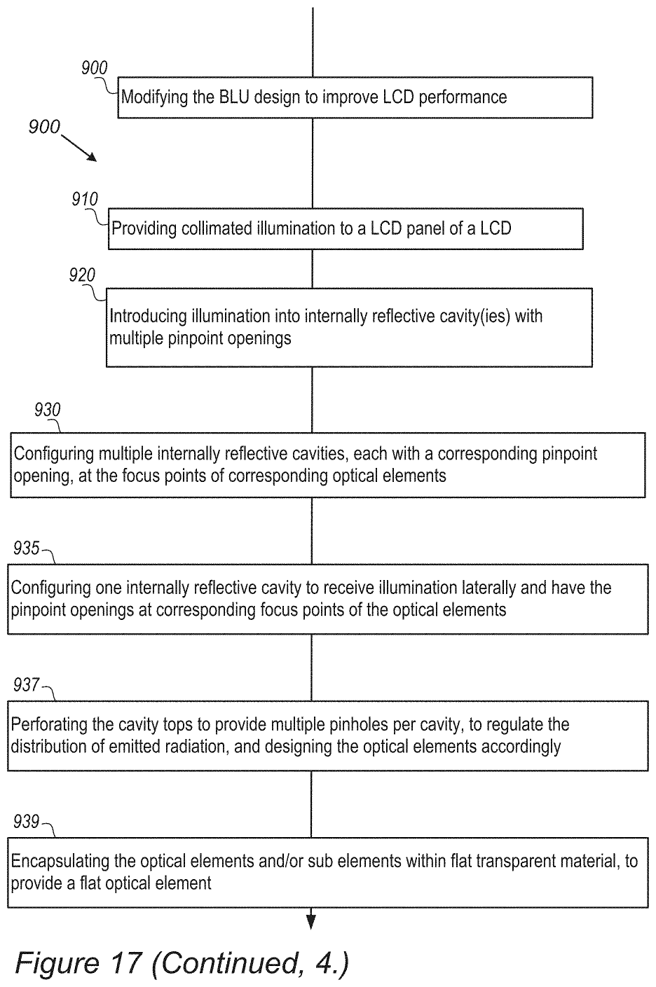

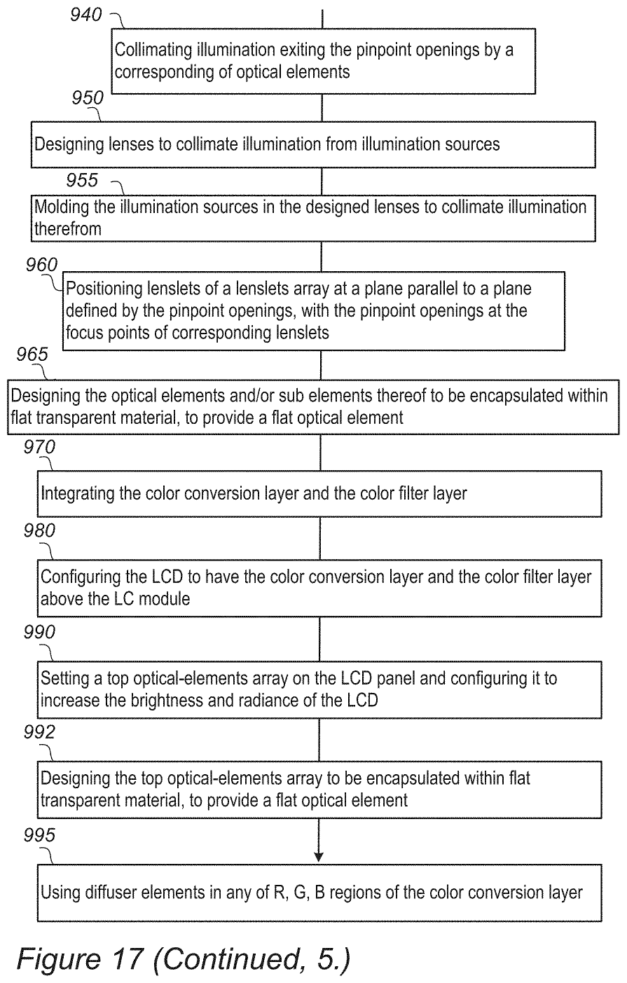

Various embodiments of disclosed methods are presented in FIG. 17 below, the stages of which may be combined into various embodiments.

FIG. 17 is a high-level flowchart illustrating a method 500, according to some embodiments of the invention. The stages of method 500 may be carried out with respect to various aspects of precursors 72, formulations 74, films 130 and displays 100 described above, which may optionally be configured to implement method 500, irrespective of the order of the stages.

In some embodiments, method 500 comprises configuring a LCD with RGB color filters to have at least one color conversion film prepared to have a R emission peak and/or a G emission peak (stage 510), patterning the at least one color conversion film with respect to a patterning of the RGB color filters to yield a spatial correspondence between film regions with R and G emission peaks and respective R and G color filter (stage 520), and positioning the color conversion film in an LCD panel of the LCD, possibly above the LC module (stage 525).

In some embodiments, method 500 comprises configuring a LCD with RGB color filters to have at least one color conversion film prepared to have a R emission peak and a G emission peak (stage 510), and adjusting an intensity of the R and G emission peaks of the at least one color conversion film to fine tune a white point of the LCD to be at a center of an expected line of deterioration of the intensity within given LCD specifications (stage 530).

In some embodiments, method 500 comprises configuring a LCD with RGB color filters to have at least one color conversion film prepared to have a R emission peak and a G emission peak (stage 510), preparing the at least one color conversion film using a matrix and a process which direct self-assembly of molecules of color conversion molecules of the at least one color conversion film to yield polarization of at least part of illumination emitted by the color conversion film (stage 540), and replacing at least one polarizer in the LCD by the at least one color conversion film (stage 545).

In some embodiments, method 500 comprises configuring a LCD with RGB color filters and white backlight illumination to have at least one color conversion film prepared to have a R emission peak (stage 550).

In some embodiments, method 500 further comprises applying a protective layer to the color conversion film (stage 555). For example, method 500 may further comprise any of: preparing the protective layer by a sol gel process with at least one of: zirconium-phenyl siloxane hybrid material (ZPH), methyl methacrylate (MMA), trimethoxysilane derivative and an epoxy silica ormosil solution; preparing the protective layer by an acetic anhydride surface treatment and/or a trimethylsilane surface treatment; and/or preparing the protective layer by a UV curing process using a mixture of 3,4-epoxycyclohexylmethyl 3,4-epoxycyclohexanecarboxylate and triarylsulfonium hexafluoroantimonate salts, mixed in propylene carbonate.

The at least one color conversion film may comprise at least one RBF compound defined by Formula 1 and/or Formula 2.

Method 500 may further comprise embedding the at least one color conversion film in a fluorescence-intensifying section which comprises at least one supportive structure configured to redirect radiation to the at least one color conversion film (stage 560).

Method 500 may further comprise integrating the at least one color conversion film with the RGB color filters and/or with a crosstalk-reducing layer comprising a structural framework which is patterned according to a pixel structure of the RGB color filters (stage 570).

Method 500 may further comprise patterning the at least one color conversion film to yield a spatial correspondence between film regions with R and G emission peaks of the at least one color conversion film and respective R and G color filters (stage 580).

Method 500 may further comprise regulating transmission through the LC module according to an intensity of fluorescence from the at least one color conversion film, by tuning down the transmission through the LC module when the at least one color conversion film is fresh and provides a high level of fluorescence, and gradually tuning up the transmission through the LC nodule as the at least one color conversion film degrades and provides less fluorescence, to yield a constant output from the LCD (stage 590).

In method 500, the at least one color conversion film may be prepared by at least one corresponding sol-gel process (stages and method 600) and/or UV curing process (stage and method 700), which are presented in more detail below.

FIG. 17 is further a high-level flowchart illustrating a method 600 which may be part of method 500, according to some embodiments of the invention. The stages of method 600 may be carried out with respect to various aspects of precursors 72, formulations 74, films 130 and displays 100 described above, which may optionally be configured to implement method 600. Method 600 may comprise stages for producing, preparing and/or using precursors 72, formulations 74, films 130 and displays 100, such as any of the following stages, irrespective of their order.

Method 600 may comprise preparing a hybrid sol-gel precursor formulation from: an epoxy silica ormosil solution prepared from TEOS, at least one MTMOS or TMOS derivative, and GLYMO; a nanoparticles powder prepared from isocyanate-functionalized silica nanoparticles and ethylene glycol; and a metal(s) alkoxide matrix solution (stage 610), mixing the prepared hybrid sol-gel precursor with at least one RBF compound (stage 620); and spreading the mixture and drying the spread mixture to form a film (stage 630).

Method 600 may comprise comprising evaporating alcohols from the mixture prior to spreading 630 (stage 625). The inventors have found out that using ethylene glycol 108 in the preparation of nanoparticles powder 109 and evaporating 625 the alcohols prior to spreading improve film properties, and, for example, enable reducing the number of required green-fluorescent RBF layers 132 due to the increased viscosity of formulation 74. Possibly, the number of required green-fluorescent RBF layers 132 may be reduced to one by substantial or complete evaporation of the alcohols in formulation 74 prior to spreading 630.

Preparing 610 of the hybrid sol-gel precursor formulation may be carried out under acidic conditions (stage 612), mixing 620 may comprise adjusting types and amounts of the TMOS derivatives to tune emission wavelengths of the fluorophores (stage 615), spreading and drying 630 may be carried out respectively by bar coating and by at least one of convective heating, evaporating and infrared radiation (stage 640).

As explained above, the RBF compound may be a red-fluorescent RBF compound and the TMOS derivative(s) may comprise for example PhTMOS and/or a TMOS with fluorine substituents; and/or the RBF compound may be a green-fluorescent RBF compound and the TMOS derivative(s) may comprise PhTMOS and/or F₁TMOS with the PhTMOS:FiTMOS ratio being adjusted to tune emission properties of the green-fluorescent RBF compound. Other TMOS derivatives may comprise F₂TMOS (tridecafluoro-1,1,2,2-tetrahydrooctyl)trimethoxysilane, 1,2-bis(triethoxysilyl)ethane, trimethoxy(propyl)silane, octadecyltrimethoxysilane, fluorotriethoxysilane, and ammonium(propyl)trimethoxysilane.

Method 600 may comprise forming the film from at least one red fluorescent RBF compound and/or from at least one green fluorescent RBF compound (stage 650). The RBF compound(s) may be supramoleculary encapsulated and/or covalently embedded in one or more layers. As non-limiting examples, method 600 may comprise forming the film from at least one red fluorescent RBF compound to enhance a red illumination component in displays using a white light source (stage 680), such as a white-LED-based display. Alternatively or complementarily films may be formed to have both red and green fluorescent RBF compounds and be used for enhancing red and green illumination components in displays using a blue light source (blue LEDs).

Method 600 may comprise associating the film with any of diffuser(s), prism film(s) and polarizer film(s) in a display backlight unit (stage 660), e.g. attaching one or more films onto any of the elements in the display backlight unit or possibly replacing one or more of these elements by the formed film(s). For example, method 600 may comprise configuring the film to exhibit polarization properties (stage 670) and using the polarizing film to enhance or replace polarizer film(s) in the display backlight unit.

FIG. 17 is further a high-level flowchart illustrating a method 700 which may be part of method 500, according to some embodiments of the invention. The stages of method 700 may be carried out with respect to various aspects of formulations 74, films 130 and displays 100 described above, which may optionally be configured to implement method 700. Method 700 may comprise stages for producing, preparing and/or using formulations 74, films 130 and displays 100, such as any of the following stages, irrespective of their order.

Method 700 may comprise preparing a formulation from 65-70% monomers, 25-30% oligomers, 1-5% photoinitiator and at least one RBF compound (stage 710), in weight percentages of the total formulation, spreading the formulation to form a film (stage 730), and UV curing the formulation (stage 740). Method 700 may comprise any of: selecting the monomers from: dipropylene glycol diacrylate, ditrimethylolpropane tetraacrylate, dipentaerythritol hexaacrylate, ethoxylated pentaerythritol tetraacrylate, propoxylated (3) glyceryl acrylate and trimethylolpropane triacrylate; selecting the oligomers from: polyester acrylate, modified polyester resin diluted with dipropyleneglycol diacrylate and aliphatic urethane hexaacrylate; and selecting the photoinitiator from: alpha-hydroxy-cyclohexyl-phenyl-ketone and alpha-hydroxy ketone (possibly difunctional).

Method 700 may further comprise configuring the formulation and the film to yield a color conversion film and determining UV curing parameters to avoid damage to the color conversion elements, such as RBF compound(s) (stage 745). Method 700 may further comprise forming the color conversion film with at least one red fluorescent RBF compound and with at least one green fluorescent RBF compound (stage 750).

In some embodiments, method 700 may comprise configuring the color conversion film to exhibit polarization properties (stage 770), e.g., by directing self-assembly of molecules of the RBF compound(s) into at least partial alignment. Method 700 may further comprise associating the color conversion film with any of: a diffuser, a prism film and a polarizer film in a display backlight unit (stage 760).

In some embodiments, method 700 may comprise forming the color conversion film with at least one red fluorescent RBF compound to enhance a red illumination component in a white-LED-based display (stage 780) by shifting some of the yellow region in the emission spectrum of the white light source into the red region, namely into the R transmission region of the R color filter, to reduce illumination losses in the LCD panel while maintaining the balance between B and R+G regions in the RGB illumination (stage 782).

FIG. 17 is further a high-level flowchart further illustrating a method 800 of preparing BLUs with color conversion film(s) and/or elements(s), according to some embodiments of the invention. The method stages may be carried out with respect to LCDs 100 and BLUs 300 described above, which may optionally be configured to implement method 800. Method 800 may comprise stages for producing, preparing and/or using LCDs 100 and BLUs 300, such as any of the following stages, irrespective of their order.

Method 800 may comprise enhancing color conversion in a BLU, (stage 805) by incorporating at least one color conversion element, comprising at least one fluorescent dye configured to convert blue radiation to green and/or red radiation, within at least one partly reflective structure (stage 810) which is configured to redirect at least a part of radiation delivered from at least one illumination source of the BLU to a LCD—to pass multiple times through the at least one color conversion element (stage 820)—to set a white point of the delivered radiation according to specified requirements (stage 840). Method 800 may further comprise redirecting delivered radiation to pass multiple times through the at least one color conversion element (stage 825) as well as converting blue radiation to green and/or red radiation by fluorescent dyes (stage 830) to provide, with respect to the multiple passes of radiation therethrough, the required white point parameters as well as other illumination parameters such as intensity and spatial pattern, as disclosed above.

In certain embodiments, method 800 may further comprise delivering only some of the radiation through the at least one color conversion unit to the LCD panel, and delivering a part of the radiation directly to the LCD panel (stage 850), either globally, by redirecting part of the radiation directly toward the LCD panel, or locally, by designing illumination units to deliver only part of the radiation to the color conversion elements. Method 800 may further comprise configuring the part of the radiation directly to the LCD panel to increase a lifetime of the LCD, with respect to color conversion enhancement achieved by using the at least one partly reflective structure (stage 855).

FIG. 17 is further a high-level flowchart further illustrating a method 900 of modifying the BLU design to improve LCD performance, according to some embodiments of the invention. The method stages may be carried out with respect to LCD 100 and/or collimated backlight unit 300 described above, which may optionally be configured to implement method 900. Method 900 may comprise stages for producing, preparing and/or using LCD 100 and/or collimated backlight unit 300, such as any of the following stages, irrespective of their order.

Method 900 comprises providing collimated illumination to a LCD panel of a LCD (stage 910) by introducing illumination into at least one internally reflective cavity with a plurality of pinpoint openings (stage 920), and collimating illumination exiting the pinpoint openings by a corresponding of optical elements (stage 940).

In certain embodiments, method 900 may comprise configuring a plurality of internally reflective cavities, each with a corresponding one of the pinpoint openings at a focus point of a corresponding one of the optical elements (stage 930).

In certain embodiments, method 900 may comprise configuring one internally reflective cavity to receive illumination laterally and have the plurality of pinpoint openings at corresponding focus points of the optical elements (stage 935). Method 900 may further comprise perforating the cavity tops to provide multiple pinholes per cavity, to regulate the distribution of emitted radiation, and designing the optical elements accordingly (stage 937), e.g., to have multiple sub elements per each cavity, designed with respect to the multiple pinholes in each cavity. Possibly, method 900 may further comprise encapsulating the optical elements and/or sub elements within flat transparent material, to provide a flat optical element (stage 939).

In certain embodiments, method 900 may comprise designing one or more lenses (per illumination source) to collimate illumination from illumination sources (stage 950) and possibly molding the illumination sources in the designed lenses to collimate illumination therefrom (stage 955).

Method 900 may further comprise positioning lenslets of a lenslets array at a plane parallel to a plane defined by the pinpoint openings, with the pinpoint openings at the focus points of corresponding lenslets (stage 960). Method 900 may further comprise designing the optical elements and/or sub elements thereof to be encapsulated within flat transparent material, to provide a flat optical element (stage 965).

Method 900 may further comprise integrating the color conversion layer and the color filter layer (stage 970).

In certain embodiments, method 900 may comprise configuring the LCD to have the color conversion layer and the color filter layer above the LC module (stage 980).

In certain embodiments, method 900 may comprise setting a top optical-elements array on the LCD panel, configured to increase the brightness and radiance of the LCD (stage 990). In certain embodiments, method 900 may comprise designing the top optical-elements array to be encapsulated within flat transparent material, to provide a flat optical element (stage 992). In certain embodiments, method 900 may comprise using diffuser elements in any of R, G, B regions of the color conversion layer (stage 995).

In the above description, an embodiment is an example or implementation of the invention. The various appearances of “one embodiment”, “an embodiment”, “certain embodiments” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention may also be implemented in a single embodiment. Certain embodiments of the invention may include features from different embodiments disclosed above, and certain embodiments may incorporate elements from other embodiments disclosed above. The disclosure of elements of the invention in the context of a specific embodiment is not to be taken as limiting their use in the specific embodiment alone. Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described. Meanings of technical and scientific terms used herein are to be commonly understood as by one of ordinary skill in the art to which the invention belongs, unless otherwise defined. While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

What is claims is:

1. A LCD (liquid crystal display) comprising:
a backlight unit, and
a LCD panel receiving illumination radiation from the backlight unit, the LCD panel comprising:
a liquid crystal (LC) module,
RGB (red, green, blue) color filters, and
at least one color conversion film comprising:
at least one red-fluorescent rhodamine-based fluorescent (RBF) compound selected to have at least one R (red) emission peak,
at least one green RBF compound selected to have at least one G (green) emission peak, and
at least one assistant dye configured to modify a spectrum of received radiation.

2. The LCD of claim 1, wherein the at least one color conversion film is embedded in a fluorescence-intensifying section which comprises at least one supportive structure configured to redirect radiation to the at least one color conversion film.

3. The LCD of claim 2, wherein the fluorescence-intensifying section comprises at least one partly reflective layer positioned to receive radiation from, and reflect radiation to, the at least one color conversion film.

4. The LCD of claim 1, wherein the at least one color conversion film further comprises a crosstalk-reducing layer comprising a structural framework which is patterned according to a pixel structure of the RGB color filters.

5. The LCD of claim 1, wherein the at least one color conversion film is integrated with the RGB color filters and is patterned to yield a spatial correspondence between film regions with R and G emission peaks and respective R and G color filters.

6. The LCD of claim 5, wherein an integrated and patterned layer of the at least one color conversion film and the RGB color filters further comprises a crosstalk-reducing layer comprising a structural framework configured to reduce cross-talk between patterned pixels of the integrated layer.

7. The LCD of claim 1, wherein the red-fluorescent RBF compound is defined by Formula 1: 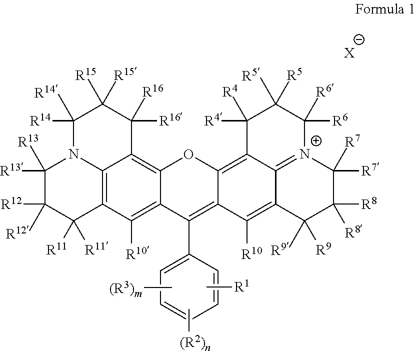
wherein:
R¹ is COOR, NO₂, COR, COSR, CO(N-heterocycle), CON(R)₂, or CN;
R² each is independently selected from H, halide, N(R)₂, COR, CN, CON(R)₂, CO(N-heterocycle), NCO, NCS, OR, SR, SO₃H, SO₃M and COOR;
R³ each is independently selected from H, halide, N(R)₂, COR, CN, CON(R)₂, CO(N-heterocycle), NCO, NCS, OR, SR, SO₃H, SO₃M and COOR;
R⁴-R¹⁶ and R^{4′}-R^{16′} are each independently selected from H, CF3, alkyl, haloalkyl, cycloalkyl, heterocycloalkyl, alkenyl, alkynyl, aryl, benzyl, halide, NO₂, OR, N(R)₂, COR, CN, CON(R)₂, CO(N-Heterocycle) and COOR;
R is H, alkyl, cycloalkyl, heterocycloalkyl, alkenyl, alkynyl, aryl, benzyl, —(CH₂CH₂O)ᵣCH₂CH₂OH, —(CH₂)ₚOC(O)NH(CH₂)_{q}Si(Oalkyl)₃, —(CH₂)ₚOC(O)CH═CH₂ or —(CH₂)ₚSi(Oalkyl)₃;
n and m are each independently an integer between 1-4;
p and q are each independently an integer between 1-6;
r is an integer between 0-10;
M is a monovalent cation; and
X⁻ is an anion; and
wherein the green-fluorescent RBF compound is defined by Formula 2: 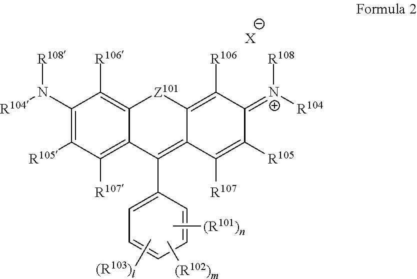
wherein:
R¹⁰¹ each is independently H, Q¹⁰¹, OQ¹⁰¹, C(O)Q¹⁰¹, NQ¹⁰¹Q¹⁰², NO₂, CN, SQ¹⁰¹, —NQ¹⁰¹Q¹⁰²CONQ¹⁰³Q¹⁰⁴, NCO, NCS, —OC(O)OQ¹ or halide;
R¹⁰² each is independently H, Q¹⁰¹, OQ¹⁰¹, C(O)Q¹⁰¹, NQ¹⁰¹Q¹⁰², NO₂, CN, SQ¹⁰¹, —NQ¹⁰¹Q¹⁰²CONQ¹⁰³Q¹⁰⁴, NCO, NCS, —OC(O)OQ¹⁰¹ or halide;
R¹⁰³ each is independently H, Q¹⁰¹, OQ¹⁰¹, C(O)Q¹⁰¹, NQ¹⁰¹Q¹⁰², NO₂, CN, SQ¹⁰¹, —NQ¹⁰¹Q¹⁰²CONQ¹⁰³Q¹⁰⁴, NCO, NCS, —OC(O)OQ¹⁰¹ or halide;
R¹⁰⁴, R^{104′}, R¹⁰⁸ and R^{108′} are each independently selected from H, alkyl, haloalkyl, heterocycloalkyl, cycloalkyl, aryl and benzyl;
R¹⁰⁵ and R^{105′} are each independently selected from H, Z′, OQ¹⁰¹, C(O)Q¹⁰¹, COOQ¹⁰¹, CON(Q¹⁰¹)₂, NQ¹⁰¹Q¹⁰², NO₂, CN, SO₃⁻, SO₃M, SO₃H, SQ¹⁰¹, —NQ¹⁰¹Q¹⁰²CONQ¹⁰³Q¹⁰⁴, NCO, NCS, alkenyl, alkynyl, epoxide, alkylated epoxide, alkylated azide, azide and halide;
R¹⁰⁶, R^{106′}, R¹⁰⁷ and R^{107′} are each independently selected from H, Q¹⁰¹, OQ¹⁰¹, C(O)Q¹⁰¹, COOQ¹⁰¹, CON(Q¹⁰¹)₂, NQ¹⁰¹Q¹⁰², NO₂, CN, SO₃⁻, SO₃M, SO₃H, SQ¹⁰¹, —NQ¹⁰¹Q¹⁰²CONQ¹⁰³Q¹⁰⁴, NCO, NCS, alkenyl, alkynyl, epoxide, alkylated epoxide, alkylated azide, azide and halide;
R¹⁰⁴ and R¹⁰⁵, R^{104′} and R^{105′}, R¹⁰⁴ and R¹⁰⁸ or R^{104′} and R^{108′} may form together an N-heterocyclic ring wherein said ring is optionally substituted;
Q¹⁰¹ and Q¹⁰² are each independently selected from H, alkyl, haloalkyl, heterocycloalkyl, cycloalkyl, aryl, benzyl, —(CH₂)ₚOC(O)NH(CH₂)_{q}Si(Oalkyl)₃, —(CH₂)ₚOC(O)CH═CH₂, —(CH₂)ₚOC(O)C(CH₃)═CH₂, —(CH₂)ₚSi(Oalkyl)₃, —(CH₂)ₚOC(O)NH(CH₂)_{q}Si(halide)₃, —(CH₂)ₚSi(halide)₃, —OC(O)N(H)Q¹⁰⁴, —OC(S)N(H)Q¹⁰⁴, —N(H)C(O)N(Q¹⁰³)₂ and —N(H)C(S)N(Q¹⁰³)₂;
Z¹⁰¹ is O or C(CH₃)₂;
Z′ is selected from alkyl, haloalkyl, heterocycloalkyl, cycloalkyl, aryl, benzyl, —(CH₂)ₚOC(O)NH(CH₂)_{q}Si(Oalkyl)₃, —(CH₂)ₚOC(O)CH═CH₂, —(CH₂)ₚOC(O)C(CH₃)═CH₂, —(CH₂)ₚSi(Oalkyl)₃, —(CH₂)ₚOC(O)NH(CH₂)_{q}Si(halide)₃, —(CH₂)ₚSi(halide)₃, —OC(O)N(H)Q¹⁰⁴, —OC(S)N(H)Q¹⁰⁴, —N(H)C(O)N(Q¹⁰³)₂ and —N(H)C(S)N(Q¹⁰³)₂;
Q¹⁰³ and Q¹⁰⁴ are each independently selected from H, alkyl, haloalkyl, heterocycloalkyl, cycloalkyl, aryl and benzyl;
M is a monovalent cation;
n, m and 1 are independently an integer between 1-5;
p and q are independently an integer between 1-6; and
X⁻ is an anion.

8. The LCD of claim 1, wherein the at least one assistant dye is defined by Formula (A1): 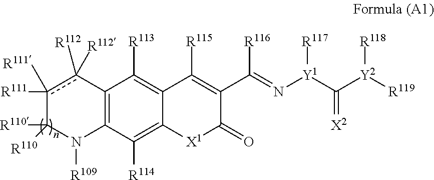
wherein
R¹⁰⁹ is H, alkyl, haloalkyl, heterocycloalkyl, cycloalkyl, aryl or benzyl;
R¹¹⁰ and R^{110′} are each independently H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹¹, R^{111′}, R¹¹² and R^{112′} are each independently absent, H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹³ is H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹⁴ is H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹⁵ is H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹⁶ is H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹⁷ is H, alkyl, haloalkyl, heterocycloalkyl, cycloalkyl, aryl or benzyl;
R¹¹⁸ and R¹¹⁹ are each independently H, alkyl, haloalkyl, heterocycloalkyl, cycloalkyl, aryl or benzyl;
X¹ is NR^{a}, S or O;
X² is NR^{a}, S or O
Y¹ is N or CR^{a};
Y² is N or CR^{a};
R^{a} is H, alkyl, haloalkyl, cycloalkyl, heterocycloalkyl, aryl or benzyl;
n is an integer between 0 and 3; and
wherein if there is a double bond between the carbons which are substituted by R¹¹¹, R^{111′}, R¹¹² and R^{112′}—then one of R¹¹¹ and R^{111′} is absent and one of R¹¹² and R^{112′} is absent.

9. The LCD of claim 1, wherein the at least one assistant dye is defined by Formula (A2): 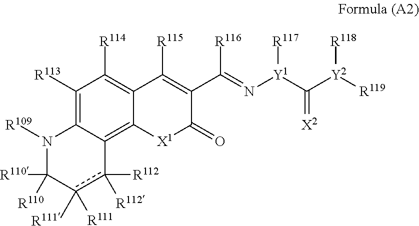
wherein
R¹⁰⁹ is H, alkyl, haloalkyl, heterocycloalkyl, cycloalkyl, aryl or benzyl;
R¹¹⁰ and R^{110′} are each independently H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹¹, R^{111′}, R¹¹² and R^{112′} are each independently absent, H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹³ is H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹⁴ is H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹⁵ is H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹⁶ is H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹⁷ is H, alkyl, haloalkyl, heterocycloalkyl, cycloalkyl, aryl or benzyl;
R¹¹⁸ and R¹¹⁹ are each independently H, alkyl, haloalkyl, heterocycloalkyl, cycloalkyl, aryl or benzyl;
X¹ is NR^{a}, S or O;
X² is NR^{a}, S or O
Y¹ is N or CR^{a};
Y² is N or CR^{a};
R^{a} is H, alkyl, haloalkyl, cycloalkyl, heterocycloalkyl, aryl or benzyl; and
wherein if there is a double bond between the carbons which are substituted by R¹¹¹, R^{111′}, R¹¹² and R^{112′}—then one of R¹¹¹ and R^{111′} is absent and one of R¹¹² and R^{112′} is absent.

10. The LCD of claim 1, wherein the at least one assistant dye is defined by Formula (A3): 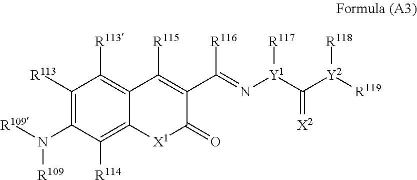
wherein
R¹⁰⁹ is H, alkyl, haloalkyl, heterocycloalkyl, cycloalkyl, aryl or benzyl;
R¹¹³ is H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹⁴ is H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹⁵ is H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹⁶ is H, alkyl, haloalkyl, cycloalkyl, heterocycloalkyl, aryl, benzyl, halide, NO₂, OR^{a}, N(R^{a})₂, COR^{a}, CN, CON(R^{a})₂, CO(N-heterocycle) or COOR^{a};
R¹¹⁷ is H, alkyl, haloalkyl, heterocycloalkyl, cycloalkyl, aryl or benzyl;
R¹¹⁸ and R¹¹⁹ are each independently H, alkyl, haloalkyl, heterocycloalkyl, cycloalkyl, aryl or benzyl;
X¹ is NR^{a}, S or O;
X² is NR^{a}, S or O
Y¹ is N or CR^{a};
Y² is N or CR^{a}; and
R^{a} is H, alkyl, haloalkyl, cycloalkyl, heterocycloalkyl, aryl or benzyl.

11. The LCD of claim 1, wherein the at least one assistant dye comprises at least one of compounds A1-, A1-2, A1-3, A2-1, A2-2 and A3-1, represented by the following structures: 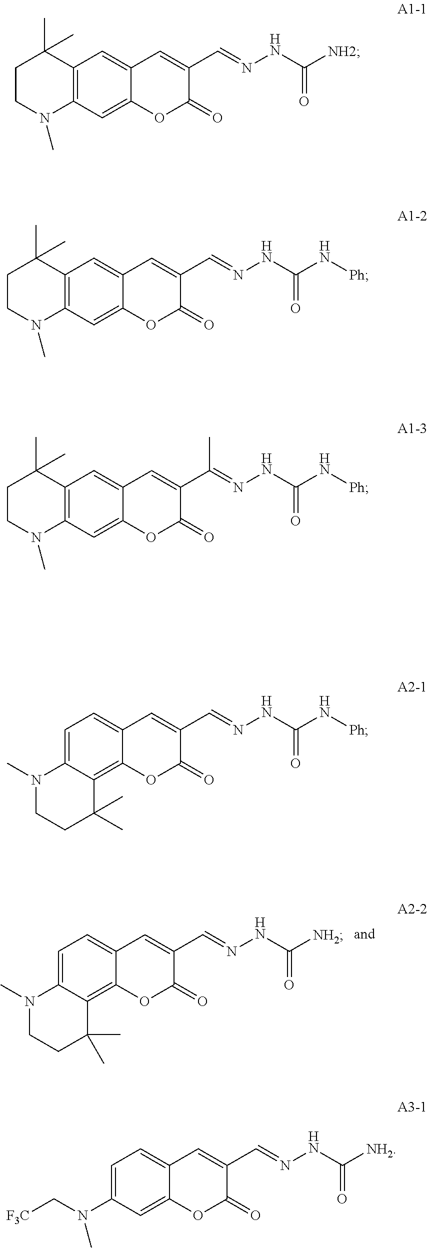 [SMILES: CN1CCC(C)(C)c2cc3cc(/C=N/NC(N)=O)c(=O)oc3cc21 ; CN1CCC(C)(C)c2cc3cc(/C=N/NC(=O)Nc4ccccc4)c(=O)oc3cc21 ; C/C(=N\NC(=O)Nc1ccccc1)c1cc2cc3c(cc2oc1=O)N(C)CCC3(C)C ; CN1CCC(C)(C)c2c1ccc1cc(/C=N/NC(=O)Nc3ccccc3)c(=O)oc21 ; CN1CCC(C)(C)c2c1ccc1cc(/C=N/NC(N)=O)c(=O)oc21 ; CN(CC(F)(F)F)c1ccc2cc(/C=N/NC(N)=O)c(=O)oc2c1]
